(19) United States
(12) Patent Application Publication
(10) Pub. No.: US 2022/0346436 A1
(43) Pub. Date: Nov. 3, 2022

(54) ORALLY DISSOLVING FILMS

(71) Applicant: NICOVENTURES TRADING LIMITED, London (GB)

(72) Inventors: Michael Andrew Zawadzki, Clemmons, NC (US); Steven Lee Alderman, Lewisville, NC (US); Thomas H. Poole, Winston-Salem, NC (US); Michael Joyce, Athens, GA (US); Wesley Steven Jones, Lexington, NC (US); Matthew Lampe, Winston-Salem, NC (US)

(21) Appl. No.: 17/726,121

(22) Filed: Apr. 21, 2022

Related U.S. Application Data
(60) Provisional application No. 63/178,322, filed on Apr. 22, 2021.

(51) Int. Cl. A24B 15/30; A61K 47/12; A61K 9/00; A61K 47/38; A61K 31/465

(52) U.S. Cl. CPC A24B 15/30; A61K 47/12; A61K 9/0053; A61K 47/38; A61K 31/465; A61K 9/009

(57) ABSTRACT

An oral product in the form of an edible film is provided herein. The edible film can include a binder in an amount of at least about 30 percent by weight; a plasticizer in an amount of at least about 5 percent by weight; and an active agent, wherein the edible film is orally dissolvable. The edible film can also be used as a component of another oral product, e.g., a fleece-based pouched product. The edible film can form a coating on a portion of an interior or exterior surface of a fleece-based pouched product.

### CROSS-REFERENCE TO RELATED APPLICATIONS

The present application claims priority to and the benefit of U.S. Provisional Patent Application No. 63/178,322, filed Apr. 22, 2021, the disclosure of which is incorporated herein by reference in its entirety.

### FIELD OF THE DISCLOSURE

The present disclosure relates to oral products intended for human use. The products are configured for oral use and deliver substances such as flavors and/or active ingredients during use. Such products may include tobacco or a product derived from tobacco, or may be tobacco-free alternatives.

### BACKGROUND

Tobacco may be enjoyed in a so-called “smokeless” form. Particularly popular smokeless tobacco products are employed by inserting some form of processed tobacco or tobacco-containing formulation into the mouth of the user. Conventional formats for such smokeless tobacco products include moist snuff, snus, and chewing tobacco, which are typically formed almost entirely of particulate, granular, or shredded tobacco, and which are either portioned by the user or presented to the user in individual portions, such as in single-use pouches or sachets. Other traditional forms of smokeless products include compressed or agglomerated forms, such as plugs, tablets, or pellets. Alternative product formats, such as tobacco-containing gums and mixtures of tobacco with other plant materials, are also known. See for example, the types of smokeless tobacco formulations, ingredients, and processing methodologies set forth in U.S. Pat. Nos. 1,376,586 to Schwartz; 4,513,756 to Pittman et al.; 4,528,993 to Sensabaugh, Jr. et al.; 4,624,269 to Story et al.; 4,991,599 to Tibbetts; 4,987,907 to Townsend; 5,092,352 to Sprinkle, III et al.; 5,387,416 to White et al.; 6,668,839 to Williams; 6,834,654 to Williams; 6,953,040 to Atchley et al.; 7,032,601 to Atchley et al.; and 7,694,686 to Atchley et al.; US Pat. Pub. Nos. 2004/0020503 to Williams; 2005/0115580 to Quinter et al.; 2006/0191548 to Strickland et al.; 2007/0062549 to Holton, Jr. et al.; 2007/0186941 to Holton, Jr. et al.; 2007/0186942 to Strickland et al.; 2008/0029110 to Dube et al.; 2008/0029116 to Robinson et al.; 2008/0173317 to Robinson et al.; 2008/0209586 to Neilsen et al.; 2009/0065013 to Essen et al.; and 2010/0282267 to Atchley, as well as WO2004/095959 to Arnarp et al., each of which is incorporated herein by reference.

Smokeless tobacco product configurations that combine tobacco material with various binders and fillers have been proposed more recently, with example product formats including lozenges, pastilles, gels, extruded forms, and the like. See, for example, the types of products described in US Patent App. Pub. Nos. 2008/0196730 to Engstrom et al.; 2008/0305216 to Crawford et al.; 2009/0293889 to Kumar et al.; 2010/0291245 to Gao et al; 2011/0139164 to Mua et al.; 2012/0037175 to Cantrell et al.; 2012/0055494 to Hunt et al.; 2012/0138073 to Cantrell et al.; 2012/0138074 to Cantrell et al.; 2013/0074855 to Holton, Jr.; 2013/0074856 to Holton, Jr.; 2013/0152953 to Mua et al.; 2013/0274296 to Jackson et al.; 2015/0068545 to Moldoveanu et al.; 2015/0101627 to Marshall et al.; and 2015/0230515 to Lampe et al., each of which is incorporated herein by reference. Oral products in similar formats and which are free of tobacco have also been proposed.

It would be desirable to provide products configured for oral use which may deliver active ingredients to the consumer in an enjoyable form.

### BRIEF SUMMARY

These and other features, aspects, and advantages of the disclosure will be apparent from a reading of the following detailed description together with the accompanying drawings, which are briefly described below. The invention includes any combination of two, three, four, or more of the above-noted embodiments as well as combinations of any two, three, four, or more features or elements set forth in this disclosure, regardless of whether such features or elements are expressly combined in a specific embodiment description herein. This disclosure is intended to be read holistically such that any separable features or elements of the disclosed invention, in any of its various aspects and embodiments, should be viewed as intended to be combinable unless the context clearly dictates otherwise.

The disclosure provides, in one aspect, an oral product comprising an edible film, wherein the edible film comprises: a binder in an amount of at least about 30 percent by weight; a plasticizer in an amount of at least about 5 percent by weight; and an active agent; wherein the edible film is orally dissolvable.

In one embodiment, the binder is selected from the group consisting of film-forming polysaccharides, starch, modified starch, methyl cellulose, modified cellulose, pullulan, pectin, carrageenan, alginate, gums (e.g., locust bean gum), agar, and combinations thereof. For example, in certain embodiments, the binder comprises hydroxypropylmethylcellulose, carboxymethylcellulose, modified corn starch, or a combination thereof. The binder can be present, e.g., in an amount of at least about 35 percent by weight, at least about 50%, or at least about 60%, based on the weight of the edible film.

In certain embodiments, the plasticizer is selected from the group consisting of glycerin, propylene glycol, and combinations thereof. The plasticizer can be present, e.g., in an amount of at least about 10 percent by weight.

In some embodiments, the edible film further comprises a filler. Examples of suitable fillers include, but are not limited to, fillers selected from the group consisting of carbohydrates, cellulose powder, fiber, starch, maltodextrin, polyglycitols, polysaccharides, minerals, and combinations thereof. In some embodiments, the edible film further comprises a processing aid. Examples of processing aids include, e.g., those selected from the group consisting of surfactants and emulsifiers. Certain specific processing aids used in some embodiments include, e.g., lecithin, polysorbates (e.g., polysorbate 80), polyoxyl stearate (e.g., polyoxyl 40 stearate), glycerol monostearate, and combinations thereof. In some embodiments, the edible film further comprises a flavorant, a colorant, a sweetener, or any combination thereof. In some embodiments, the edible film further comprises an anti-stick coating.

In some embodiments, the active ingredient is selected from the group consisting of a nicotine component, botanicals, stimulants, nutraceuticals, amino acids, vitamins, cannabinoids, cannabimimetics, terpenes, and combinations thereof. For example, in various embodiments, the active ingredient is a nicotine component selected from nicotine benzoate and nicotine polacrilex. The oral product can, in some embodiments, comprise a basic amine (e.g., nicotine) and an organic acid, an alkali metal salt of an organic acid, or a combination thereof, wherein the organic acid has a logP value of from about 1.0 to about 12.0 and at least a portion of the basic amine is associated with at least a portion of the organic acid or the alkali metal salt thereof, the association in the form of a basic amine-organic acid salt, an ion pair between the basic amine and a conjugate base of the organic acid, or both. In various embodiments, the organic acid can have a logP value of from about 1.4 to about 4.5 or about 2.5 to about 3.5. In some embodiments, the organic acid can have a logP value of from about 4.5 to about 8.0, and wherein the composition further comprises a solubility enhancer (e.g., glycerol and/or propylene glycol). In some embodiments, the oral product comprises from about 0.05, about 0.1, about 1, about 1.5, about 2, or about 5 to about 10, about 15, or about 20 molar equivalents of the organic acid, the alkali metal salt thereof, or the combination thereof relative to the basic amine, calculated as the amine free base (e.g., from about 2 to about 10 molar equivalents). In some embodiments, the organic acid is an alkyl carboxylic acid, an aryl carboxylic acid, an alkyl sulfonic acid, an aryl sulfonic acid, a menthyl or tocopherol monoester of a dicarboxylic acid, or a combination of any thereof. For example, the organic acid can be octanoic acid, decanoic acid, benzoic acid, heptanesulfonic acid, tocopherol succinate, monomenthyl succinate, monomenthyl fumarate, monomenthyl glutarate, or a combination of any thereof. In some embodiments the organic acid is benzoic acid. In some embodiments, the alkali metal is sodium or potassium. In some embodiments, the oral product comprises the organic acid and a sodium salt of the organic acid (e.g., benzoic acid and sodium benzoate). In some such embodiments, a ratio of the organic acid to the sodium salt of the organic acid is from about 0.1 to about 10.

In some embodiments, the active ingredient comprises a stimulant. Examples of suitable stimulants include, but are not limited to, stimulants selected from the group consisting of caffeine, theanine, or a combination thereof. In some embodiments, the active ingredient comprises one or more of theanine, gamma-aminobutyric acid, caffeine, and taurine. In certain embodiments, the edible film is substantially free of a tobacco material. In certain embodiments, the oral product is substantially free of a tobacco material. In some embodiments, the oral product comprises a tobacco material, e.g., specifically a whitened tobacco material.

The oral product can, in some embodiments, consist essentially of the edible film. For example, in certain embodiments, the edible film is in the form of a strip sized for a consumer's oral cavity. In some embodiments, the oral product and/or the edible film can have a basis weight of about 100 to about 400 mg for a dimension of about 33 mm×11 mm (understood to be applicable to films of varying dimensions by scaling accordingly). The moisture content of the edible film can vary; in some embodiments, the edible film has a moisture content of about 5 to about 10 weight percent.

In some embodiments, the oral product is in the form of a pouched product comprising the edible film, the oral product comprising an outer pouch defining a cavity, wherein the outer pouch comprises a fleece material. In some such embodiments, the edible film is a coating on at least a portion of an interior or exterior surface of the fleece material. In some such embodiments, the edible film is a coating on substantially all of the interior or exterior surface of the fleece material. In some such embodiments, the edible film is a coating on specific areas of the interior or exterior surface of the fleece material. In some such embodiments, the edible film is within the cavity of the pouch.

The film associated with the pouched product can vary. In some embodiments, the edible film comprises nicotine. In some embodiments, the edible film comprises a stimulant. Examples of suitable stimulants include, but are not limited to, stimulants selected from the group consisting of caffeine, theanine, or a combination thereof. In some embodiments, the active ingredient comprises one or more of theanine, gamma-aminobutyric acid, caffeine, and taurine. In certain embodiments, the edible film is substantially free of a tobacco material. In certain embodiments, the oral product is substantially free of a tobacco material. In some embodiments, the film comprises a tobacco material, e.g., specifically a whitened tobacco material.

In some embodiments, the pouched product further comprises an oral composition in the cavity of the pouch. The oral composition can comprise, for example, at least one of an active agent and a flavorant in an amount of at least about 0.5% by weight of the oral composition; and a filler in an amount of at least about 30% by weight of the oral composition. In some embodiments, the filler is selected from the group consisting of a sugar substitute, microcrystalline cellulose, or a combination thereof.

In another aspect is provided an oral product in the form of a pouched product, comprising: an outer pouch defining a cavity, and an oral composition situated within the cavity, wherein the outer pouch comprises an edible film, comprising: a binder in an amount of at least about 30 percent by weight and a plasticizer in an amount of at least about 5 percent by weight, wherein the edible film is orally dissolvable.

The disclosure includes, without limitation, the following embodiments:

### Embodiment 1

An oral product comprising an edible film, wherein the edible film comprises: a binder in an amount of at least about 30 percent by weight; a plasticizer in an amount of at least about 5 percent by weight; and an active agent; wherein the edible film is orally dissolvable.

### Embodiment 2

The oral product of Embodiment 1, wherein the binder is selected from the group consisting of film-forming polysaccharides, starch, modified starch, methyl cellulose, modified cellulose, pullulan, pectin, carrageenan, alginate, gums (e.g., locust bean gum), agar, and combinations thereof.

### Embodiment 3

The oral product of any of Embodiments 1-2, wherein the binder comprises hydroxypropylmethylcellulose, carboxymethylcellulose, modified corn starch, or a combination thereof.

### Embodiment 4

The oral product of any of Embodiments 1-3, wherein the binder is present in an amount of at least about 35 percent by weight.

### Embodiment 5

The oral product of any of Embodiments 1-4, wherein the plasticizer is selected from the group consisting of glycerin, propylene glycol, and combinations thereof.

### Embodiment 6

The oral product of any of Embodiments 1-5, wherein the plasticizer is present in an amount of at least about 10 percent by weight.

### Embodiment 7

The oral product of any of Embodiments 1-6, wherein the edible film further comprises a filler.

### Embodiment 8

The oral product of Embodiment 7, wherein the filler is selected from the group consisting of carbohydrates, cellulose powder, fiber, starch, maltodextrin, polyglycitols, polysaccharides, minerals, and combinations thereof.

### Embodiment 9

The oral product of any of Embodiments 1-8, wherein the edible film further comprises a processing aid.

### Embodiment 10

The oral product of Embodiment 9, wherein the processing aid is selected from the group consisting of surfactants and emulsifiers.

### Embodiment 11

The oral product of Embodiment 9, wherein the processing aid is selected from lecithin, polysorbates (e.g., polysorbate 80), polyoxyl stearate (e.g., polyoxyl 40 stearate), glycerol monostearate, and combinations thereof.

### Embodiment 12

The oral product of any of Embodiments 1-11, wherein the edible film further comprises a flavorant, a colorant, a sweetener, or any combination thereof.

### Embodiment 13

The oral product of any of Embodiments 1-12, wherein the edible film further comprises an anti-stick coating.

### Embodiment 14

The oral product of any of Embodiments 1-13, wherein the active ingredient is selected from the group consisting of a nicotine component, botanicals, stimulants, nutraceuticals, amino acids, vitamins, cannabinoids, cannabimimetics, terpenes, and combinations thereof.

### Embodiment 15

The oral product of Embodiment 14, wherein the active ingredient is a nicotine component selected from nicotine benzoate and nicotine polacrilex.

### Embodiment 16

The oral product of any of Embodiments 1-14, comprising a basic amine (e.g., nicotine) and an organic acid, an alkali metal salt of an organic acid, or a combination thereof, wherein the organic acid has a logP value of from about 1.0 to about 12.0 and at least a portion of the basic amine is associated with at least a portion of the organic acid or the alkali metal salt thereof, the association in the form of a basic amine-organic acid salt, an ion pair between the basic amine and a conjugate base of the organic acid, or both.

### Embodiment 17

The oral product of Embodiment 16, wherein the organic acid has a logP value of from about 1.4 to about 4.5, e.g., about 2.5 to about 3.5.

### Embodiment 18

The oral product of Embodiment 16, wherein the organic acid has a logP value of from about 4.5 to about 8.0 and wherein the composition further comprises a solubility enhancer (e.g., glycerol or propylene glycol).

### Embodiment 19

The oral product of any of Embodiments 16-18, comprising from about 0.05, about 0.1, about 1, about 1.5, about 2, or about 5 to about 10, about 15, or about 20 (e.g., about 2 to about 10) molar equivalents of the organic acid, the alkali metal salt thereof, or the combination thereof, relative to the basic amine, calculated as the amine free base.

### Embodiment 20

The oral product of any of Embodiments 16-19, wherein the organic acid is an alkyl carboxylic acid, an aryl carboxylic acid, an alkyl sulfonic acid, an aryl sulfonic acid, a menthyl or tocopherol monoester of a dicarboxylic acid, or any combination thereof.

### Embodiment 21

The oral product of Embodiment 20, wherein the organic acid is octanoic acid, decanoic acid, benzoic acid, heptanesulfonic acid, tocopherol succinate, monomenthyl succinate, monomenthyl fumarate, monomenthyl glutarate, or a combination of any thereof.

### Embodiment 22

The oral product of any of Embodiments 16-21, wherein the alkali metal is sodium or potassium.

### Embodiment 23

The oral product of any of Embodiments 16-22, comprising the organic acid and a sodium salt of the organic acid (e.g., benzoic acid and sodium benzoate).

### Embodiment 24

The oral product of Embodiment 23, wherein a ratio of the organic acid to the sodium salt of the organic acid is from about 0.1 to about 10.

### Embodiment 25

The oral product of any of Embodiments 16-24, wherein the pH of the oral product is from about 4.0 to about 9.0.

### Embodiment 26

The oral product of Embodiment 14, wherein the active ingredient comprises a stimulant.

### Embodiment 27

The oral product of Embodiment 26, wherein the stimulant is selected from the group consisting of caffeine, theanine, or a combination thereof.

### Embodiment 28

The oral product of Embodiment 14, wherein the active ingredient comprises one or more of theanine, gamma-aminobutyric acid, caffeine, and taurine.

### Embodiment 29

The oral product of any of Embodiments 1-28, wherein the edible film is substantially free of a tobacco material.

### Embodiment 30

The oral product of any of Embodiments 1-29, wherein the oral product is substantially free of a tobacco material.

### Embodiment 31

The oral product of any of Embodiments 1-29, wherein the oral product comprises a whitened tobacco material.

### Embodiment 32

The oral product of any of Embodiments 1-31, consisting essentially of the edible film.

### Embodiment 33

The oral product of any of Embodiments 1-32, wherein the edible film has a basis weight of about 100 mg to about 400 mg for a dimension of about 33 mm×11 mm.

### Embodiment 34

The oral product of any of Embodiments 1-33, wherein the edible film is in the form of a strip sized for a consumer's oral cavity.

### Embodiment 35

The oral product of any of Embodiments 1-34, wherein the edible film has a moisture content of about 5 to about 10 weight percent.

### Embodiment 36

The oral product of any of Embodiments 1-31 or 33-35, in the form of a pouched product comprising the edible film, the oral product comprising an outer pouch defining a cavity, wherein the outer pouch comprises a fleece material.

### Embodiment 37

The oral product of Embodiment 36, wherein the edible film is a coating on at least a portion of an interior or exterior surface of the fleece material.

### Embodiment 38

The oral product of any of Embodiments 36-37, wherein the edible film is a coating on substantially all of the interior or exterior surface of the fleece material.

### Embodiment 39

The oral product any of Embodiments 36-38, wherein the edible film is a coating on specific areas of the interior or exterior surface of the fleece material.

### Embodiment 40

The oral product of any of Embodiments 36-39, wherein the edible film is within the cavity of the pouch.

### Embodiment 41

The oral product of any of Embodiments 36-40, wherein the edible film comprises nicotine.

### Embodiment 42

The oral product of any of Embodiments 36-41, further comprising an oral composition in the cavity of the pouch.

### Embodiment 43

The oral product of Embodiment 42, wherein the oral composition comprises: at least one of an active agent and a flavorant in an amount of at least about 0.5% by weight of the oral composition; and a filler in an amount of at least about 30% by weight of the oral composition.

### Embodiment 44

The oral product of Embodiment 43, wherein the filler is selected from the group consisting of a sugar substitute, microcrystalline cellulose, or a combination thereof.

### Embodiment 45

An oral product in the form of a pouched product, comprising: an outer pouch defining a cavity, and an oral composition situated within the cavity, wherein the outer pouch comprises an edible film, comprising: a binder in an amount of at least about 30 percent by weight and a plasticizer in an amount of at least about 5 percent by weight, wherein the edible film is orally dissolvable.

### Embodiment 46

The oral product of Embodiment 45, wherein the oral composition comprises at least about 30% by weight of a filler.

### Embodiment 47

The oral product of Embodiment 46, wherein the filler comprises microcrystalline cellulose or isomalt or a combination thereof.

### Embodiment 48

The oral product of any of Embodiments 45-47, wherein the oral composition comprises one or more active ingredients selected from the group consisting of a nicotine component, botanicals, stimulants, nutraceuticals, amino acids, vitamins, cannabinoids, cannabimimetics, terpenes, and combinations thereof.

### Embodiment 49

The oral product of Embodiment 48, wherein the active ingredient is a nicotine component selected from nicotine benzoate and nicotine polacrilex.

### Embodiment 50

The oral product of Embodiments 48, comprising a basic amine (e.g., nicotine) and an organic acid, an alkali metal salt of an organic acid, or a combination thereof, wherein the organic acid has a logP value of from about 1.0 to about 12.0 and at least a portion of the basic amine is associated with at least a portion of the organic acid or the alkali metal salt thereof, the association in the form of a basic amine-organic acid salt, an ion pair between the basic amine and a conjugate base of the organic acid, or both.

### Embodiment 51

The oral product of Embodiment 50, wherein the organic acid has a logP value of from about 1.4 to about 4.5, e.g., about 2.5 to about 3.5.

### Embodiment 52

The oral product of Embodiment 50, wherein the organic acid has a logP value of from about 4.5 to about 8.0 and wherein the composition further comprises a solubility enhancer (e.g., glycerol or propylene glycol).

### Embodiment 53

The oral product of any of Embodiments 50-52, comprising from about 0.05, about 0.1, about 1, about 1.5, about 2, or about 5 to about 10, about 15, or about 20 (e.g., about 2 to about 10) molar equivalents of the organic acid, the alkali metal salt thereof, or the combination thereof, relative to the basic amine, calculated as the amine free base.

### Embodiment 54

The oral product of any of Embodiments 50-53, wherein the organic acid is an alkyl carboxylic acid, an aryl carboxylic acid, an alkyl sulfonic acid, an aryl sulfonic acid, a menthyl or tocopherol monoester of a dicarboxylic acid, or any combination thereof.

### Embodiment 55

The oral product of Embodiment 54, wherein the organic acid is octanoic acid, decanoic acid, benzoic acid, heptanesulfonic acid, tocopherol succinate, monomenthyl succinate, monomenthyl fumarate, monomenthyl glutarate, or a combination of any thereof.

### Embodiment 56

The oral product of any of Embodiments 50-55, wherein the alkali metal is sodium or potassium.

### Embodiment 57

The oral product of any of Embodiments 50-56, comprising the organic acid and a sodium salt of the organic acid (e.g., benzoic acid and sodium benzoate).

### Embodiment 58

The oral product of Embodiment 57, wherein a ratio of the organic acid to the sodium salt of the organic acid is from about 0.1 to about 10.

### Embodiment 59

The oral product of any of Embodiments 50-58, wherein the pH of the oral product is from about 4.0 to about 9.0.

### Embodiment 60

The oral product of Embodiment 48, wherein the active ingredient comprises a stimulant.

### Embodiment 61

The oral product of Embodiment 60, wherein the stimulant is selected from the group consisting of caffeine, theanine, or a combination thereof.

### Embodiment 62

The oral product of Embodiment 61, wherein the caffeine is encapsulated caffeine.

### Embodiment 63

The oral product of Embodiment 48, wherein the active ingredient comprises one or more of theanine, gamma-aminobutyric acid, caffeine, taurine, vitamin C, lemon balm extract, and combinations thereof.

These and other features, aspects, and advantages of the disclosure will be apparent from a reading of the following detailed description together with the accompanying drawings, which are briefly described below. The invention includes any combination of two, three, four, or more of the above-noted embodiments as well as combinations of any two, three, four, or more features or elements set forth in this disclosure, regardless of whether such features or elements are expressly combined in a specific embodiment description herein. This disclosure is intended to be read holistically such that any separable features or elements of the disclosed invention, in any of its various aspects and embodiments, should be viewed as intended to be combinable unless the context clearly dictates otherwise.

### BRIEF DESCRIPTION OF THE DRAWINGS

Having thus described aspects of the disclosure in the foregoing general terms, reference will now be made to the accompanying drawings, which are not necessarily drawn to scale. The drawings are example only, and should not be construed as limiting the disclosure.

FIGS. 1A, 1B, 1C, 1D, 1E, 1F, and 1G are non-limiting examples of suitable shapes for oral products in the form of films according to certain embodiments of the present disclosure;

FIGS. 2A and 2B are examples of preparations of two film-based pouched products according to certain embodiments of the present disclosure;

FIG. 3 is a perspective view of a fleece-based pouched product according to an embodiment of the present disclosure;

FIGS. 4A, 4B, and 4C are cross-sectional views illustrating fleece-based pouched products comprising a film according to various embodiments of the present disclosure;

FIGS. 5A, 5B, 5C, 5D, 5E, 5F, 5G, 5H, 5I, and 5J are top (surface) views of various, non-limiting designs of fleece-based pouched products comprising film material positioned on the outside of the pouched products according to various embodiments of the present disclosure; and

FIGS. 6A, 6B, 6C, and 6D are schematic drawings of various fleece-based pouches with film material associated therewith according to various embodiments of the present disclosure.

### DETAILED DESCRIPTION

The present invention now will be described more fully hereinafter. This invention may, however, be embodied in many different forms and should not be construed as limited to the embodiments set forth herein; rather, these embodiments are provided so that this disclosure will be thorough and complete, and will fully convey the scope of the invention to those skilled in the art. As used in this specification and the claims, the singular forms “a,” “an,” and “the” include plural referents unless the context clearly dictates otherwise.

The disclosure generally provides products configured for oral use. The term “configured for oral use” as used herein means that the product is provided in a form such that during use, saliva in the mouth of the user causes one or more of the components of the mixture (e.g., flavoring agents and/or nicotine) to pass into the mouth of the user. In certain embodiments, the product is adapted to deliver one or more components to a user through mucous membranes in the user's mouth and, in some instances, said component is an active ingredient (including, but not limited to, for example, nicotine) that can be absorbed through the mucous membranes in the mouth when the product is used.

In particular, the disclosure provides products comprising an edible, orally dissolvable film. The term “film” is used herein to mean a formulation intended for oral administration that is orally dissolvable and suitable for human consumption. These edible films generally are in the form of thin strips/sheets/tapes/pieces designed to be placed within the oral cavity of a consumer and/or combined in various ways with other materials to form an oral product. The films provided herein typically provide for sublingual and/or buccal delivery of one or more active agents. The films can be provided alone or in combination with one or more additional (e.g., non-film) components to provide an oral product.

The components of the disclosed films can vary. Typically, each edible, orally dissolvable film comprises various edible materials, including a binder component and a plasticizer component and, in some embodiments, one or more active agents and/or one or more flavorants. Examples of the types of components that can be incorporated within the disclosed edible, orally dissolvable films are provided herein below.

### Binder Component

The films generally comprise a binder component comprising one or more binders, which typically provide, e.g., particular shape, strength, oral dissolution rate, and/or mouthfeel characteristics to the films. Binders as used in the edible, orally dissolvable films provided herein generally comprise one or more film-forming materials and often comprise two or more film-forming materials (i.e., the binder component is a mixture of ingredients). Film forming materials are known and can include, e.g., film-forming polysaccharides, starch, modified corn starch, modified celluloses, pullulan, pectin, carrageenan, alginate (including, e.g., cross-linked alginate), gums (e.g., locust bean gum), agar, and other film formers, e.g., natural film formers.

Certain specific examples of modified celluloses that can be employed as components of the binder include, but are not limited to, hydroxypropylmethyl cellulose (HPMC), methyl cellulose, and carboxymethylcellulose. In some embodiments, two or more HPMCs are employed. HPMCs can vary, e.g., by viscosity, particle properties, polymer molecular weight, and by average content of methoxy groups and average content of hydroxpropyl groups, as well as substitution pattern. HPMC binders that can be employed suitably in the disclosed products are not particularly limited. Various types of HPMC are available, e.g., from JRS Pharma (e.g., Vivapharm ® HPMC, e.g., grade E5), Dow (e.g., Methocel™ e.g., grade K99), Lotte Fine Chemical (e.g., AnyAddy® HPMC), and others, which are also encompassed by the present disclosure.

Modified corn starches can include, e.g., chemically modified starches (e.g., OSA starch) and acid-modified starch. Certain specific examples of modified corn starches that can be employed include, but are not limited to, corn starches that have been treated to improve the consistency thereof, e.g., corn starch that has been roasted, treated with acid, treated with an electrical starch, or treated with sodium hydroxide or potassium hydroxide. In particular, corn starches that form a film when dried are applicable as binders according to the present disclosure; a modified starch can be selected in some embodiments to give desired mechanical, tactile, and/or sensory properties (for example flexibility, low tack, neutral sensory characteristics). In other embodiments, these properties can be modified by the other components of the composition. One particular starch that can be employed according to the present disclosure is Pure Cote® B792, available from Grain Processing Corporation, which is an acid-hydrolyzed starch that was designed for producing clear, flexible films without heating to hydrate the starch.

In certain embodiments, the binder includes a gum, for example, a natural gum. As used herein, a natural gum refers to polysaccharide materials of natural origin that have binding properties, and which are also useful as a thickening or gelling agents. Representative natural gums derived from plants, which are typically water soluble to some degree, include xanthan gum, guar gum, gum arabic, ghatti gum, gum tragacanth, karaya gum, locust bean gum, gellan gum, agar, and combinations thereof. Binders are generally present in an amount of at least about 20% by weight, at least about 30% by weight, or at least about 40% by weight, based on the total weight of the edible, orally dissolvable film. In some embodiments, the binders can be present in an amount of about 20% by weight to about 90% by weight, e.g., about 25% to about 75% by weight, e.g., about 30% to about 60% by weight, based on the total weight of the edible, orally dissolvable film. Where the binder comprises a combination of components, the components can be present in varying ratios with respect to one another. In one embodiment, the binder comprises a majority of a modified cellulose (i.e., at least about 50% of the binder component comprises one or more modified celluloses) and a minority of one or more other binders.

### Plasticizers

Plasticizers are typically incorporated to promote softness and/or flexibility of the edible, orally dissolvable films provided herein. Without being limited by theory, it is believed the plasticizer can act to facilitate hydration within the film. Examples of suitable plasticizers include, but are not limited to, organic non-polymeric materials. Certain non-limiting plasticizers comprise glyceryl monostearate, triethyl citrate, glycerin, polyethylene glycol, propylene glycol, and combinations thereof. In various embodiments, the plasticizer is selected from the group consisting of glycerin, propylene glycol, and combinations thereof.

The amount of plasticizer utilized within the edible, orally dissolvable film can vary. The amount of plasticizer present within a representative edible, orally dissolvable film can be at least about 1 percent, at least about 2 percent, at least about 3 percent, at least about 5 percent, at least about 10 percent, or at least about 15 percent of the final formed product, on a dry weight basis. When employed, the amount of plasticizer employed within a representative processed oral product typically is about 50 percent or less, about 35 percent or less, about 25 percent or less, 15 percent or less, about 10 percent or less, or about 5 percent or less, of the film.

### Active Ingredients

A film as disclosed herein typically includes one or more active ingredients (although, in some embodiments, a film may be provided that does not include an active ingredient, e.g., where it is used as a component of a product that incorporates an active ingredient in another component thereof).

As used herein, an “active ingredient” refers to one or more substances belonging to any of the following categories: API (active pharmaceutical ingredient), food additives, natural medicaments, and naturally occurring substances that can have an effect on humans. Example active ingredients include any ingredient known to impact one or more biological functions within the body, such as ingredients that furnish pharmacological activity or other direct effect in the diagnosis, cure, mitigation, treatment, or prevention of disease, or which affect the structure or any function of the body of humans (e.g., provide a stimulating action on the central nervous system, have an energizing effect, an antipyretic or analgesic action, or an otherwise useful effect on the body). In some embodiments, the active ingredient may be of the type generally referred to as dietary supplements, nutraceuticals, “phytochemicals” or “functional foods.” These types of additives are sometimes defined in the art as encompassing substances typically available from naturally-occurring sources (e.g., botanical materials) that provide one or more advantageous biological effects (e.g., health promotion, disease prevention, or other medicinal properties), but are not classified or regulated as drugs.

Non-limiting examples of active ingredients include those falling in the categories of botanical ingredients, stimulants, amino acids, nicotine components, and/or pharmaceutical, nutraceutical, and medicinal ingredients (e.g., vitamins, such as A, B3, B6, B12, and C, and/or cannabinoids, such as tetrahydrocannabinol (THC) and cannabidiol (CBD)). Each of these categories is further described herein below. The particular choice of active ingredients will vary depending upon the desired flavor, texture, and desired characteristics of the particular film and/or oral product.

In certain embodiments, the active ingredient is selected from the group consisting of caffeine, taurine, GABA, theanine, vitamin C, lemon balm extract, ginseng, citicoline, sunflower lecithin, and combinations thereof. For example, the active ingredient can include a combination of caffeine, theanine, and optionally ginseng. In another embodiment, the active ingredient includes a combination of theanine, gamma-amino butyric acid (GABA), and lemon balm extract. In a further embodiment, the active ingredient includes theanine, theanine and tryptophan, or theanine and one or more B vitamins (e.g., vitamin B6 or B12). In a still further embodiment, the active ingredient includes a combination of caffeine, taurine, and vitamin C.

Furthermore, any of the types of active ingredients described herein may be encapsulated in the composition, the final product, or both to avoid chemical degradation (e.g., where the active ingredient is sensitive to oxidative, photolytic, thermal, or evaporative degradation during processing or upon storage of the oral product) or to reduce strong taste of these actives, including but not limited to caffeine, Vitamin A, and iron (Fe). Additionally, these encapsulated actives may need to be paired with an excipient in the composition to increase their solubility and/or bioavailability. Non-limiting examples of these excipients include beta-carotene, lycopene, Vitamin D, Vitamin E, Co-enzyme Q10, Vitamin K, and curcumin.

The particular percentages of active ingredients present will vary depending upon the desired characteristics of the particular product. Typically, an active ingredient or combination thereof is present in a total concentration of at least about 0.001% by weight of the composition, such as in a range from about 0.001% to about 20%. In some embodiments, the active ingredient or combination of active ingredients is present in a concentration from about 0.1% w/w to about 10% by weight, such as, e.g., from about 0.5% w/w to about 10%, from about 1% to about 10%, from about 1% to about 5% by weight, based on the total weight of the composition. In some embodiments, the active ingredient or combination of active ingredients is present in a concentration of from about 0.001%, about 0.01%, about 0.1% , or about 1%, up to about 20% by weight, such as, e.g., from about 0.001%, about 0.002%, about 0.003%, about 0.004%, about 0.005%, about 0.006%, about 0.007%, about 0.008%, about 0.009%, about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5% about 0.6%, about 0.7%, about 0.8%, or about 0.9%, to about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20% by weight, based on the total weight of the composition. Further suitable ranges for specific active ingredients are provided herein below.

### Botanical

In some embodiments, the active ingredient comprises a botanical ingredient. As used herein, the term “botanical ingredient” or “botanical” refers to any plant material or fungal-derived material, including plant material in its natural form and plant material derived from natural plant materials, such as extracts or isolates from plant materials or treated plant materials (e.g., plant materials subjected to heat treatment, fermentation, bleaching, or other treatment processes capable of altering the physical and/or chemical nature of the material). For the purposes of the present disclosure, a “botanical” includes, but is not limited to, “herbal materials,” which refer to seed-producing plants that do not develop persistent woody tissue and are often valued for their medicinal or sensory characteristics (e.g., teas or tisanes). Reference to botanical material as “non-tobacco” is intended to exclude tobacco materials (i.e., does not include any Nicotiana species). In some embodiments, the compositions as disclosed herein can be characterized as free of any tobacco material (e.g., any embodiment as disclosed herein may be completely or substantially free of any tobacco material). By “substantially free” is meant that no tobacco material has been intentionally added. For example, certain embodiments can be characterized as having less than 0.001% by weight of tobacco, or less than 0.0001%, or even 0% by weight of tobacco.

When present, a botanical is typically at a concentration of from about 0.01% w/w to about 10% by weight, such as, e.g., from about 0.01% w/w, about 0.05%, about 0.1%, or about 0.5%, to about 1%, about 2%, about 3%, about 4%, about 5%, about 6%, about 7%, about 8%, about 9%, or about 10%, about 11%, about 12%, about 13%, about 14%, or about 15% by weight, based on the total weight of the orally dissolving film or based on the total weight of the oral product comprising the orally dissolving film.

The botanical materials useful in the present disclosure may comprise, without limitation, any of the compounds and sources set forth herein, including mixtures thereof. Certain botanical materials of this type are sometimes referred to as dietary supplements, nutraceuticals, “phytochemicals” or “functional foods.” Certain botanicals, as the plant material or an extract thereof, have found use in traditional herbal medicine, and are described further herein. Non-limiting examples of botanicals or botanical-derived materials include ashwagandha, Bacopa monniera, baobab, basil, Centella asiatica, Chai-hu, chamomile, cherry blossom, chlorophyll, cinnamon, citrus, cloves, cocoa, cordyceps, curcumin, damiana, Dorstenia arifolia, Dorstenia odorata, essential oils, eucalyptus, fennel, Galphimia glauca, ginger, Ginkgo biloba, ginseng (e.g., Panax ginseng), green tea, Griffonia simplicifolia, guarana, cannabis, hemp, hops, jasmine, Kaempferia parviflora (Thai ginseng), kava, lavender, lemon balm, lemongrass, licorice, lutein, maca, matcha, Nardostachys chinensis, oil-based extract of Viola odorata, peppermint, quercetin, resveratrol, Rhizoma gastrodiae, Rhodiola, rooibos, rose essential oil, rosemary, Sceletium tortuosum, Schisandra, Skullcap, spearmint extract, Spikenard, terpenes, tisanes, turmeric, Turnera aphrodisiaca, valerian, white mulberry, and Yerba mate.

In some embodiments, the active ingredient comprises lemon balm. Lemon balm (Melissa officinalis) is a mildly lemon-scented herb from the same family as mint (Lamiaceae). The herb is native to Europe, North Africa, and West Asia. The tea of lemon balm, as well as the essential oil and the extract, are used in traditional and alternative medicine. In some embodiments, the active ingredient comprises lemon balm extract. In some embodiments, the lemon balm extract is present in an amount of from about 1 to about 4% by weight, based on the total weight of the orally dissolving film or based on the total weight of the oral product comprising the orally dissolving film.

In some embodiments, the active ingredient comprises ginseng. Ginseng is the root of plants of the genus Panax, which are characterized by the presence of unique steroid saponin phytochemicals (ginsenosides) and gintonin. Ginseng finds use as a dietary supplement in energy drinks or herbal teas, and in traditional medicine. Cultivated species include Korean ginseng (P. ginseng), South China ginseng (P. notoginseng), and American ginseng (P. quinquefolius). American ginseng and Korean ginseng vary in the type and quantity of various ginsenosides present. In some embodiments, the ginseng is American ginseng or Korean ginseng. In specific embodiments, the active ingredient comprises Korean ginseng. In some embodiments, ginseng is present in an amount of from about 0.4 to about 0.6% by weight, based on the total weight of the orally dissolving film or based on the total weight of the oral product comprising the orally dissolving film.

### Stimulants

In some embodiments, the active ingredient comprises one or more stimulants. As used herein, the term “stimulant” refers to a material that increases activity of the central nervous system and/or the body, for example, enhancing focus, cognition, vigor, mood, alertness, and the like. Non-limiting examples of stimulants include caffeine, theacrine, theobromine, and theophylline. Theacrine (1,3,7,9-tetramethyluric acid) is a purine alkaloid which is structurally related to caffeine, and possesses stimulant, analgesic, and anti-inflammatory effects. Present stimulants may be natural, naturally derived, or wholly synthetic. For example, certain botanical materials (guarana, tea, coffee, cocoa, and the like) may possess a stimulant effect by virtue of the presence of e.g., caffeine or related alkaloids, and accordingly are “natural” stimulants. By “naturally derived” is meant the stimulant (e.g., caffeine, theacrine) is in a purified form, outside its natural (e.g., botanical) matrix. For example, caffeine can be obtained by extraction and purification from botanical sources (e.g., tea). By “wholly synthetic”, it is meant that the stimulant has been obtained by chemical synthesis. In some embodiments, the active ingredient comprises caffeine. In some embodiments, the caffeine is present in an encapsulated form. On example of an encapsulated caffeine is Vitashure , available from Balchem Corp., 52 Sunrise Park Road, New Hampton, N.Y., 10958.

When present, a stimulant or combination of stimulants (e.g., caffeine, theacrine, and combinations thereof) is typically at a concentration of from about 0.1% w/w to about 15% by weight, such as, e.g., from about 0.1% w/w, about 0.2%, about 0.3%, about 0.4%, about 0.5% about 0.6%, about 0.7%, about 0.8%, or about 0.9%, to about 1%, about 2%, about 3%, about 4%, about 5%, about 6%, about 7%, about 8%, about 9%, about 10%, about 11%, about 12%, about 13%, about 14%, or about 15% by weight, based on the total weight of the composition. In some embodiments, the composition comprises caffeine in an amount of from about 1.5 to about 6% by weight, based on the total weight of the orally dissolving film or based on the total weight of the oral product comprising the orally dissolving film.

Amino acids

In some embodiments, the active ingredient comprises an amino acid. As used herein, the term “amino acid” refers to an organic compound that contains amine (—NH₂) and carboxyl (—COOH) or sulfonic acid (SO₃H) functional groups, along with a side chain (R group), which is specific to each amino acid. Amino acids may be proteinogenic or non-proteinogenic. By “proteinogenic” is meant that the amino acid is one of the twenty naturally occurring amino acids found in proteins. The proteinogenic amino acids include alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine. By “non-proteinogenic” is meant that either the amino acid is not found naturally in protein, or is not directly produced by cellular machinery (e.g., is the product of post-translational modification). Non-limiting examples of non-proteinogenic amino acids include gamma-aminobutyric acid (GABA), taurine (2-aminoethanesulfonic acid), theanine (L-γ-glutamylethylamide), hydroxyproline, and beta-alanine. In some embodiments, the active ingredient comprises theanine. In some embodiments, the active ingredient comprises GABA. In some embodiments, the active ingredient comprises a combination of theanine and GABA. In some embodiments, the active ingredient is a combination of theanine, GABA, and lemon balm. In some embodiments, the active ingredient is a combination of caffeine, theanine, and ginseng. In some embodiments, the active ingredient comprises taurine. In some embodiments, the active ingredient is a combination of caffeine and taurine.

When present, an amino acid or combination of amino acids (e.g., theanine, GABA, and combinations thereof) is typically at a concentration of from about 0.1% w/w to about 15% by weight, such as, e.g., from about 0.1% w/w, about 0.2%, about 0.3%, about 0.4%, about 0.5% about 0.6%, about 0.7%, about 0.8%, or about 0.9%, to about 1%, about 2%, about 3%, about 4%, about 5%, about 6%, about 7%, about 8%, about 9%, about 10%, about 11%, about 12%, about 13%, about 14%, or about 15% by weight, based on the total weight of the orally dissolving film or based on the total weight of the oral product comprising the orally dissolving film.

### Vitamins

In some embodiments, the active ingredient comprises a vitamin or combination of vitamins. As used herein, the term “vitamin” refers to an organic molecule (or related set of molecules) that is an essential micronutrient needed for the proper functioning of metabolism in a mammal. There are thirteen vitamins required by human metabolism, which are: vitamin A (as all-trans-retinol, all-trans-retinyl-esters, as well as all-trans-beta-carotene and other provitamin A carotenoids), vitamin B1 (thiamine), vitamin B2 (riboflavin), vitamin B3 (niacin), vitamin B5 (pantothenic acid), vitamin B6 (pyridoxine), vitamin B7 (biotin), vitamin B9 (folic acid or folate), vitamin B12 (cobalamins), vitamin C (ascorbic acid), vitamin D (calciferols), vitamin E (tocopherols and tocotrienols), and vitamin K (quinones). In some embodiments, the active ingredient comprises vitamin C. In some embodiments, the active ingredient is a combination of vitamin C, caffeine, and taurine.

When present, a vitamin or combination of vitamins (e.g., vitamin B6, vitamin B12, vitamin E, vitamin C, or a combination thereof) is typically at a concentration of from about 0.01% w/w to about 6% by weight, such as, e.g., from about 0.01%, about 0.02%, about 0.03%, about 0.04%, about 0.05%, about 0.06%, about 0.07%, about 0.08%, about 0.09%, or about 0.1% w/w, to about 0.2%, about 0.3%, about 0.4%, about 0.5% about 0.6%, about 0.7%, about 0.8%, about 0.9%, about 1%, about 2%, about 3%, about 4%, about 5% , or about 6% by weight, based on the total weight of the orally dissolving film or based on the total weight of the oral product comprising the orally dissolving film.

### Antioxidants

In some embodiments, the active ingredient comprises one or more antioxidants. As used herein, the term “antioxidant” refers to a substance which prevents or suppresses oxidation by terminating free radical reactions, and may delay or prevent some types of cellular damage. Antioxidants may be naturally occurring or synthetic. Naturally occurring antioxidants include those found in foods and botanical materials. Non-limiting examples of antioxidants include certain botanical materials, vitamins, polyphenols, and phenol derivatives.

Examples of botanical materials which are associated with antioxidant characteristics include without limitation acai berry, alfalfa, allspice, annatto seed, apricot oil, basil, bee balm, wild bergamot, black pepper, blueberries, borage seed oil, bugleweed, cacao, calamus root, catnip, catuaba, cayenne pepper, chaga mushroom, chervil, cinnamon, dark chocolate, potato peel, grape seed, ginseng, gingko biloba, Saint John's Wort, saw palmetto, green tea, black tea, black cohosh, cayenne, chamomile, cloves, cocoa powder, cranberry, dandelion, grapefruit, honeybush, echinacea, garlic, evening primrose, feverfew, ginger, goldenseal, hawthorn, hibiscus flower, jiaogulan, kava, lavender, licorice, marjoram, milk thistle, mints (menthe), oolong tea, beet root, orange, oregano, papaya, pennyroyal, peppermint, red clover, rooibos (red or green), rosehip, rosemary, sage, clary sage, savory, spearmint, spirulina, slippery elm bark, sorghum bran hi-tannin, sorghum grain hi-tannin, sumac bran, comfrey leaf and root, goji berries, gutu kola, thyme, turmeric, uva ursi, valerian, wild yam root, wintergreen, yacon root, yellow dock, yerba mate, yerba santa, bacopa monniera, withania somnifera, Lion's mane, and silybum marianum. Such botanical materials may be provided in fresh or dry form, essential oils, or may be in the form of an extracts. The botanical materials (as well as their extracts) often include compounds from various classes known to provide antioxidant effects, such as minerals, vitamins, isoflavones, phytoesterols, allyl sulfides, dithiolthiones, isothiocyanates, indoles, lignans, flavonoids, polyphenols, and carotenoids. Examples of compounds found in botanical extracts or oils include ascorbic acid, peanut endocarb, resveratrol, sulforaphane, beta-carotene, lycopene, lutein, co-enzyme Q, carnitine, quercetin, kaempferol, and the like. See, e.g., Santhosh et al., Phytomedicine, 12(2005) 216-220, which is incorporated herein by reference.

Non-limiting examples of other suitable antioxidants include citric acid, Vitamin E or a derivative thereof, a tocopherol, epicatechol, epigallocatechol, epigallocatechol gallate, erythorbic acid, sodium erythorbate, 4-hexylresorcinol, theaflavin, theaflavin monogallate A or B, theaflavin digallate, phenolic acids, glycosides, quercitrin, isoquercitrin, hyperoside, polyphenols, catechols, resveratrols, oleuropein, butylated hydroxyanisole (BHA), butylated hydroxytoluene (BHT), tertiary butylhydroquinone (TBHQ), and combinations thereof.

When present, an antioxidant is typically at a concentration of from about 0.001% w/w to about 10% by weight, such as, e.g., from about 0.001%, about 0.005%, about 0.01% w/w, about 0.05%, about 0.1%, or about 0.5%, to about 1%, about 2%, about 3%, about 4%, about 5%, about 6%, about 7%, about 8%, about 9%, or about 10%, based on the total weight of the orally dissolving film or based on the total weight of the oral product comprising the orally dissolving film.

### Nicotine Component

In certain embodiments, the films or oral products of the present disclosure can include a nicotinic compound. Various nicotinic compounds, and methods for their administration, are set forth in US Pat. Pub. No. 2011/0274628 to Borschke, which is incorporated herein by reference. As used herein, “nicotinic compound” or “source of nicotine” often refers to naturally-occurring or synthetic nicotinic compound unbound from a plant material, meaning the compound is at least partially purified and not contained within a plant structure, such as a tobacco leaf. Most preferably, nicotine is naturally-occurring and obtained as an extract from a Nicotiana species (e.g., tobacco). The nicotine can have the enantiomeric form S(−)-nicotine, R(+)-nicotine, or a mixture of S(−)-nicotine and R(+)-nicotine. Most preferably, the nicotine is in the form of S(−)-nicotine (e.g., in a form that is virtually all S(−)-nicotine) or a racemic mixture composed primarily or predominantly of S(−)-nicotine (e.g., a mixture composed of about 95 weight parts S(−)-nicotine and about 5 weight parts R(+)-nicotine). Most preferably, the nicotine is employed in virtually pure form or in an essentially pure form. Highly preferred nicotine that is employed has a purity of greater than about 95 percent, more preferably greater than about 98 percent, and most preferably greater than about 99 percent, on a weight basis.

In certain embodiments, a nicotine component may be included in free base form, salt form, as a complex, or as a solvate. By “nicotine component” is meant any suitable form of nicotine (e.g., free base or salt) for providing oral absorption of at least a portion of the nicotine present. Typically, the nicotine component is selected from the group consisting of nicotine free base and a nicotine salt. In some embodiments, nicotine is in its free base form, which easily can be adsorbed in for example, a microcrystalline cellulose material to form a microcrystalline cellulose-nicotine carrier complex. See, for example, the discussion of nicotine in free base form in US Pat. Pub. No. 2004/0191322 to Hansson, which is incorporated herein by reference.

In some embodiments, at least a portion of the nicotine can be employed in the form of a salt. Salts of nicotine can be provided using the types of ingredients and techniques set forth in U.S. Pat. No. 2,033,909 to Cox et al. and Perfetti, Beitrage Tabakforschung Int., 12: 43-54 (1983), which are incorporated herein by reference. Additionally, salts of nicotine are available from sources such as Pfaltz and Bauer, Inc. and K&K Laboratories, Division of ICN Biochemicals, Inc. Typically, the nicotine component is selected from the group consisting of nicotine free base, a nicotine salt such as hydrochloride, dihydrochloride, monotartrate, bitartrate, sulfate, salicylate, and nicotine zinc chloride. In some embodiments, the nicotine component or a portion thereof is a nicotine salt with one or more organic acids.

For customer satisfaction, in some embodiments, it may be desirable to provide a basic amine-containing oral product configured for oral use which retains the initial basic amine content (e.g., nicotine content) during storage, and which delivers substantially the full amount of basic amine (e.g., nicotine) initially present in the oral product. In some such embodiments, nicotine or other basic amine is employed in association with at least a portion of an organic acid or an alkali metal salt thereof (referred to herein as “ion pairing”). Embodiments of the films disclosed herein can comprise at least one binder, a plasticizer, a basic amine, such as nicotine or a nicotine component; water; and an organic acid, an alkali metal salt of an organic acid, or a combination thereof, wherein the organic acid has a logP value of from about 1.4 to about 8.0.

As disclosed herein, at least a portion of the basic amine (e.g., nicotine) is associated with at least a portion of the organic acid or the alkali metal salt thereof. It is noted that for the purposes of the present disclosure, the basic amine can be included in place of or in addition to other active ingredients described in more detail herein. Depending on multiple variables (concentration, pH, nature of the organic acid, and the like), the basic amine present in the composition can exist in multiple forms, including ion paired, in solution (i.e., fully solvated), as the free base, as a cation, as a salt, or any combination thereof. The relative amounts of the various components within the oral product composition may vary, and typically are selected so as to provide the desired sensory and performance characteristics to the oral product. In some embodiments, the association between the basic amine and at least a portion of the organic acid or the alkali metal salt thereof is in the form of an ion pair between the basic amine and a conjugate base of the organic acid.

Ion pairing describes the partial association of oppositely charged ions in relatively concentrated solutions to form distinct chemical species called ion pairs. The strength of the association (i.e., the ion pairing) depends on the electrostatic force of attraction between the positive and negative ions (i.e., a protonated basic amine such as nicotine, and the conjugate base of the organic acid). By “conjugate base” is meant the base resulting from deprotonation of the corresponding acid (e.g., benzoate is the conjugate base of benzoic acid). On average, a certain population of these ion pairs exists at any given time, although the formation and dissociation of ion pairs is continuous. In the oral products as disclosed herein, and/or upon oral use of said oral products (e.g., upon contact with saliva), the basic amine, for example nicotine, and the conjugate base of the organic acid exist at least partially in the form of an ion pair. Without wishing to be bound by theory, it is believed that such ion pairing may minimize chemical degradation of the basic amine and/or enhance the oral availability of the basic amine (e.g., nicotine). At alkaline pH values (e.g., such as from about 7.5 to about 9), certain basic amines, for example nicotine, are largely present in the free base form, which has relatively low water solubility, and low stability with respect to evaporation and oxidative decomposition, but high mucosal availability. Conversely, at acidic pH values (such as from about 6.5 to about 4), certain basic amines, for example nicotine, are largely present in a protonated form, which has relatively high water solubility, and higher stability with respect to evaporation and oxidative decomposition, but low mucosal availability. Surprisingly, according to the present disclosure, it has been found that the properties of stability, solubility, and availability of the nicotine in a composition configured for oral use can be mutually enhanced through ion pairing or salt formation of nicotine with appropriate organic acids and/or their conjugate bases. Specifically, nicotine-organic acid ion pairs of moderate lipophilicity result in favorable stability and absorption properties. Lipophilicity is conveniently measured in terms of logP, the partition coefficient of a molecule between a lipophilic phase and an aqueous phase, usually octanol and water, respectively. An octanol-water partitioning favoring distribution of a basic amine-organic acid ion pair into octanol is predictive of good absorption of the basic amine present in the composition through the oral mucosa.

As noted above, at alkaline pH values (e.g., such as from about 7.5 to about 9), nicotine is largely present in the free base form (and accordingly, a high partitioning into octanol), while at acidic pH values (such as from about 6.5 to about 4), nicotine is largely present in a protonated form (and accordingly, a low partitioning into octanol). An ion pair between certain organic acids (e.g., having a logP value of from about 1.4 to about 8.0. such as from about 1.4 to about 4.5, allows nicotine partitioning into octanol consistent with that predicted for nicotine partitioning into octanol at a pH of 8.4.

One of skill in the art will recognize that the extent of ion pairing in the disclosed composition, both before and during use by the consumer, may vary based on, for example, pH, the nature of the organic acid, the concentration of nicotine, the concentration of the organic acid or conjugate base of the organic acid present in the composition, the moisture content of the composition, the ionic strength of the composition, and the like. One of skill in the art will also recognize that ion pairing is an equilibrium process influenced by the foregoing variables. Accordingly, quantification of the extent of ion pairing is difficult or impossible by calculation or direct observation. However, as disclosed herein, the presence of ion pairing may be demonstrated through surrogate measures such as partitioning of the nicotine between octanol and water or membrane permeation of aqueous solutions of the basic amine plus organic acids and/or their conjugate bases.

### Organic Acid

As used herein, the term “organic acid” refers to an organic (i.e., carbon-based) compound that is characterized by acidic properties. Typically, organic acids are relatively weak acids (i.e., they do not dissociate completely in the presence of water), such as carboxylic acids (—CO₂H) or sulfonic acids (—SO₂OH). As used herein, reference to organic acid means an organic acid that is intentionally added. In this regard, an organic acid may be intentionally added as a specific composition ingredient as opposed to merely being inherently present as a component of another composition ingredient (e.g., the small amount of organic acid which may inherently be present in a composition ingredient, such as a tobacco material).

Suitable organic acids will typically have a range of lipophilicities (i.e., a polarity giving an appropriate balance of water and organic solubility). Typically, lipophilicities of suitable organic acids, as indicated by logP, will vary between about 1 and about 12 (more soluble in octanol than in water). In some embodiments, the organic acid has a logP value from about 1 to about 12, e.g., from about 3.0, about 3.5, about 4.0, about 4.5, about 5.0, about 5.5, about 6.0, about 6.5, about 7.0, about 7.5, or about 8.0, to about 8.5, about 9.0, about 9.5, about 10.0, about 10.5, about 11.0, about 11.5, or about 12.0. In certain embodiments, lipophilicities of suitable organic acids, as indicated by logP, will vary between about 1.4 and about 4.5 (more soluble in octanol than in water). In some embodiments, the organic acid has a logP value of from about 1.5 to about 4.0, e.g., from about 1.5, about 2.0, about 2.5, or about 3.0, to about 3.5, about 4.0, about 4.5, or about 5.0. Particularly suitable organic acids have a logP value of from about 1.7 to about 4, such as from about 2.0, about 2.5, or about 3.0, to about 3.5, or about 4.0. In specific embodiments, the organic acid has a logP value of about 2.5 to about 3.5. In some embodiments, organic acids outside this range may also be utilized for various purposes and in various amounts, as described further herein below. For example, in some embodiments, the organic acid may have a logP value of greater than about 4.5, such as from about 4.5 to about 12.0. Particularly, the presence of certain solvents or solubilizing agents (e.g., inclusion in the composition of glycerin or propylene glycol) may extend the range of lipophilicity (i.e., values of logP higher than 4.5, such as from about 4.5 to about 12.0).

Without wishing to be bound by theory, it is believed that moderately lipophilic organic acids (e.g., logP of from about 1.4 to about 4.5) produce ion pairs with nicotine which are of a polarity providing good octanol-water partitioning of the ion pair, and hence partitioning of nicotine, into octanol versus water. As discussed above, such partitioning into octanol is predictive of favorable oral availability.

In some embodiments, the organic acid is a carboxylic acid or a sulfonic acid. The carboxylic acid or sulfonic acid functional group may be attached to any alkyl, cycloalkyl, heterocycloalkyl, aryl, or heteroaryl group having, for example, from one to twenty carbon atoms (C₁-C₂₀). In some embodiments, the organic acid is an alkyl, cycloalkyl, heterocycloalkyl, aryl, or heteroaryl carboxylic or sulfonic acid.

As used herein, “alkyl” refers to any straight chain or branched chain hydrocarbon. The alkyl group may be saturated (i.e., having all sp³ carbon atoms), or may be unsaturated (i.e., having at least one site of unsaturation). As used herein, the term “unsaturated” refers to the presence of a carbon-carbon, sp² double bond in one or more positions within the alkyl group. Unsaturated alkyl groups may be mono- or polyunsaturated. Representative straight chain alkyl groups include, but are not limited to, methyl, ethyl, n-propyl, n-butyl, n-pentyl, and n-hexyl. Branched chain alkyl groups include, but are not limited to, isopropyl, sec-butyl, isobutyl, tert-butyl, isopentyl, and 2-methylbutyl. Representative unsaturated alkyl groups include, but are not limited to, ethylene or vinyl, allyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like. An alkyl group can be unsubstituted or substituted.

“Cycloalkyl” as used herein refers to a carbocyclic group, which may be mono- or bicyclic. Cycloalkyl groups include rings having 3 to 7 carbon atoms as a monocycle or 7 to 12 carbon atoms as a bicycle. Examples of monocyclic cycloalkyl groups include cyclopropyl, cyclobutyl, cyclopentyl, cyclohexyl, cycloheptyl, and cyclooctyl. A cycloalkyl group can be unsubstituted or substituted, and may include one or more sites of unsaturation (e.g., cyclopentenyl or cyclohexenyl).

The term “aryl” as used herein refers to a carbocyclic aromatic group. Examples of aryl groups include, but are not limited to, phenyl and naphthyl. An aryl group can be unsubstituted or substituted.

“Heteroaryl” and “heterocycloalkyl” as used herein refer to an aromatic or non-aromatic ring system, respectively, in which one or more ring atoms is a heteroatom, e.g. nitrogen, oxygen, and sulfur.

The heteroaryl or heterocycloalkyl group comprises up to 20 carbon atoms and from 1 to 3 heteroatoms selected from N, O, and S. A heteroaryl or heterocycloalkyl may be a monocycle having 3 to 7 ring members (for example, 2 to 6 carbon atoms and 1 to 3 heteroatoms selected from N, O, and S) or a bicycle having 7 to 10 ring members (for example, 4 to 9 carbon atoms and 1 to 3 heteroatoms selected from N, O, and S), for example: a bicyclo[4,5], [5,5], [5,6], or [6,6] system. Examples of heteroaryl groups include by way of example and not limitation, pyridyl, thiazolyl, tetrahydrothiophenyl, pyrimidinyl, furanyl, thienyl, pyrrolyl, pyrazolyl, imidazolyl, tetrazolyl, benzofuranyl, thianaphthalenyl, indolyl, indolenyl, quinolinyl, isoquinolinyl, benzimidazolyl, isoxazolyl, pyrazinyl, pyridazinyl, indolizinyl, isoindolyl, 3H-indolyl, 1H-indazolyl, purinyl, 4H-quinolizinyl, phthalazinyl, naphthyridinyl, quinoxalinyl, quinazolinyl, cinnolinyl, pteridinyl, 4aH-carbazolyl, carbazolyl, phenanthridinyl, acridinyl, pyrimidinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, benzotriazolyl, benzisoxazolyl, and isatinoyl. Examples of heterocycloalkyls include by way of example and not limitation, dihydroypyridyl, tetrahydropyridyl (piperidyl), tetrahydrothiophenyl, piperidinyl, 4-piperidonyl, pyrrolidinyl, 2-pyrrolidonyl, tetrahydrofuranyl, tetrahydropyranyl, bis-tetrahydropyranyl, tetrahydroquinolinyl, tetrahydroisoquinolinyl, decahydroquinolinyl, octahydroisoquinolinyl, piperazinyl, quinuclidinyl, and morpholinyl. Heteroaryl and heterocycloalkyl groups can be unsubstituted or substituted.

“Substituted” as used herein and as applied to any of the above alkyl, aryl, cycloalkyl, heteroaryl, heterocyclyl, means that one or more hydrogen atoms are each independently replaced with a substituent. Typical substituents include, but are not limited to, —Cl, Br, F, alkyl, —OH, —OCH₃, NH₂, —NHCH₃, —N(CH₃)₂, —CN, —NC(═O)CH₃, —C(═O)—, —C(═O)NH₂, and —C(═O)N(CH₃)₂. Wherever a group is described as “optionally substituted,” that group can be substituted with one or more of the above substituents, independently selected for each occasion. In some embodiments, the substituent may be one or more methyl groups or one or more hydroxyl groups.

In some embodiments, the organic acid is an alkyl carboxylic acid. Non-limiting examples of alkyl carboxylic acids include formic acid, acetic acid, propionic acid, butyric acid, valeric acid, caproic acid, heptanoic acid, octanoic acid, nonanoic acid, decanoic acid, undecanoic acid, dodecanoic acid, stearic acid, oleic acid, linoleic acid, linolenic acid, and the like.

In some embodiments, the organic acid is an alkyl sulfonic acid. Non-limiting examples of alkyl sulfonic acids include propanesulfonic acid, heptanesulfonic acid, and octanesulfonic acid.

In some embodiments, the alkyl carboxylic or sulfonic acid is substituted with one or more hydroxyl groups. Non-limiting examples include glycolic acid, 4-hydroxybutyric acid, and lactic acid.

In some embodiments, an organic acid may include more than one carboxylic acid group or more than one sulfonic acid group (e.g., two, three, or more carboxylic acid groups). Non-limiting examples include oxalic acid, fumaric acid, maleic acid, and glutaric acid. In organic acids containing multiple carboxylic acids (e.g., from two to four carboxylic acid groups), one or more of the carboxylic acid groups may be esterified. Non-limiting examples include succinic acid monoethyl ester, monomethyl fumarate, monomethyl or dimethyl citrate, and the like.

In some embodiments, the organic acid may include more than one carboxylic acid group and one or more hydroxyl groups. Non-limiting examples of such acids include tartaric acid, citric acid, and the like.

In some embodiments, the organic acid is an aryl carboxylic acid or an aryl sulfonic acid. Non-limiting examples of aryl carboxylic and sulfonic acids include benzoic acid, toluic acids, salicylic acid, benzenesulfonic acid, and p-toluenesulfonic acid.

Further non-limiting examples of organic acids which may be useful in certain embodiments include 2,2-dichloroacetic acid, 2-hydroxyethanesulfonic acid, 2-oxoglutaric acid, 4-acetamidobenzoic acid, 4-aminosalicylic acid, adipic acid, ascorbic acid (L), aspartic acid (L), alpha-methylbutyric acid, camphoric acid (+), camphor-10-sulfonic acid (+), cinnamic acid, cyclamic acid, dodecylsulfuric acid, ethane-1,2-disulfonic acid, ethanesulfonic acid, furoic acid, galactaric acid, gentisic acid, glucoheptonic acid, gluconic acid, glucuronic acid, glutamic acid, glycerophosphoric acid, glycolic acid, hippuric acid, isobutyric acid, isovaleric acid, lactobionic acid, lauric acid, levulinic acid, malic acid, malonic acid, mandelic acid, methanesulfonic acid, naphthalene-1,5-disulfonic acid, naphthalene-2-sulfonic acid, oleic acid, palmitic acid, pamoic acid, phenylacetic acid, pyroglutamic acid, pyruvic acid, sebacic acid, stearic acid, and undecylenic acid.

Examples of suitable acids include, but are not limited to, the list of organic acids in Table 1.

TABLE 1
Non-limiting examples of suitable organic acids

| Acid Name | log(P) |
|---|---|
| benzoic acid | 1.9 |
| phenylacetic | 1.4 |
| p-toluic acid | 2.3 |
| ethyl benzoic acid | 2.9 |
| isopropyl benzoic acid | 3.5 |
| 4-phenylbutyric | 2.4 |
| 2-napthoxyacetic acid | 2.5 |
| napthylacetic acid | 2.7 |
| heptanoic acid | 2.5 |
| octanoic acid | 3.05 |
| nonanoic acid | 3.5 |
| decanoic acid | 4.09 |
| 9-deceneoic acid | 3.3 |
| 2-deceneoic acid | 3.8 |
| 10-undecenoic acid | 3.9 |
| dodecandioic acid | 3.2 |
| dodecanoic acid | 4.6 |
| myristic acid | 5.3 |
| palmitic acid | 6.4 |
| stearic acid | 7.6 |
| cyclohexanebutanoic acid | 3.4 |
| 1-heptane sulfonic acid | 2.0 |
| 1-octane sulfonic acid | 2.5 |
| 1-nonane sulfonic acid | 3.1 |
| monooctyl succinate | 2.8 |
| tocopherol succinate | 10.2 |
| monomenthyl succinate | 3 |
| monomenthyl glutarate | 3.4 |
| norbixin | 7.2 |
| ((2E,4E,6E,8E,10E,12E,14E,16E,18E)- | |
| 4,8,13,17-tetramethylicosa- | |
| 2,4,6,8,10,12,14,16,18-nonaenedioic acid) | |
| bixin | 7.5 |
| ((2E,4E,6E,8E,10E,12E,14E,16Z,18E)-20- | |
| methoxy-4,8,13,17-tetramethyl-20- | |
| oxoicosa-2,4,6,8,10,12,14,16,18- | |
| nonaenoic acid) | |

In some embodiments, the organic acid is benzoic acid, a toluic acid, benzenesulfonic acid, toluenesulfonic acid, hexanoic acid, heptanoic acid, decanoic acid, or octanoic acid. In some embodiments, the organic acid is benzoic acid, octanoic acid, or decanoic acid. In some embodiments, the organic acid is octanoic acid.

In some embodiments, the organic acid is a mono ester of a di- or poly-acid, such as mono-octyl succinate, mono-octyl fumarate, or the like. For example, the organic acid is a mono ester of a dicarboxylic acid or a poly-carboxylic acid. In some embodiments, the dicarboxylic acid is malonic acid, succinic acid, glutaric acid, adipic acid, fumaric acid, maleic acid, or a combination thereof. In some embodiments, the dicarboxylic acid is succinic acid, glutaric acid, fumaric acid, maleic acid, or a combination thereof. In some embodiments, the dicarboxylic acid is succinic acid, glutaric acid, or a combination thereof.

In some embodiments, the alcohol forming the mono ester of the dicarboxylic acid is a lipophilic alcohol. Examples of suitable lipophilic alcohols include, but are not limited to, octanol, menthol, and tocopherol. In some embodiments, the organic acid is an octyl mono ester of a dicarboxylic acid, such as monooctyl succinate, monooctyl fumarate, or the like. In some embodiments, the organic acid is a monomenthyl ester of a dicarboxylic acid. Certain menthyl esters may be desirable in oral compositions as described herein by virtue of the cooling sensation they may provide upon use of the product comprising the composition. In some embodiments, the organic acid is monomenthyl succinate, monomenthyl fumarate, monomenthyl glutarate, or a combination thereof. In some embodiments, the organic acid is a monotocopheryl ester of a dicarboxylic acid. Certain tocopheryl esters may be desirable in oral compositions as described herein by virtue of the antioxidant effects they may provide. In some embodiments, the organic acid is tocopheryl succinate, tocopheryl fumarate, tocopheryl glutarate, or a combination thereof.

In some embodiments, the organic acid is a carotenoid derivative having one or more carboxylic acids. Carotenoids are tetraterpenes, meaning that they are produced from 8 isoprene molecules and contain 40 carbon atoms. Accordingly, they are usually lipophilic due to the presence of long unsaturated aliphatic chains, and are generally yellow, orange, or red in color. Certain carotenoid derivatives can be advantageous in oral compositions by virtue of providing both ion pairing and serving as a colorant in the composition. In some embodiments, the organic acid is 2E,4E,6E,8E,10E,12E,14E,16Z,18E)-20-methoxy-4, 8,13,17-tetramethyl-20-oxoicosa-4,6,8,10,12,14,16,18-nonaenoic acid (bixin) or an isomer thereof. Bixin is an apocarotenoid found in annatto seeds from the achiote tree (Bixa orellana), and is the naturally occurring pigment providing the reddish orange color to annatto. Bixin is soluble in fats and alcohols but insoluble in water, and is chemically unstable when isolated, converting via isomerization into the double bond isomer, trans-bixin (β-bixin), having the structure:

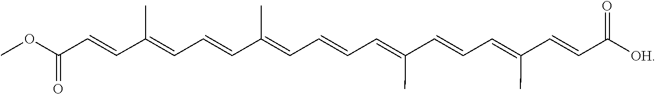 [SMILES: COC(=O)/C=C/C(C)=C/C=C/C(C)=C/C=C/C=C(C)/C=C/C=C(C)/C=C/C(=O)O]

In some embodiments, the organic acid is (2E4E,6E,8E,10E, 12E, 14E,16E,18E)-4,8,13,17-tetramethylicosa-2,4,6,8,10,12,14,16,18-nonaenedioic acid (norbixin), a water soluble hydrolysis product of bixin having the structure:

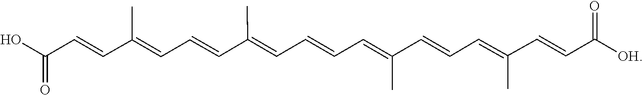 [SMILES: CC(/C=C/C=C(C)/C=C/C(=O)O)=C\C=C\C=C(C)\C=C\C=C(C)\C=C\C(=O)O]

The selection of organic acid may further depend on additional properties in addition to or without consideration to the logP value. For example, an organic acid should be one recognized as safe for human consumption, and which has acceptable flavor, odor, volatility, stability, and the like. Determination of appropriate organic acids is within the purview of one of skill in the art.

In some embodiments, the organic acid is benzoic acid, a toluic acid, benzenesulfonic acid, toluenesulfonic acid, hexanoic acid, heptanoic acid, decanoic acid, or octanoic acid. In some embodiments, the organic acid is benzoic acid, octanoic acid, or decanoic acid. In some embodiments, the organic acid is octanoic acid.

In some embodiments, more than one organic acid may be present. For example, the composition may comprise two, or three, or four, or more organic acids. Accordingly, reference herein to “an organic acid” contemplates mixtures of two or more organic acids. The relative amounts of the multiple organic acids may vary. For example, a composition may comprise equal amounts of two, or three, or more organic acids, or may comprise different relative amounts. In this manner, it is possible to include certain organic acids (e.g., citric acid or myristic acid) which have a logP value outside the desired range, when combined with other organic acids to provide the desired average logP range for the combination. In some embodiments, it may be desirable to include organic acids in the composition which have logP values outside the desired range for purposes such as, but not limited to, providing desirable organoleptic properties, stability, as flavor components, and the like. Further, certain lipophilic organic acids have undesirable flavor and or aroma characteristics which would preclude their presence as the sole organic acid (e.g., in equimolar or greater quantities relative to nicotine). Without wishing to be bound by theory, it is believed that a combination of different organic acids may provide the desired ion pairing while the concentration of any single organic acid in the composition remains below the threshold which would be found objectionable from a sensory perspective.

For example, in some embodiments, the organic acid may comprise from about 1 to about 5 or more molar equivalents of benzoic acid relative to nicotine, combined with e.g., about 0.2 molar equivalents of octanoic acid or a salt thereof, and 0.2 molar equivalents of decanoic acid or a salt thereof.

In some embodiments, the organic acid is a combination of any two organic acids selected from the group consisting of benzoic acid, a toluic acid, benzenesulfonic acid, toluenesulfonic acid, hexanoic acid, heptanoic acid, decanoic acid, and octanoic acid. In some embodiments, the organic acid is a combination of benzoic acid, octanoic acid, and decanoic acid, or benzoic and octanoic acid. In some embodiments, the composition comprises citric acid in addition to one or more of benzoic acid, a toluic acid, benzenesulfonic acid, toluenesulfonic acid, hexanoic acid, heptanoic acid, decanoic acid, and octanoic acid.

In some embodiments, the composition comprises an alkali metal salt of an organic acid. For example, at least a portion of the organic acid may be present in the composition in the form of an alkali metal salt. Suitable alkali metal salts include lithium, sodium, and potassium. In some embodiments, the alkali metal is sodium or potassium. In some embodiments, the alkali metal is sodium. In some embodiments, the composition comprises an organic acid and a sodium salt of the organic acid.

In some embodiments, the composition comprises benzoic acid and sodium benzoate, octanoic acid and sodium octanoate, decanoic acid and sodium decanoate, or a combination thereof.

In some embodiments, the ratio of the organic acid to the sodium salt of the organic acid is from about 0.1 to about 10, such as from about 0.1, about 0.25, about 0.3, about 0.5, about 0.75, or about 1, to about 2, about 5, or about 10. For example, in some embodiments, both an organic acid and the sodium salt thereof are added to the other components of the composition, wherein the organic acid is added in excess of the sodium salt, in equimolar quantities with the sodium salt, or as a fraction of the sodium salt. One of skill in the art will recognize that the relative amounts will be determined by the desired pH of the composition, as well as the desired ionic strength. For example, the organic acid may be added in a quantity to provide a desired pH level of the composition, while the alkali metal (e.g., sodium) salt is added in a quantity to provide the desired extent of ion pairing. As one of skill in the art will understand, the quantity of organic acid (i.e., the protonated form) present in the composition, relative to the alkali metal salt or conjugate base form present in the composition, will vary according to the pH of the composition and the pKa of the organic acid, as well as according to the actual relative quantities initially added to the composition.

The amount of organic acid or an alkali metal salt thereof present in the composition, relative to nicotine, may vary. Generally, as the concentration of the organic acid (or the conjugate base thereof) increases, the percent of nicotine that is ion paired with the organic acid increases. This typically increases the partitioning of the nicotine, in the form of an ion pair, into octanol versus water as measured by the logP (the log₁₀ of the partitioning coefficient). In some embodiments, the composition comprises from about 0.05, about 0.1, about 1, about 1.5, about 2, or about 5, to about 10, about 15, or about 20 molar equivalents of the organic acid, the alkali metal salt thereof, or the combination thereof, relative to the nicotine component, calculated as free base nicotine.

In some embodiments, the composition comprises from about 2 to about 10, or from about 2 to about 5 molar equivalents of the organic acid, the alkali metal salt thereof, or the combination thereof, to nicotine, on a free-base nicotine basis. In some embodiments, the organic acid, the alkali metal salt thereof, or the combination thereof, is present in a molar ratio with the nicotine from about 2, about 3, about 4, or about 5, to about 6, about 7, about 8, about 9, or about 10. In embodiments wherein more than one organic acid, alkali metal salt thereof, or both, are present, it is to be understood that such molar ratios reflect the totality of the organic acids present.

In certain embodiments the organic acid inclusion is sufficient to provide a composition pH of from about 4.0 to about 9.0, such as from about 4.5 to about 7.0, or from about 5.5 to about 7.0, from about 4.0 to about 5.5, or from about 7.0 to about 9.0. In some embodiments, the organic acid inclusion is sufficient to provide a composition pH of from about 4.5 to about 6.5, for example, from about 4.5, about 5.0, or about 5.5, to about 6.0, or about 6.5. In some embodiments, the organic acid is provided in a quantity sufficient to provide a pH of the composition of from about 5.5 to about 6.5, for example, from about 5.5, about 5.6, about 5.7, about 5.8, about 5.9, or about 6.0, to about 6.1, about 6.2, about 6.3, about 6.4, or about 6.5. In other embodiments, a mineral acid (e.g., hydrochloric acid, sulfuric acid, phosphoric acid, or the like) is added to adjust the pH of the composition to the desired value.

In some embodiments, the organic acid is added as the free acid, either neat (i.e., native solid or liquid form) or as a solution in, e.g., water, to the other composition components. In some embodiments, the alkali metal salt of the organic acid is added, either neat or as a solution in, e.g., water, to the other composition components. In some embodiments, the organic acid and the basic amine (e.g., nicotine) are combined to form a salt, either before addition to the composition, or the salt is formed within and is present in the composition as such. In other embodiments, the organic acid and basic amine (e.g., nicotine) are present as individual components in the composition, and form an ion pair upon contact with moisture (e.g., saliva in the mouth of the consumer).

In some embodiments, the composition comprises nicotine benzoate and sodium benzoate (or other alkali metal benzoate). In other embodiments, the composition comprises nicotine and an organic acid, wherein the organic acid is a monoester of a dicarboxylic acid or is a carotenoid derivative having one or more carboxylic acids.

In some embodiments, at least a portion of the nicotine can be in the form of a resin complex of nicotine, where nicotine is bound in an ion-exchange resin, such as nicotine polacrilex, which is nicotine bound to, for example, a polymethacrylic acid, such as Amberlite IRP64, Purolite C115HMR, or Doshion P551. See, for example, U.S. Pat. No. 3,901,248 to Lichtneckert et al., which is incorporated herein by reference. Another example is a nicotine-polyacrylic carbomer complex, such as with Carbopol 974P. In some embodiments, nicotine may be present in the form of a nicotine polyacrylic complex.

Typically, the nicotine component (calculated as the free base) when present, is in a concentration of at least about 0.001% by weight of the mixture, such as in a range from about 0.001% to about 10%. In some embodiments, the nicotine component is present in a concentration from about 0.1% w/w to about 10% by weight, such as, e.g., from about 0.1% w/w, about 0.2%, about 0.3%, about 0.4%, about 0.5% about 0.6%, about 0.7%, about 0.8%, or about 0.9%, to about 1%, about 2%, about 3%, about 4%, about 5%, about 6%, about 7%, about 8%, about 9%, or about 10% by weight, calculated as the free base and based on the total weight of the mixture. In some embodiments, the nicotine component is present in a concentration from about 0.1% w/w to about 3% by weight, such as, e.g., from about 0.1% w/w to about 2.5%, from about 0.1% to about 2.0%, from about 0.1% to about 1.5%, or from about 0.1% to about 1% by weight, calculated as the free base and based on the total weight of the edible, orally dissolvable film. These ranges can also apply to other active ingredients noted herein.

In some embodiments, the products or compositions of the disclosure can be characterized as free of any nicotine component (e.g., any embodiment as disclosed herein may be completely or substantially free of any nicotine component). By “substantially free” is meant that no nicotine has been intentionally added, beyond trace amounts that may be naturally present in e.g., a botanical material. For example, certain embodiments can be characterized as having less than 0.001% by weight of nicotine, or less than 0.0001%, or even 0% by weight of nicotine, calculated as the free base.

In some embodiments, the active ingredient comprises a nicotine component (e.g., any product or composition of the disclosure, in addition to comprising any active ingredient or combination of active ingredients as disclosed herein, may further comprise a nicotine component).

### Cannabinoids

In some embodiments, the active ingredient comprises one or more cannabinoids. As used herein, the term “cannabinoid” refers to a class of diverse natural or synthetic chemical compounds that acts on cannabinoid receptors (i.e., CB1 and CB2) in cells that alter neurotransmitter release in the brain. Cannabinoids are cyclic molecules exhibiting particular properties such as the ability to easily cross the blood-brain barrier. Cannabinoids may be naturally occurring (Phytocannabinoids) from plants such as cannabis, (endocannabinoids) from animals, or artificially manufactured (synthetic cannabinoids). Cannabis species express at least 85 different phytocannabinoids, and these may be divided into subclasses, including cannabigerols, cannabichromenes, cannabidiols, tetrahydrocannabinols, cannabinols and cannabinodiols, and other cannabinoids, such as cannabigerol (CBG), cannabichromene (CBC), cannabidiol (CBD), tetrahydrocannabinol (THC), cannabinol (CBN) and cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), Cannabinol propyl variant (CBNV), cannabitriol (CBO), tetrahydrocannabmolic acid (THCA), and tetrahydrocannabivarinic acid (THCV A).

In some embodiments, the cannabinoid is selected from the group consisting of cannabigerol (CBG), cannabichromene (CBC), cannabidiol (CBD), tetrahydrocannabinol (THC), cannabinol (CBN) and cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), Cannabinol propyl variant (CBNV), cannabitriol (CBO), tetrahydrocannabmolic acid (THCA), tetrahydrocannabivarinic acid (THCV A), and mixtures thereof. In some embodiments, the cannabinoid comprises at least tetrahydrocannabinol (THC). In some embodiments, the cannabinoid is tetrahydrocannabinol (THC). In some embodiments, the cannabinoid comprises at least cannabidiol (CBD). In some embodiments, the cannabinoid is cannabidiol (CBD). In some embodiments, the CBD is synthetic CBD. Notably, CBD has a logP value of about 6.5, making it insoluble in an aqueous environment (e.g., saliva).

In some embodiments, the cannabinoid (e.g., CBD) is added to the oral product in the form of an isolate. An isolate is an extract from a plant, such as cannabis, where the active material of interest (in this case the cannabinoid, such as CBD) is present in a high degree of purity, for example greater than 95%, greater than 96%, greater than 97%, greater than 98%, or around 99% purity.

In some embodiments, the cannabinoid is an isolate of CBD in a high degree of purity, and the amount of any other cannabinoid in the oral product is no greater than about 1% by weight of the oral product, such as no greater than about 0.5% by weight of the oral product, such as no greater than about 0.1% by weight of the oral product, such as no greater than about 0.01% by weight of the oral product.

The choice of cannabinoid and the particular percentages thereof which may be present within the disclosed oral product will vary depending upon the desired flavor, texture, and other characteristics of the oral product.

Alternatively, or in addition to the cannabinoid, the active agent may include a cannabimimetic, which is a class of compounds derived from plants other than cannabis that have biological effects on the endocannabinoid system similar to cannabinoids. Examples include yangonin, alpha-amyrin or beta-amyrin (also classified as terpenes), cyanidin, curcumin (tumeric), catechin, quercetin, salvinorin A, N-acylethanolamines, and N-alkylamide lipids. Such compounds can be used in the same amounts and ratios noted herein for cannabinoids.

When present, a cannabinoid (e.g., CBD) or cannabimimetic is typically in a concentration of at least about 0.1% by weight of the composition, such as in a range from about 0.1% to about 30%, such as, e.g., from about 0.1%, about 0.2%, about 0.3%, about 0.4%, about 0.5% about 0.6%, about 0.7%, about 0.8%, or about 0.9%, to about 1%, about 2%, about 3%, about 4%, about 5%, about 6%, about 7%, about 8%, about 9%, about 10%, about 15%, about 20%, or about 30% by weight, based on the total weight of the composition. In some embodiments, the cannabinoid (such as CBD) is present in the oral product in a concentration of at least about 0.001% by weight of the oral product, such as in a range from about 0.001% to about 2% by weight of the oral product. In some embodiments, the cannabinoid (such as CBD) is present in the oral product in a concentration of from about 0.1% to about 1.5% by weight, based on the total weight of the oral product. In some embodiments, the cannabinoid (such as CBD) is present in a concentration from about 0.4% to about 1.5% by weight, based on the total weight of the oral product.

### Terpenes

Active ingredients suitable for use in the present disclosure can also be classified as terpenes, many of which are associated with biological effects, such as calming effects. Terpenes are understood to have the general formula of (C₅H₈)ₙ and include monoterpenes, sesquiterpenes, and diterpenes. Terpenes can be acyclic, monocyclic or bicyclic in structure. Some terpenes provide an entourage effect when used in combination with cannabinoids or cannabimimetics. Examples include beta-caryophyllene, linalool, limonene, beta-citronellol, linalyl acetate, pinene (alpha or beta), geraniol, carvone, eucalyptol, menthone, iso-menthone, piperitone, myrcene, beta-bourbonene, and germacrene, which may be used singly or in combination.

In some embodiments, the terpene is a terpene derivable from a phytocannabinoid producing plant, such as a plant from the stain of the cannabis sativa species, such as hemp. Suitable terpenes in this regard include so-called “C10” terpenes, which are those terpenes comprising 10 carbon atoms, and so-called “C15” terpenes, which are those terpenes comprising 15 carbon atoms. In some embodiments, the active ingredient comprises more than one terpene. For example, the active ingredient may comprise one, two, three, four, five, six, seven, eight, nine, ten or more terpenes as defined herein. In some embodiments, the terpene is selected from pinene (alpha and beta), geraniol, linalool, limonene, carvone, eucalyptol, menthone, iso-menthone, piperitone, myrcene, beta-bourbonene, germacrene and mixtures thereof.

### Pharmaceutical Ingredients

In some embodiments, the active ingredient comprises an active pharmaceutical ingredient (API). The API can be any known agent adapted for therapeutic, prophylactic, or diagnostic use. These can include, for example, synthetic organic compounds, proteins and peptides, polysaccharides and other sugars, lipids, phospholipids, inorganic compounds (e.g., magnesium, selenium, zinc, nitrate), neurotransmitters or precursors thereof (e.g., serotonin, 5-hydroxytryptophan, oxitriptan, acetylcholine, dopamine, melatonin), and nucleic acid sequences, having therapeutic, prophylactic, or diagnostic activity. Non-limiting examples of APIs include analgesics and antipyretics (e.g., acetylsalicylic acid, acetaminophen, 3-(4-isobutylphenyl)propanoic acid), phosphatidylserine, myoinositol, docosahexaenoic acid (DHA, Omega-3), arachidonic acid (AA, Omega-6), S-adenosylmethionine (SAM), beta-hydroxy-beta-methylbutyrate (HMB), citicoline (cytidine-5′-diphosphate-choline), and cotinine. In some embodiments, the active ingredient comprises citicoline. In some embodiments, the active ingredient is a combination of citicoline, caffeine, theanine, and ginseng. In some embodiments, the active ingredient comprises sunflower lecithin. In some embodiments, the active ingredient is a combination of sunflower lecithin, caffeine, theanine, and ginseng.

The amount of API may vary. For example, when present, an API is typically at a concentration of from about 0.001% w/w to about 10% by weight, such as, e.g., from about 0.01%, about 0.02%, about 0.03%, about 0.04%, about 0.05%, about 0.06%, about 0.07%, about 0.08%, about 0.09%, about 0.1% w/w, about 0.2%, about 0.3%, about 0.4%, about 0.5% about 0.6%, about 0.7%, about 0.8%, about 0.9%, or about 1%, to about 2%, about 3%, about 4%, about 5%, about 6%, about 7%, about 8%, about 9%, or about 10% by weight, based on the total weight of the composition.

In some embodiments, the edible, orally dissolvable film or product comprising such film is substantially free of any API. By “substantially free of any API” means that the composition does not contain, and specifically excludes, the presence of any API as defined herein, such as any Food and Drug Administration (FDA) approved therapeutic agent intended to treat any medical condition.

### Tobacco Material

In some embodiments, the edible, orally dissolvable film may include a tobacco material. The tobacco material can vary in species, type, and form. Generally, the tobacco material is obtained from for a harvested plant of the Nicotiana species. Example Nicotiana species include N. tabacum, N. rustica, N. alata, N. arentsii, N. excelsior, N. forgetiana, N. glauca, N. glutinosa, N. gossei, N. kawakamii, N. knightiana, N. langsdorffi, N. otophora, N. setchelli, N. sylvestris, N. tomentosa, N. tomentosiformis, N. undulata, N. x sanderae, N. africana, N. amplexicaulis, N. benavidesii, N. bonariensis, N. debneyi, N. longiflora, N. maritina, N. megalosiphon, N. occidentalis, N. paniculata, N. plumbaginifolia, N. raimondii, N. rosulata, N. simulans, N. stocktonii, N. suaveolens, N. umbratica, N. velutina, N. wigandioides, N. acaulis, N. acuminata, N. attenuata, N. benthamiana, N. cavicola, N. clevelandii, N. cordifolia, N. corymbosa, N. fragrans, N. goodspeedii, N. linearis, N. miersii, N. nudicaulis, N. obtusifolia, N. occidentalis subsp. Hersperis, N. pauciflora, N. petunioides, N. quadrivalvis, N. repanda, N. rotundifolia, N. solanifolia, and N. spegazzinii. Various representative other types of plants from the Nicotiana species are set forth in Goodspeed, The Genus Nicotiana, (Chonica Botanica) (1954); U.S. Pat. No. 4,660,577 to Sensabaugh, Jr. et al.; U.S. Pat. No. 5,387,416 to White et al., U.S. Pat. No. 7,025,066 to Lawson et al.; U.S. Pat. No. 7,798,153 to Lawrence, Jr. and U.S. Pat. No. 8,186,360 to Marshall et al.; each of which is incorporated herein by reference. Descriptions of various types of tobaccos, growing practices and harvesting practices are set forth in Tobacco Production, Chemistry and Technology, Davis et al. (Eds.) (1999), which is incorporated herein by reference.

Nicotiana species from which suitable tobacco materials can be obtained can be derived using genetic-modification or crossbreeding techniques (e.g., tobacco plants can be genetically engineered or crossbred to increase or decrease production of components, characteristics or attributes). See, for example, the types of genetic modifications of plants set forth in U.S. Pat. No. 5,539,093 to Fitzmaurice et al.; U.S. Pat. No. 5,668,295 to Wahab et al.; U.S. Pat. No. 5,705,624 to Fitzmaurice et al.; U.S. Pat. No. 5,844,119 to Weigl; U.S. Pat. No. 6,730,832 to Dominguez et al.; U.S. Pat. No. 7,173,170 to Liu et al.; U.S. Pat. No. 7,208,659 to Colliver et al. and U.S. Pat. No. 7,230,160 to Benning et al.; US Patent Appl. Pub. No. 2006/0236434 to Conkling et al.; and PCT WO2008/103935 to Nielsen et al. See, also, the types of tobaccos that are set forth in U.S. Pat. No. 4,660,577 to Sensabaugh, Jr. et al.; 5,387,416 to White et al.; and U.S. Pat. No. 6,730,832 to Dominguez et al., each of which is incorporated herein by reference.

The Nicotiana species can, in some embodiments, be selected for the content of various compounds that are present therein. For example, plants can be selected on the basis that those plants produce relatively high quantities of one or more of the compounds desired to be isolated therefrom. In certain embodiments, plants of the Nicotiana species (e.g., Galpao commun tobacco) are specifically grown for their abundance of leaf surface compounds. Tobacco plants can be grown in greenhouses, growth chambers, or outdoors in fields, or grown hydroponically.

Various parts or portions of the plant of the Nicotiana species can be included within a mixture as disclosed herein. For example, virtually all of the plant (e.g., the whole plant) can be harvested, and employed as such. Alternatively, various parts or pieces of the plant can be harvested or separated for further use after harvest. For example, the flower, leaves, stem, stalk, roots, seeds, and various combinations thereof, can be isolated for further use or treatment. In some embodiments, the tobacco material comprises tobacco leaf (lamina). The mixture disclosed herein can include processed tobacco parts or pieces, cured and aged tobacco in essentially natural lamina and/or stem form, a tobacco extract, extracted tobacco pulp (e.g., using water as a solvent), or a mixture of the foregoing (e.g., a mixture that combines extracted tobacco pulp with granulated cured and aged natural tobacco lamina).

In certain embodiments, the tobacco material comprises solid tobacco material selected from the group consisting of lamina and stems. The tobacco that is used for the mixture most preferably includes tobacco lamina, or a tobacco lamina and stem mixture (of which at least a portion is smoke-treated). Portions of the tobaccos within the mixture may have processed forms, such as processed tobacco stems (e.g., cut-rolled stems, cut-rolled-expanded stems or cut-puffed stems), or volume expanded tobacco (e.g., puffed tobacco, such as dry ice expanded tobacco (DIET)). See, for example, the tobacco expansion processes set forth in U.S. Pat. No. 4,340,073 to de la Burde et al.; U.S. Pat. No. 5,259,403 to Guy et al.; and U.S. Pat. No. 5,908,032 to Poindexter, et al.; and U.S. Pat. No. 7,556,047 to Poindexter, et al., all of which are incorporated by reference. In addition, the d mixture optionally may incorporate tobacco that has been fermented. See, also, the types of tobacco processing techniques set forth in PCT WO2005/063060 to Atchley et al., which is incorporated herein by reference.

The tobacco material is typically used in a form that can be described as particulate (i.e., shredded, ground, granulated, or powder form). The manner by which the tobacco material is provided in a finely divided or powder type of form may vary. Preferably, plant parts or pieces are comminuted, ground or pulverized into a particulate form using equipment and techniques for grinding, milling, or the like. Most preferably, the plant material is relatively dry in form during grinding or milling, using equipment such as hammer mills, cutter heads, air control mills, or the like. For example, tobacco parts or pieces may be ground or milled when the moisture content thereof is less than about 15 weight percent or less than about 5 weight percent. Most preferably, the tobacco material is employed in the form of parts or pieces that have an average particle size between 1.4 millimeters and 250 microns. In some instances, the tobacco particles may be sized to pass through a screen mesh to obtain the particle size range required. If desired, air classification equipment may be used to ensure that small sized tobacco particles of the desired sizes, or range of sizes, may be collected. If desired, differently sized pieces of granulated tobacco may be mixed together.

The manner by which the tobacco is provided in a finely divided or powder type of form may vary. Preferably, tobacco parts or pieces are comminuted, ground or pulverized into a powder type of form using equipment and techniques for grinding, milling, or the like. Most preferably, the tobacco is relatively dry in form during grinding or milling, using equipment such as hammer mills, cutter heads, air control mills, or the like. For example, tobacco parts or pieces may be ground or milled when the moisture content thereof is less than about 15 weight percent to less than about 5 weight percent. For example, the tobacco plant or portion thereof can be separated into individual parts or pieces (e.g., the leaves can be removed from the stems, and/or the stems and leaves can be removed from the stalk). The harvested plant or individual parts or pieces can be further subdivided into parts or pieces (e.g., the leaves can be shredded, cut, comminuted, pulverized, milled or ground into pieces or parts that can be characterized as filler-type pieces, granules, particulates or fine powders). The plant, or parts thereof, can be subjected to external forces or pressure (e.g., by being pressed or subjected to roll treatment). When carrying out such processing conditions, the plant or portion thereof can have a moisture content that approximates its natural moisture content (e.g., its moisture content immediately upon harvest), a moisture content achieved by adding moisture to the plant or portion thereof, or a moisture content that results from the drying of the plant or portion thereof. For example, powdered, pulverized, ground or milled pieces of plants or portions thereof can have moisture contents of less than about 25 weight percent, often less than about 20 weight percent, and frequently less than about 15 weight percent.

For the preparation of oral products, it is typical for a harvested plant of the Nicotiana species to be subjected to a curing process. The tobacco materials incorporated within the mixture for inclusion within products as disclosed herein are those that have been appropriately cured and/or aged. Descriptions of various types of curing processes for various types of tobaccos are set forth in Tobacco Production, Chemistry and Technology, Davis et al. (Eds.) (1999). Examples of techniques and conditions for curing flue-cured tobacco are set forth in Nestor et al., Beitrage Tabakforsch. Int., 20, 467-475 (2003) and U.S. Pat. No. 6,895,974 to Peele, which are incorporated herein by reference. Representative techniques and conditions for air curing tobacco are set forth in U.S. Pat. No. 7,650,892 to Groves et al.; Roton et al., Beitrage Tabakforsch. Int., 21, 305-320 (2005) and Staaf et al., Beitrage Tabakforsch. Int., 21, 321-330 (2005), which are incorporated herein by reference. Certain types of tobaccos can be subjected to alternative types of curing processes, such as fire curing or sun curing.

In certain embodiments, tobacco materials that can be employed include flue-cured or Virginia (e.g., K326), burley, sun-cured (e.g., Indian Kurnool and Oriental tobaccos, including Katerini, Prelip, Komotini, Xanthi and Yambol tobaccos), Maryland, dark, dark-fired, dark air cured (e.g., Madole, Passanda, Cubano, Jatin and Bezuki tobaccos), light air cured (e.g., North Wisconsin and Galpao tobaccos), Indian air cured, Red Russian and Rustica tobaccos, as well as various other rare or specialty tobaccos and various blends of any of the foregoing tobaccos.

The tobacco material may also have a so-called “blended” form. For example, the tobacco material may include a mixture of parts or pieces of flue-cured, burley (e.g., Malawi burley tobacco) and Oriental tobaccos (e.g., as tobacco composed of, or derived from, tobacco lamina, or a mixture of tobacco lamina and tobacco stem). For example, a representative blend may incorporate about 30 to about 70 parts burley tobacco (e.g., lamina, or lamina and stem), and about 30 to about 70 parts flue cured tobacco (e.g., stem, lamina, or lamina and stem) on a dry weight basis. Other example tobacco blends incorporate about 75 parts flue-cured tobacco, about 15 parts burley tobacco, and about 10 parts Oriental tobacco; or about 65 parts flue-cured tobacco, about 25 parts burley tobacco, and about 10 parts Oriental tobacco; or about 65 parts flue-cured tobacco, about 10 parts burley tobacco, and about 25 parts Oriental tobacco; on a dry weight basis. Other example tobacco blends incorporate about 20 to about 30 parts Oriental tobacco and about 70 to about 80 parts flue-cured tobacco on a dry weight basis.

Tobacco materials used in the present disclosure can be subjected to, for example, fermentation, bleaching, and the like. If desired, the tobacco materials can be, for example, irradiated, pasteurized, or otherwise subjected to controlled heat treatment. Such treatment processes are detailed, for example, in U.S. Pat. No. 8,061,362 to Mua et al., which is incorporated herein by reference. In certain embodiments, tobacco materials can be treated with water and an additive capable of inhibiting reaction of asparagine to form acrylamide upon heating of the tobacco material (e.g., an additive selected from the group consisting of lysine, glycine, histidine, alanine, methionine, cysteine, glutamic acid, aspartic acid, proline, phenylalanine, valine, arginine, compositions incorporating di- and trivalent cations, asparaginase, certain non-reducing saccharides, certain reducing agents, phenolic compounds, certain compounds having at least one free thiol group or functionality, oxidizing agents, oxidation catalysts, natural plant extracts (e.g., rosemary extract), and combinations thereof. See, for example, the types of treatment processes described in U.S. Pat. Nos. 8,434,496, 8,944,072, and 8,991,403 to Chen et al., which are all incorporated herein by reference. In certain embodiments, this type of treatment is useful where the original tobacco material is subjected to heat in the processes previously described.

In some embodiments, the type of tobacco material is selected such that it is initially visually lighter in color than other tobacco materials to some degree (e.g., whitened or bleached). Tobacco pulp can be whitened in certain embodiments according to any means known in the art. For example, bleached tobacco material produced by various whitening methods using various bleaching or oxidizing agents and oxidation catalysts can be used. Example oxidizing agents include peroxides (e.g., hydrogen peroxide), chlorite salts, chlorate salts, perchlorate salts, hypochlorite salts, ozone, ammonia, potassium permanganate, and combinations thereof. Example oxidation catalysts are titanium dioxide, manganese dioxide, and combinations thereof. Processes for treating tobacco with bleaching agents are discussed, for example, in U.S. Pat. Nos. 787,611 to Daniels, Jr.; 1,086,306 to Oelenheinz; 1,437,095 to Delling; 1,757,477 to Rosenhoch; 2,122,421 to Hawkinson; 2,148,147 to Baier; 2,170,107 to Baier; 2,274,649 to Baier; 2,770,239 to Prats et al.; 3,612,065 to Rosen; 3,851,653 to Rosen; 3,889,689 to Rosen; 3,943,940 to Minami; 3,943,945 to Rosen; 4,143,666 to Rainer; 4,194,514 to Campbell; 4,366,823, 4,366,824, and 4,388,933 to Rainer et al.; 4,641,667 to Schmekel et al.; 5,713,376 to Berger; 9,339,058 to Byrd Jr. et al.; 9,420,825 to Beeson et al.; and 9,950,858 to Byrd Jr. et al.; as well as in US Pat. App. Pub. Nos. 2012/0067361 to Bjorkholm et al.; 2016/0073686 to Crooks; 2017/0020183 to Bjorkholm; and 2017/0112183 to Bjorkholm, and in PCT Publ. Appl. Nos. WO1996/031255 to Giolvas and WO2018/083114 to Bjorkholm, all of which are incorporated herein by reference.

In some embodiments, the whitened tobacco material can have an ISO brightness of at least about 50%, at least about 60%, at least about 65%, at least about 70%, at least about 75%, or at least about 80%. In some embodiments, the whitened tobacco material can have an ISO brightness in the range of about 50% to about 90%, about 55% to about 75%, or about 60% to about 70%. ISO brightness can be measured according to ISO 3688:1999 or ISO 2470-1:2016.

In some embodiments, the whitened tobacco material can be characterized as lightened in color (e.g., “whitened”) in comparison to an untreated tobacco material. White colors are often defined with reference to the International Commission on Illumination's (CIE's) chromaticity diagram. The whitened tobacco material can, in certain embodiments, be characterized as closer on the chromaticity diagram to pure white than an untreated tobacco material.

In various embodiments, the tobacco material can be treated to extract a soluble component of the tobacco material therefrom. “Tobacco extract” as used herein refers to the isolated components of a tobacco material that are extracted from solid tobacco pulp by a solvent that is brought into contact with the tobacco material in an extraction process. Various extraction techniques of tobacco materials can be used to provide a tobacco extract and tobacco solid material. See, for example, the extraction processes described in US Pat. Appl. Pub. No. 2011/0247640 to Beeson et al., which is incorporated herein by reference. Other example techniques for extracting components of tobacco are described in U.S. Pat. No. 4,144,895 to Fiore; U.S. Pat. No. 4,150,677 to Osborne, Jr. et al.; U.S. Pat. No. 4,267,847 to Reid; U.S. Pat. No. 4,289,147 to Wildman et al.; U.S. Pat. No. 4,351,346 to Brummer et al.; U.S. Pat. No. 4,359,059 to Brummer et al.; U.S. Pat. No. 4,506,682 to Muller; U.S. Pat. No. 4,589,428 to Keritsis; U.S. Pat. No. 4,605,016 to Soga et al.; U.S. Pat. No. 4,716,911 to Poulose et al.; U.S. Pat. No. 4,727,889 to Niven, Jr. et al.; U.S. Pat. No. 4,887,618 to Bernasek et al.; U.S. Pat. No. 4,941,484 to Clapp et al.; U.S. Pat. No. 4,967,771 to Fagg et al.; U.S. Pat. No. 4,986,286 to Roberts et al.; U.S. Pat. No. 5,005,593 to Fagg et al.; U.S. Pat. No. 5,018,540 to Grubbs et al.; U.S. Pat. No. 5,060,669 to White et al.; U.S. Pat. No. 5,065,775 to Fagg; U.S. Pat. No. 5,074,319 to White et al.; U.S. Pat. No. 5,099,862 to White et al.; U.S. Pat. No. 5,121,757 to White et al.; U.S. Pat. No. 5,131,414 to Fagg; U.S. Pat. No. 5,131,415 to Munoz et al.; U.S. Pat. No. 5,148,819 to Fagg; U.S. Pat. No. 5,197,494 to Kramer; U.S. Pat. No. 5,230,354 to Smith et al.; U.S. Pat. No. 5,234,008 to Fagg; U.S. Pat. No. 5,243,999 to Smith; U.S. Pat. No. 5,301,694 to Raymond et al.; U.S. Pat. No. 5,318,050 to Gonzalez-Parra et al.; U.S. Pat. No. 5,343,879 to Teague; U.S. Pat. No. 5,360,022 to Newton; U.S. Pat. No. 5,435,325 to Clapp et al.; U.S. Pat. No. 5,445,169 to Brinkley et al.; U.S. Pat. No. 6,131,584 to Lauterbach; U.S. Pat. No. 6,298,859 to Kierulff et al.; U.S. Pat. No. 6,772,767 to Mua et al.; and U.S. Pat. No. 7,337,782 to Thompson, all of which are incorporated by reference herein.

Typical inclusion ranges for tobacco materials can vary depending on the nature and type of the tobacco material, and the intended effect on the final edible, orally dissolvable film or product comprising such film, with an example range of up to about 30% by weight (or up to about 20% by weight or up to about 10% by weight or up to about 5% by weight), based on total weight of the edible, orally dissolvable film or product comprising such film (e.g., about 0.1 to about 15% by weight).

In some embodiments, the edible, orally dissolvable film or product comprising such film of the disclosure can be characterized as completely free or substantially free of tobacco material (other than purified nicotine as an active ingredient, in some embodiments). For example, certain embodiments can be characterized as having less than 1% by weight, or less than 0.5% by weight, or less than 0.1% by weight of tobacco material, or 0% by weight of tobacco material.

### Other Components

In addition to the binder component, the plasticizer component, and the optional active ingredient, the edible, orally dissolvable films provided herein can comprise one or more of a number of additional components. For example, they can comprise components such as fillers, flavorants, colorants, salts, sweeteners, preservatives, permeation enhancers, emulsifiers, surfactants, buffering agents/pH adjusters, processing aids, and the like.

Fillers (which can also, in some embodiments, be referred to as “bulking agents”) can, in some embodiments, be components that are similar in chemical structure to the components referenced herein above with respect to binders; however, fillers have low film-forming characteristics. In some embodiments, a filler may function to alter the dissolution character of the film structure. The fillers can be particulate or soluble in the mixture of components used to prepare an edible, orally dissolvable film as provided herein. The fillers employed in the disclosed films can comprise a single type of filler or more than one (e.g., two or more or three or more) different fillers.

Examples of suitable fillers include, but are not limited to, carbohydrates, cellulose, fiber, starch, maltodextrin, polyglycitols, polysaccharides (non-film-forming polysaccharides), and minerals. For example, suitable fillers can be any non-tobacco plant material or derivative thereof, including cellulose materials derived from such sources. Examples of cellulosic non-tobacco plant material include cereal grains (e.g., maize, oat, barley, rye, buckwheat, and the like), sugar beet (e.g., FIBREX® brand filler available from International Fiber Corporation), bran fiber, and mixtures thereof. Non-limiting examples of derivatives of non-tobacco plant material include starches (e.g., from potato, wheat, rice, corn), natural cellulose, and modified cellulosic materials. In some embodiments, fillers comprise a mixture of glucose and starch-derived polysaccharides. One such suitable mixture of glucose and starch-derived polysaccharides is EMDEX , available from JRS PHARMA LP, USA, 2981 Route 22, Patterson, N.Y. 12563-2359. Additional examples of potential filler components include maltodextrin, dextrose, calcium carbonate, calcium phosphate, lactose, mannitol, xylitol, and sorbitol. Combinations of fillers can also be used.

“Starch” as used herein may refer to pure starch from any source, modified starch, or starch derivatives. Starch is present, typically in granular form, in almost all green plants and in various types of plant tissues and organs (e.g., seeds, leaves, rhizomes, roots, tubers, shoots, fruits, grains, and stems). Starch can vary in composition, as well as in granular shape and size. Often, starch from different sources has different chemical and physical characteristics. A specific starch can be selected for inclusion in the mixture based on the ability of the starch material to impart a specific organoleptic property to composition. Starches derived from various sources can be used. For example, major sources of starch include cereal grains (e.g., rice, wheat, and maize) and root vegetables (e.g., potatoes and cassava). Other examples of sources of starch include acorns, arrowroot, arracacha, bananas, barley, beans (e.g., favas, lentils, mung beans, peas, chickpeas), breadfruit, buckwheat, canna, chestnuts, colacasia, katakuri, kudzu, malanga, millet, oats, oca, Polynesian arrowroot, sago, sorghum, sweet potato, quinoa, rye, tapioca, taro, tobacco, water chestnuts, and yams. Certain starches are modified starches. A modified starch has undergone one or more structural modifications, often designed to alter its high heat properties. Some starches have been developed by genetic modifications, and are considered to be “genetically modified” starches. Other starches are obtained and subsequently physically (e.g., heat, cool water swelling, etc.), chemically, or enzymatically modified. For example, modified starches can be starches that have been subjected to chemical reactions, such as esterification, etherification, oxidation, depolymerization (thinning) by acid catalysis or oxidation in the presence of base, bleaching, transglycosylation and depolymerization (e.g., dextrinization in the presence of a catalyst), cross-linking, acetylation, hydroxypropylation, and/or partial hydrolysis. Enzymatic treatment includes subjecting native starches to enzyme isolates or concentrates, microbial enzymes, and/or enzymes native to plant materials, e.g., amylase present in corn kernels to modify corn starch. Other starches are modified by heat treatments, such as pregelatinization, dextrinization, and/or cold water swelling processes. Certain modified starches include monostarch phosphate, distarch glycerol, distarch phosphate esterified with sodium trimetaphosphate, phosphate distarch phosphate, acetylated distarch phosphate, starch acetate esterified with acetic anhydride, starch acetate esterified with vinyl acetate, acetylated distarch adipate, acetylated distarch glycerol, hydroxypropyl starch, hydroxypropyl distarch glycerol, starch sodium octenyl succinate.

In some embodiments, fillers are cellulose materials or cellulose derivatives. Cellulose can be provided, e.g., in powder (such as “microcrystalline” (MCC) or “ultrafine” (UFC)) form, such as ARBOCEL® powdered cellulose, e.g., including, but not limited to, ARBOCEL® UFC 100 or VIVAPUR® MCC, e.g., including, but not limited to, VIVAPUR® 105). One suitable particulate filler component for use in the products described herein is microcrystalline cellulose (“MCC”). The MCC may be synthetic or semi-synthetic, or it may be obtained entirely from natural celluloses. The MCC may be selected from the group consisting of AVICEL° grades PH-100, PH-102, PH-103, PH-105, PH-112, PH-113, PH-200, PH-300, PH-302, VIVACEL° grades 101, 102, 12, 20 and EMOCEL° grades 50M and 90M, and the like, and mixtures thereof. In some embodiment, the filler further comprises a cellulose derivative or a combination of such derivatives. In some embodiments, the mixture comprises from about 1 to about 10% of the cellulose derivative by weight, based on the total weight of the mixture, with certain embodiments comprising about 1 to about 5% by weight of cellulose derivative.

In certain embodiments, the cellulose derivative is a cellulose ether (including carboxyalkyl ethers), meaning a cellulose polymer with the hydrogen of one or more hydroxyl groups in the cellulose structure replaced with an alkyl, hydroxyalkyl, or aryl group. Non-limiting examples of such cellulose derivatives include methylcellulose, hydroxypropylcellulose (“HPC”), hydroxypropylmethylcellulose (“HPMC”), hydroxyethyl cellulose, and carboxymethylcellulose (“CMC”). In one embodiment, the cellulose derivative is one or more of methylcellulose, HPC, HPMC, hydroxyethyl cellulose, and CMC. In one embodiment, the cellulose derivative is HPC. These materials can be provided by various suppliers, e.g., Tic Gums (e.g., including, but not limited to, CMC pH 15, which can be employed in certain embodiments of the disclosed products). In certain embodiments, the film comprises a filler selected from the group consisting of polyols, dextrose, maltodextrin, and combinations thereof. In some embodiments, the composition comprises a filler selected from the group consisting of a sugar alcohol(s)/sugar substitute (e.g., sorbitol, maltitol, xylitol, isomalt, erythritol, and combinations thereof), glucose, maltose, maltotriose, maltodextrin, modified starches, and combinations thereof.

The quantity of filler optionally present in the edible, orally dissolvable film or oral product comprising such film as described herein may vary according to the desired properties. A filler, where included in the disclosed films, can be provided in an amount of about 1% to about 10% by weight, e.g., about 2% to about 5% by weight in some embodiments. Typically, in the disclosed films, the filler is present in an amount of less than about 15% by weight, less than 10% by weight, less than 8% by weight, less than 6% by weight, or less than 5% by weight, based on the total weight of the edible, orally dissolvable film.

The edible, orally dissolvable films can, in some embodiments, comprise one or more flavoring agents. As used herein, a “flavoring agent” or “flavorant” is any flavorful or aromatic substance capable of altering the sensory characteristics associated with the oral product. Examples of sensory characteristics that can be modified by the flavoring agent include taste, mouthfeel, moistness, coolness/heat, and/or fragrance/aroma. Flavoring agents may be natural or synthetic, and the character of the flavors imparted thereby may be described, without limitation, as fresh, sweet, herbal, confectionary, floral, fruity, or spicy. Specific types of flavors include, but are not limited to, vanilla, coffee, chocolate/cocoa, cream, mint, spearmint, menthol, peppermint, wintergreen, eucalyptus, lavender, cardamom, nutmeg, cinnamon, clove, cascarilla, sandalwood, honey, jasmine, ginger, anise, sage, licorice, lemon, orange, apple, peach, lime, cherry, strawberry, trigeminal sensates, terpenes, and any combinations thereof. See also, Leffingwell et al., Tobacco Flavoring for Smoking Products, R. J. Reynolds Tobacco Company (1972), which is incorporated herein by reference. Flavoring agents may comprise components such as terpenes, terpenoids, aldehydes, ketones, esters, and the like. In some embodiments, the flavoring agent is a trigeminal sensate. As used herein, “trigeminal sensate” refers to a flavoring agent which has an effect on the trigeminal nerve, producing sensations including heating, cooling, tingling, and the like. Non-limiting examples of trigeminal sensate flavoring agents include capsaicin, citric acid, menthol, Sichuan buttons, erythritol, and cubebol. Flavorings also may include components that are considered moistening, cooling or smoothening agents, such as eucalyptus. In some embodiments, the flavoring agents are sensates, e.g., selected from vanillyl butyl ether, spilanthol, alpha-hydroxy-sanshool, WS-3 (N-ethyl-5-methyl-2-(1-methylethyl)-cyclohexane carboxamide), WS-23 (N,2,3-trimethyl-2-(1-methylethyl)-butanamide), monomenthyl succinate, mono- and dimethylglutarate, and others. These flavors may be provided neat (i.e., alone) or in a composite, and may be employed as concentrates or flavor packages (e.g., spearmint and menthol, orange and cinnamon; lime, pineapple, and the like). Representative types of components also are set forth in U.S. Pat. No. 5,387,416 to White et al.; US Pat. App. Pub. No. 2005/0244521 to Strickland et al.; and PCT Application Pub. No. WO 05/041699 to Quinter et al., each of which is incorporated herein by reference. In some instances, the flavoring agent may be provided in a spray-dried form or a liquid form.

The flavoring agent generally comprises at least one volatile flavor component. As used herein, “volatile” refers to a chemical substance that forms a vapor readily at ambient temperatures (i.e., a chemical substance that has a high vapor pressure at a given temperature relative to a nonvolatile substance). Typically, a volatile flavor component has a molecular weight below about 400 Da, and often include at least one carbon-carbon double bond, carbon-oxygen double bond, or both. In one embodiment, the at least one volatile flavor component comprises one or more alcohols, aldehydes, aromatic hydrocarbons, ketones, esters, terpenes, terpenoids, or a combination thereof. Non-limiting examples of aldehydes include vanillin, ethyl vanillin, p-anisaldehyde, hexanal, furfural, isovaleraldehyde, cuminaldehyde, benzaldehyde, and citronellal. Non-limiting examples of ketones include 1-hydroxy-2-propanone and 2-hydroxy-3-methyl-2-cyclopentenone-1-one. Non-limiting examples of esters include allyl hexanoate, ethyl heptanoate, ethyl hexanoate, isoamyl acetate, and 3-methylbutyl acetate. Non-limiting examples of terpenes include sabinene, limonene, gamma-terpinene, beta-farnesene, nerolidol, thujone, myrcene, geraniol, nerol, citronellol, linalool, and eucalyptol. In one embodiment, the at least one volatile flavor component comprises one or more of ethyl vanillin, cinnamaldehyde, sabinene, limonene, gamma-terpinene, beta-farnesene, or citral. In one embodiment, the at least one volatile flavor component comprises ethyl vanillin.

The edible, orally dissolvable films can, in some embodiments, comprise pH adjusters or buffering agents. Examples of pH adjusters and buffering agents that can be used include, but are not limited to, metal hydroxides (e.g., alkali metal hydroxides such as sodium hydroxide and potassium hydroxide), and other alkali metal buffers such as metal carbonates (e.g., potassium carbonate or sodium carbonate), or metal bicarbonates such as sodium bicarbonate, and the like. Where present, the buffering agent is typically present in an amount less than about 5 percent based on the weight of the film, for example, from about 0.5% to about 5%, such as, e.g., from about 0.75% to about 4%, from about 0.75% to about 3%, or from about 1% to about 2% by weight, based on the total weight of the film. Non-limiting examples of suitable buffers include alkali metals acetates, glycinates, phosphates, glycerophosphates, citrates, carbonates, hydrogen carbonates, borates, or mixtures thereof.

In some embodiments, the edible, orally dissolvable films provided herein may further comprise a salt (e.g., alkali metal salt), typically employed in an amount sufficient to provide desired sensory attributes to the film. Non-limiting examples of suitable salts include sodium chloride, potassium chloride, ammonium chloride, flour salt, and the like.

A colorant may optionally be employed in amounts sufficient to provide the desired physical attributes to the film. Examples of colorants include various dyes and pigments, such as caramel coloring and titanium dioxide.

In some embodiments, the edible, orally dissolvable films provided herein can further comprise one or more sweeteners. Where present, the sweeteners can be any sweetener or combination of sweeteners, in natural or artificial form, or as a combination of natural and artificial sweeteners. Examples of natural sweeteners include isomaltulose, fructose, sucrose, glucose, maltose, mannose, galactose, lactose, stevia, honey, and the like. Examples of artificial sweeteners include sucralose, maltodextrin, saccharin, aspartame, acesulfame K, neotame and the like. In some embodiments, the sweetener comprises one or more sugar substitutes, e.g., alcohols. Sugar alcohols are polyols derived from monosaccharides or disaccharides that have a partially or fully hydrogenated form. Sugar alcohols have, for example, about 4 to about 20 carbon atoms and include erythritol, arabitol, ribitol, isomalt, maltitol, dulcitol, iditol, mannitol, xylitol, lactitol, sorbitol, and combinations thereof (e.g., hydrogenated starch hydrolysates).

The edible, orally dissolvable films provided herein can, in some embodiments, further comprise one or more processing aids. Processing aids can be, e.g., emulsifiers, dispersants, solubilizers, and/or surfactants.

For example, the edible, orally dissolvable films may, in some embodiments, comprise one or more surfactants and/or emulsifiers. Surfactants and emulsifiers can be used, in some embodiments to facilitate production of the films as outlined herein. Examples of surfactants and emulsifiers that can be used in the disclosed processes (and included in some embodiments of final edible, orally dissolvable films) include, but are not limited to, mono- and di-glycerides of fatty acids, glycerol esters, polyglycerol esters, lecithin, polyoxyethylene sorbitan fatty acid esters (polysorbates), propylene glycol fatty acid esters, and combinations thereof. Specific surfactants and emulsifiers include, e.g., Lumulse™ (e.g., Lumulse™ GMS K, which is an oil-soluble non-ionic surfactant used as a water-in-oil emulsifier, dispersant, carrier, and/or friction modifier), Polysorbate (e.g., Polysorbate 80), lecithin, glycerol monostearate, Lumisorb™ surfactants, including, but not limited to, PSMO-20-FGK (which can also function as a plasticizer), and combinations thereof.

In some embodiments, the edible, orally dissolvable films can further comprise a non-stick/anti-stick coating on one or both sides of the film. Non-stick/anti-stick coating materials can ensure that adjacent films do not stick to one another during production or storage (e.g., within a container). Suitable anti-stick coatings can comprise, e.g., oils and/or waxes and are known in the art. One example of a suitable anti-stick coating for one or both surfaces of the film provided herein is CAPOL®.

The edible, orally dissolvable films provided herein typically additionally comprise some moisture content. As such, the films comprise water, e.g., in an amount typically below about 20% by weight, below about 15% by weight, or below about 10% by weight. Generally, the average water content is about 1% to about 15%, e.g., about 2% to about 10% by weight based on the total weight of the edible, orally dissolvable film.

### Method of Producing Films

The edible, orally dissolvable films provided herein can be produced by a method generally involving mixing the components in a solvent (e.g., water), casting the resulting mixture into a film form, and drying the cast film form to remove at least a portion of the water therefrom. The method can be conducted in the absence of heat or, in some embodiments, heat can be employed at various stages of the process. The films provided herein are not limited to being produced in this manner; other methods for producing films, such as extrusion and the like can be used in various embodiments.

First, one or more of the components to be incorporated within the film is mixed together. Any mixing method that brings the mixture ingredients into intimate contact can be used, such as a mixing apparatus featuring an impeller or other structure capable of agitation. Examples of mixing equipment include casing drums, conditioning cylinders or drums, liquid spray apparatus, conical-type blenders, ribbon blenders, mixers available as FKM130, FKM600, FKM1200, FKM2000 and FKM3000 from Littleford Day, Inc., Plough Share types of mixer cylinders, Hobart mixers, v-shaped mixers, overhead mixers, and the like. See also, for example, the types of methodologies set forth in U.S. Pat. Nos. 4,148,325 to Solomon et al.; 6,510,855 to Korte et al.; and 6,834,654 to Williams, each of which is incorporated herein by reference. The amount of water in such mixtures is typically at least that amount sufficient to facilitate mixing and casting of the film composition. For example, the total solids content can, in some embodiments, be about 50% or less, about 40% or less, e.g., about 5% to about 50% or about 10% to about 40%.

The resulting mixture is then, in some embodiments, cast onto a flat carrier. The flat carrier can be any type of substrate or a forming surface. During the casting step, the mixture can be dispensed (e.g., poured or sprayed) onto the flat carrier and spread out over an area of the surface under the influence of gravity, and/or by spinning and/or using a suitable tool to assist in spreading. The forming surface is preferably suitable to form a uniform film thereon, and adapted to release a dried film therefrom. The forming surface can be formed from any suitable material including glass, stainless steel, Teflon, polyethylene, wax, and the like. The rate of dispensation can, in some embodiments, be adjusted to control the thickness of the film. During the dispensing, the viscosity of the mixture can be controlled, e.g., by controlling the temperature of the mixture. Preferably, a film of a predetermined thickness is formed on the forming surface.

The mixture can, in some embodiments, be metered onto the forming surface via a coating or casting system. A suitable apparatus for dispensing the mixture of components may comprise a manifold, one or more conduits, nozzles, valves, and/or blades, and the like.

The cast film is preferably air dried. In some embodiments, the cast film is first removed from the forming surface; in others, it is dried in situ on the forming surface, from which the dried film can be removed following the drying step. In some embodiments, a drying oven, a heat lamp, a vacuum dryer or any other suitable drying system can be employed to dry the cast film. After being cast and dried, the resulting film can be cut into desired sizes/shapes, as outlined herein below and packaged for use and/or combined with other materials to form various types of oral products.

In one embodiment, all components to be included within a film are mixed together at room temperature in water. The film composition is cast onto a flat carrier and dried (e.g., to about 1-10% moisture content). In one embodiment, water is heated and combined with the binder; the resulting mixture is mixed. Plasticizer is then added, along with any remaining ingredients, and the mixture is mixed until smooth. The film composition is cast onto a flat carrier and dried at elevated temperature (e.g., at least about 50° C., at least about 70° C., at least about 80° C., or at least about 90° C., e.g., at about 100° C. or about 50° C. to about 100° C. or about 80° C. to about 100° C.).

In some embodiments, materials other than those expressly described herein (e.g., microcapsules, active components, material comprising finely ground film) can be incorporated during the process of preparing an edible, orally dissolvable film as provided herein. For example, in some embodiments, such additional materials can be added, e.g., to the surface of a moist film during production and thereby incorporated within the resulting matrix. In some embodiments, the film provided herein can have a perforated porous structure added to the film, e.g., via the use of a pin or brush roll during converting of the film.

### Oral Products Comprising Films

### Oral Film

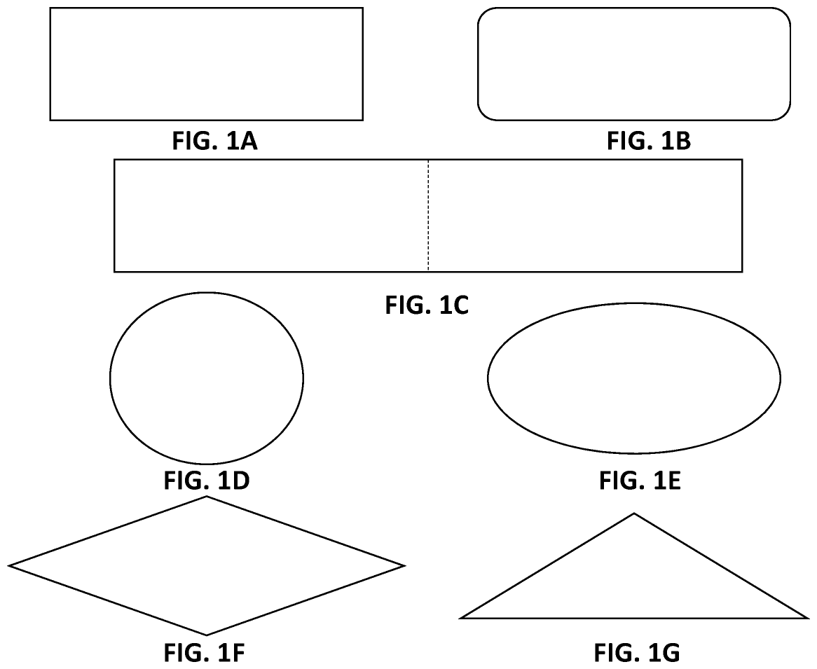

In certain embodiments, an oral product is provided that consists essentially of one or more edible, orally dissolvable films as provided herein (i.e., an oral film). The edible, orally dissolvable films can be translucent or opaque. In some such embodiments, the edible, orally dissolvable film is thus in a size and shape suitable to fit within the oral cavity. For example, the orally dissolvable film can be a quadrilateral (e.g., a rectangle, square, rhombus, or parallelogram), or can be, e.g., oval, circular, trapezoidal, or triangular in shape. In some embodiments, the edible, orally dissolvable film is provided in a longer ribbon/strip, e.g., with perforations to allow for a single portion to be removed and placed within the oral cavity. Some examples of suitable shapes for oral products in the form of films are provided herein in FIGS. 1A to 1G.

Examples of suitable piece sizes for use (whether single portion or perforated portion of a larger portion of material) are, for example, about 10 to about 40 mm in any dimension (e.g., length and width, diameter, etc. In one embodiment, the piece is rectangular (with angular or rounded edges) and the piece has a length of about 30-40 mm and a width of about 10-15 mm. The basis weight of a piece of the oral product (consisting essentially of one or more orally dissolvable films) is generally about 10 mg to about 500 mg, e.g., about 25 mg to about 500 mg or about 50 mg to about 500 mg, e.g., 100 mg to about 400 mg, e.g., for a piece sized 33 mm×11 mm. Such pieces can have a basis weight of about 10 mg, about 20 mg, about 25 mg, about 30 mg, about 35 mg, about 40 mg, about 45 mg, or about 50 mg with the referenced dimensions in particular, non-limiting embodiments. Particularly in embodiments wherein the edible, orally dissolvable film provided herein is employed in combination with another material, e.g., fleece, it can advantageously have lower basis weight (e.g., about 25 mg or about 50 mg); in some embodiments, the edible, orally dissolvable film provided herein, when employed as a stand-alone oral product, advantageously exhibits a basis weight that is somewhat higher. It may, in some embodiments, be advantageous to provide an edible, orally dissolvable film that is of higher caliper than, e.g., breath strips, such that the film is parkable within the oral cavity for a longer period of time.

In various embodiments, the film can be adapted to or configured to at least partially dissolve or completely dissolve in about 5 minutes or longer, about 15 minutes or longer, about 30 minutes or longer, or about an hour or longer. In certain embodiments, the film can be configured to at least partially dissolve or completely dissolve in no less than 30 minutes, no less than 45 minutes, or no less than an hour. In some embodiments, the film can be configured to at least partially dissolve or completely dissolve in a time of about 30 seconds or more, e.g., 30 seconds to about 30 minutes, about 1 minute to about 25 minutes, about 5 minutes to about 20 minutes, or about 5 minutes to about 15 minutes.

In some embodiments, the basis weight of the film product can be varied by combining two or more films, e.g., laminating two or more films to one another. Various methods of laminating orally edible films are known and can be employed according to the present disclosure. As such, in some embodiments, an oral product is provided consisting of or consisting essentially of a single film; in some embodiments, an oral product is provided consisting of or consisting essentially of two laminated films;

in some embodiments, an oral product is provided consisting of or consisting essentially of three laminated films; in some embodiments, an oral product is provided consisting of or consisting essentially of four (or more) laminated films. Each such laminated film can be the same or can be different in composition and/or size and/or shape.

In some embodiments, an oral product that consists of or consists essentially of one or more films (i.e., a single film or a laminated film) can have a texture on one or both surfaces thereof (i.e., top and bottom surfaces) that can be described as smooth. In some embodiments, the single film or laminated film can have a texture on one or both surfaces thereof that can be described as textured. For example, in some embodiments, one or both surfaces of the film(s) can comprise raised portions (e.g., dots or stripes) that can be effective, e.g., for grip and piece control during use. The raised portions can, in some embodiments, comprise the same material as the remainder of the film(s) or may be different. In some embodiments, the raised portions are introduced during production of the film itself, or can be a post-production modification, e.g., such that the raised portions introduced onto one or more surfaces of the film after it is formed, e.g., via embossing or any other type of method to introduce texture to the surface(s).

In other embodiments, the orally dissolvable film is incorporated within another type of oral product, i.e., the oral product comprises one or more additional components in addition to the orally dissolvable film.

### Film-based Pouch

In some embodiments, the edible, orally dissolvable film provided herein can be used as a component of a film-based pouch, e.g., to contain a material adapted for oral use therein (e.g., a particulate mixture adapted for oral use). For example, an example oral product can comprise a film as described herein as an outer water-permeable container in the form of a pouch which contains a particulate mixture adapted for oral use therein. The orientation, size, and type of outer water-permeable film-based pouch and the type and nature of the composition adapted for oral use that are illustrated herein are not to be construed as limiting thereof.

In some embodiments, the film used for such film-based pouches does not comprise an active ingredient. In other embodiments, the film can comprise one or more active ingredients as outlined herein above.

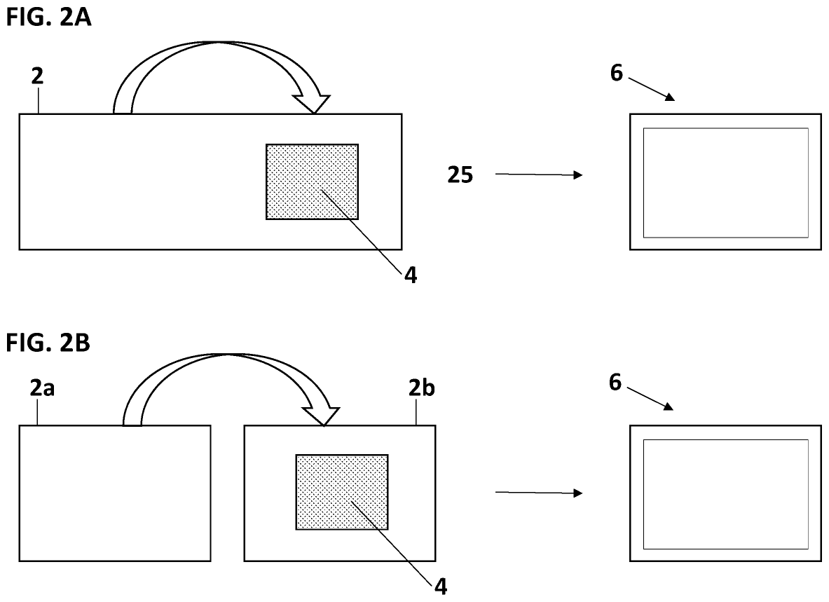

As shown in FIG. 2A, in one embodiment, the film 2 can be provided as a single film that is folded over the material adapted for oral use 4 (e.g., particulate mixture adapted for oral use) and sealed on all other edges to contain the particulate mixture 4, giving film-based pouched product 6. In another embodiment, as shown in FIG. 2B, two films 2a and 2b (which can be the same or different) can be used as two surfaces of the pouch by laying down one film (e.g., 2b), placing the material adapted for oral use 4 on the surface of that film (2b), placing a second film, 2a, on top, and sealing all edges thereof to contain the particulate mixture 4, giving film-based pouched product 6.

The material adapted for oral use 4 contained within such a film-based pouch 6 is not particularly limited, and can comprise any filling composition, including those that can be included within conventional (e.g., fleece-based) pouched products. Such compositions are generally mixtures, e.g., particulate mixtures, of two or more components and as such, the compositions are, in some cases, referenced herein below as “mixtures.” Such mixtures can comprise, e.g., one or more active ingredients and/or one or more flavorants, and various other optional ingredients (e.g., fillers, pH adjusters/buffering agents, colorants, humectants, salts, sweeteners, and the like). Various additives can be included in the Eisclosed mixture; for example, the mixture can be processed, blended, formulated, combined and/or mixed with other materials or ingredients. The additives can be artificial, or can be obtained or derived from herbal or biological sources. Examples of further types of additives include thickening or gelling agents (e.g., fish gelatin), emulsifiers, oral care additives (e.g., thyme oil, eucalyptus oil, and zinc), preservatives (e.g., potassium sorbate and the like), zinc or magnesium salts selected to be relatively water soluble for compositions with greater water solubility (e.g., magnesium or zinc gluconate) or selected to be relatively water insoluble for compositions with reduced water solubility (e.g., magnesium or zinc oxide), disintegration aids, or combinations thereof. See, for example, those representative components, combination of components, relative amounts of those components, and manners and methods for employing those components, set forth in U.S. Pat. No. 9,237,769 to Mua et al., U.S. Pat. No. 7,861,728 to Holton, Jr. et al., US Pat. App. Pub. No. 2010/0291245 to Gao et al., and US Pat. App. Pub. No. 2007/0062549 to Holton, Jr. et al., each of which is incorporated herein by reference. Typical inclusion ranges for such additional additives can vary depending on the nature and function of the additive and the intended effect on the final mixture, with an example range of up to about 10% by weight, based on total weight of the mixture (e.g., about 0.1 to about 5% by weight).

Certain components that can advantageously be included in the mixtures within certain embodiments of the pouches provided herein are outlined generally below; however, it is to be understood that the discussion below is not intended to be limiting of the components that can be incorporated within the disclosed pouches. In certain embodiments, the composition within the film-based pouch provided herein includes only saliva soluble materials. In various embodiments, the composition within the film-based can be orally dissolvable. The composition can be configured to provide sustained release of active ingredient(s) and/or flavorant(s) upon contact with the saliva in the mouth of a user. After use, the entire composition, and in certain embodiments, the entire film-based pouched material originally housing the composition, can be dissolved and orally ingested by the user such that there is nothing left of the pouched product to remove from the mouth of the user.

In various embodiments, the composition within the film-based pouch can be adapted to or configured to at least partially dissolve or completely dissolve in about 5 minutes or longer, about 15 minutes or longer, about 30 minutes or longer, or about an hour or longer. In certain embodiments, the composition can be configured to at least partially dissolve or completely dissolve in no less than 30 minutes, no less than 45 minutes, or no less than an hour. In some embodiments, the composition can be configured to at least partially dissolve or completely dissolve in a time of about 30 seconds to about 30 minutes, about 1 minute to about 25 minutes, about 5 minutes to about 20 minutes, or about 5 minutes to about 15 minutes.

The material within the pouches as described herein can include at least one particulate filler component. Such particulate filler components may fulfill multiple functions, such as enhancing certain organoleptic properties such as texture and mouthfeel, enhancing cohesiveness or compressibility of the product, and the like. The filler components are particulate materials and can include components as described above with respect to the optional filler component of the edible, orally dissolvable film provided herein, e.g., cellulosic non-tobacco plant material and derivatives thereof including, but not limited to, cereal grains, sugar beet, bran fiber, starches, natural cellulose, modified cellulosic materials, maltodextrin, dextrose, calcium carbonate, calcium phosphate, lactose, sugar substitutes (e.g., mannitol, xylitol, and sorbitol). Combinations of fillers can also be used. In some embodiments, the filler component can be described as a particulate material. As used herein, the term “particulate” refers to a material in the form of a plurality of individual particles, some of which can be in the form of an agglomerate of multiple particles, wherein the particles have an average length to width ratio less than 2:1, such as less than 1.5:1, such as about 1:1. In various embodiments, the particles of a particulate material can be described as substantially spherical or granular (e.g., in the form of beads).

The amount of particulate filler component within a film-based pouched product can vary, but is typically up to about 75 percent of the material contained within the pouch by weight (i.e., the mixture), based on the total weight of the mixture. A typical range of particulate filler material (e.g., MCC) within the mixture can be from about 10 to about 75 percent by total weight of the mixture, for example, from about 10, about 15, about 20, about 25, or about 30, to about 35, about 40, about 45, or about 50 weight percent (e.g., about 20 to about 50 weight percent or about 25 to about 45 weight percent). In certain embodiments, the amount of particulate filler material is at least about 10 percent by weight, such as at least about 20 percent, or at least about 25 percent, or at least about 30 percent, or at least about 35 percent, or at least about 40 percent, based on the total weight of the mixture.

The water content of the particulate mixture within the film-based pouched product described herein, prior to use by a consumer of the product, may vary according to the desired properties. Typically, the mixture, as present within the product prior to insertion into the mouth of the user, is less than about 60 percent by weight of water, and generally is from about 1 to about 60% by weight of water, for example, from about 5 to about 55, about 10 to about 50, about 20 to about 45, or about 25 to about 40 percent water by weight, including water amounts of at least about 5% by weight, at least about 10% by weight, at least about 15% by weight, and at least about 20% by weight.

The particulate mixture can, in some embodiments, further comprise one or more flavoring agents. The amount of flavoring agent utilized in the mixture can vary, but is typically up to about 10 weight percent, and certain embodiments are characterized by a flavoring agent content of at least about 0.1 weight percent, such as about 0.5 to about 10 weight percent, about 1 to about 6 weight percent, or about 2 to about 5 weight percent, based on the total weight of the mixture. The amount of flavoring agent present within the mixture may vary over a period of time (e.g., during a period of storage after preparation of the mixture). For example, certain volatile components present in the mixture may evaporate or undergo chemical transformations, leading to a reduction in the concentration of one or more volatile flavor components. In one embodiment, a concentration of one or more of the at least one volatile flavor components present is greater than a concentration of the same one or more volatile flavor components present in a control pouched product which does not include the one or more organic acids, after the same time period. Without wishing to be bound by theory, it is believed that the same mechanisms responsible for loss of whiteness result in a gradual decline in certain volatile components in the flavoring (e.g., aldehydes, ketones, terpenes). Therefore, a decline in the presence of these volatile components leading to the discoloration over time may be expected to diminish the sensory satisfaction associated with products subject to such a degradation process.

In some embodiments, the mixture within the film-based pouch may further comprise a salt (e.g., alkali metal salts), typically employed in an amount sufficient to provide desired sensory attributes to the mixture. Non-limiting examples of suitable salts include sodium chloride, potassium chloride, ammonium chloride, flour salt, and the like. When present, a representative amount of salt is at least about 0.5 percent by weight, at least about 1.0 percent by weight, or at least about 1.5 percent by weight, but will typically make up about 10 percent or less of the total weight of the mixture, or about 7.5 percent or less or about 5 percent or less (e.g., about 0.5 to about 5 percent by weight).

The mixture within the film-based pouch typically further comprises one or more sweeteners, as referenced above with respect to the edible, orally dissolvable film provided herein. When present, a representative amount of sweetener may make up from about 0.1 to about 20 percent or more of the of the mixture by weight, for example, from about 0.1 to about 1%, from about 1 to about 5%, from about 5 to about 10%, or from about 10 to about 20% of the mixture on a weight basis, based on the total weight of the mixture.

A binder (or combination of binders) may be employed in certain embodiments as a component of the particulate material within a film-based pouch, in amounts sufficient to provide the desired physical attributes and physical integrity to the mixture. Binders also often function as thickening or gelling agents. Typical binders can be organic or inorganic, or a combination thereof. Representative binders include modified cellulose, povidone, sodium alginate, starch-based binders, pectin, carrageenan, pullulan, zein, gums, and the like, and combinations thereof. A binder may be employed in amounts sufficient to provide the desired physical attributes and physical integrity to the mixture. In some embodiments, the binder of the particulate material within the pouch comprises pectin or carrageenan or combinations thereof. The amount of binder utilized in the mixture can vary, but is typically up to about 30 weight percent, and certain embodiments are characterized by a binder content of at least about 0.1% by weight, such as about 1 to about 30% by weight, or about 5 to about 10% by weight, based on the total weight of the mixture.

In certain embodiments, one or more humectants may be employed in the mixture. Examples of humectants include, but are not limited to, glycerin, propylene glycol, and the like. Where included, the humectant is typically provided in an amount sufficient to provide desired moisture attributes to the mixture. Further, in some instances, the humectant may impart desirable flow characteristics to the mixture. When present, a humectant will typically make up about 5% or less of the weight of the mixture (e.g., from about 0.5 to about 5% by weight). When present, a representative amount of humectant is about 0.1% to about 1% by weight, or about 1% to about 5% by weight, based on the total weight of the mixture.

A colorant may be employed in amounts sufficient to provide the desired physical attributes to the mixture. The amount of colorant utilized in the mixture can vary, but when present is typically up to about 3 weight percent, such as from about 0.1%, about 0.5%, or about 1%, to about 3% by weight, based on the total weight of the mixture.

The material within the pouch may or may not comprise an active ingredient. Where they are included, the active ingredient(s) within the pouch can be the same as or different than the active ingredient(s) optionally contained in the film-based pouch component. Examples of active ingredients that can be included within the composition in the pouch include, but are not limited to, the active ingredients described herein above.

It is noted that such film-based pouches are not limited to containing a particulate mixture. In some embodiments, the material adapted for oral use 4 within the pouch can be, e.g., a further film-based material or can be a liquid or gel material.

One or more of the film layers of a film-based pouched product can, in some embodiments, include at least one perforation, e.g., a plurality of perforations. Perforations can aid in entanglement of the edible, orally dissolvable films and the one or more materials encapsulated therein (providing, e.g., better inter-layer adhesion and a more structurally stable product).

Film on fleece-based pouch

In one embodiment, a pouched product is provided that comprises an orally dissolvable film as described herein as a component of a fleece-based pouch (e.g., on at least a portion of an inner and/or outer surface thereof or within the cavity of the pouch, as outlined herein below). A pouched product generally comprises a mixture of one or more components, disposed within a moisture-permeable container (e.g., a water-permeable pouch). Exposure to saliva causes some of the components of the mixture therein (e.g., flavoring agents and/or active ingredients) to pass through e.g., the water-permeable pouch and provide the user with flavor and satisfaction, and the user is not required to spit out any portion of the mixture. After about 10 minutes to about 60 minutes, typically about 15 minutes to about 45 minutes, of use/enjoyment, substantial amounts of the mixture have been ingested by the human subject, and the pouch may be removed from the mouth of the consumer for disposal. Preferred pouch materials for products described herein may be designed and manufactured such that under conditions of normal use, a significant amount of the contents of the formulation within the pouch permeate through the pouch material prior to the time that the pouch undergoes loss of its physical integrity.

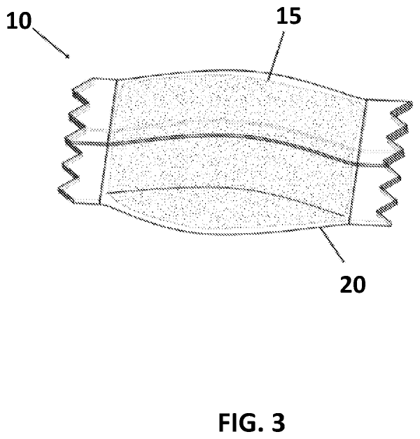

An example of a pouched product generally is illustrated in FIG. 3, wherein the example pouched product 10 can comprise an outer water-permeable container 20 in the form of a pouch which contains a composition 15 adapted for oral use. Composition 15 can be, in some embodiments, a particulate material or can be an edible, orally dissolvable film as described herein. In various embodiments, a moisture-permeable packet or pouch can act as a container for use of the composition (e.g., particulate material or film) within. For example, the pouch provides a liquid-permeable container of a type that may be considered to be similar in character to the mesh-like type of material that is used for the construction of a tea bag. If desired, flavoring ingredients, disintegration aids, and other desired components, may be incorporated within, or applied to, the pouch material. The general composition/construction of such packets or pouches, such as the container pouch 20 in the embodiment illustrated in FIG. 3, may be varied as noted herein. For example, suitable packets, pouches or containers of the type used for the manufacture of smokeless tobacco products, which can be modified according to the present disclosure, are available under the tradenames CatchDry, Ettan, General, Granit, Goteborgs Rape, Grovsnus White, Metropol Kaktus, Mocca Anis, Mocca Mint, Mocca Wintergreen, Kicks, Probe, Prince, Skruf and TreAnkrare. A pouch type of product similar in shape and form to various embodiments of a pouched product described herein is commercially available as ZONNIC (distributed by Niconovum AB). Additionally, pouch type products generally similar in shape and form to various embodiments of a pouched product are set forth as snuff bag compositions E-J in Example 1 of PCT WO 2007/104573 to Axelsson et al., which is incorporated herein by reference, which are produced using excipient ingredients and processing conditions that can be used to manufacture pouched products as described herein.

The pouch itself can be formed from a fleece material, e.g., a fibrous nonwoven web. As used herein, the term “fiber” is defined as a basic element of textiles. Fibers are often in the form of a rope- or string-like element. As used herein, the term “fiber” is intended to include fibers, filaments, continuous filaments, staple fibers, and the like. The term “multicomponent fibers” refers to fibers that comprise two or more components that are different by physical or chemical nature, including bicomponent fibers. Specifically, the term “multicomponent fibers” includes staple and continuous fibers prepared from two or more polymers present in discrete structured domains in the fiber, as opposed to blends where the domains tend to be dispersed, random or unstructured.

A “fleece material” as used herein may be formed from various types of fibers, as described in more detail herein below, capable of being formed into a traditional fleece fabrics or other traditional pouch materials. For example, fleece materials may be provided in the form of a woven or nonwoven fabric. Suitable types of fleece materials, for example, are described in U.S. Pat. No. 8,931,493 to Sebastian et al.; and US Patent App. Pub. Nos. 2015/0128978 to Sebastian et al., 2016/0000140 to Sebastian et al., and US Patent App. Pub. No. 2016/0073689 to Sebastian et al.; which are all incorporated herein by reference.

The term “nonwoven” is used herein in reference to fibrous materials, webs, mats, batts, or sheets in which fibers are aligned in an undefined or random orientation. The nonwoven fibers are initially presented as unbound fibers or filaments. An important step in the manufacturing of nonwovens involves binding the various fibers or filaments together. The manner in which the fibers or filaments are bound can vary, and include thermal, mechanical and chemical techniques that are selected in part based on the desired characteristics of the final product, as discussed in more detail herein below.

In various embodiments, the pouch material can be dissolvable (i.e., orally ingestible) such that under conditions of normal use (i.e., upon contact with saliva in the mouth of a user), the pouch material dissolves. Preferably, the pouch material will dissolve after a significant amount of the soluble components of the composition within the pouch (e.g., active ingredient(s) and/or flavorant(s)) permeate through the pouch material into the mouth of the user. For example, the pouch material can be configured to dissolve at a rate such that the pouch material holds the composition together for a period of time sufficient to allow for the release of substantially all water soluble components. As described in more detail below, in certain embodiments, the composition within the pouch material can also be dissolvable. In such embodiments, the pouch material can be configured to dissolve at a rate similar to the rate at which the composition dissolves. In certain embodiments, the pouch material can be adapted to or configured to at least partially dissolve or completely dissolve in about 5 minutes or longer, about 15 minutes or longer, about 30 minutes or longer, or about an hour or longer. In certain embodiments, the pouch material can be adapted to or configured to at least partially dissolve or completely dissolve in no less than 30 minutes, no less than 45 minutes, or no less than an hour. In some embodiments, the pouch material may be adapted to or configured to at least partially dissolve or completely dissolve in a time of about 30 seconds to about 30 minutes, about 1 minute to about 25 minutes, about 5 minutes to about 20 minutes, or about 5 minutes to about 15 minutes. Without being limited by theory, a pouched product comprising a dissolvable pouch material can provide environmental advantages.

In various embodiments, dissolvable pouch materials can include, but are not limited to, spun or nonwoven alginate fibers, gluten fibers, mini-perforated flat sheets derived from alginate, carrageenan, and other polymer binders, and combinations thereof. Without being limited by theory, the dissolution rate of the pouch material can be controlled, in part, by the use of cross-linking technology between alginate or pectin and calcium salts, for example. In certain embodiments, the dissolvable pouch material can include fast dissolving fibers formed using an electrospinning process (e.g., solution-based electrospinning) with hydrophilic polymers. See, e.g., the techniques and fibers disclosed in Asawahame, Chawalinee et al., Formation of Orally Fast Dissolving Fibers Containing Propolis by Electrospinning Technique, Chiang Mai J. Sci. 2015; 42(2), p. 469-480, which is herein incorporated by reference in its entirety.

In some embodiments, the fibers within the fleece material may include, but are not limited to, a polymer selected from the group consisting of polyglycolic acid, polylactic acid, polyhydroxyalkanoates, polycaprolactone, polybutylene succinate, polybutylene succinate adipate, and copolymers thereof. In some embodiments, the fibers within the fleece material may be selected from the groups consisting of wool, cotton, fibers made of cellulosic material, such as regenerated cellulose, cellulose acetate, cellulose triacetate, cellulose nitrate, ethyl cellulose, cellulose acetate propionate, cellulose acetate butyrate, hydroxypropyl cellulose, methyl hydroxypropyl cellulose, protein fibers, and the like. See also, the fiber types set forth in US Pat. Appl. Pub. No. 2014/0083438 to Sebastian et al., which is incorporated by reference herein. In various embodiments, the pouch material can include a polymer selected from the group consisting of polyvinylpyrrolidone, polyvinyl alcohol, and combinations thereof.

Regenerated cellulose fibers can be particularly advantageous, and are typically prepared by extracting non-cellulosic compounds from wood, contacting the extracted wood with caustic soda, followed by carbon disulfide and then by sodium hydroxide, giving a viscous solution. The solution is subsequently forced through spinneret heads to create viscous threads of regenerated fibers. Example methods for the preparation of regenerated cellulose are provided in U.S. Pat. No. 4,237,274 to Leoni et al; U.S. Pat. No. 4,268,666 to Baldini et al; U.S. Pat. No. 4,252,766 to Baldini et al.; U.S. Pat. No. 4,388,256 to Ishida et al.; U.S. Pat. No. 4,535,028 to Yokogi et al.; U.S. Pat. No. 5,441,689 to Laity; U.S. Pat. No. 5,997,790 to Vos et al.; and U.S. Pat. No. 8,177,938 to Sumnicht, which are incorporated herein by reference. The manner in which the regenerated cellulose is made is not limiting, and can include, for example, both the rayon and the Lyocell processes. Various suppliers of regenerated cellulose are known, including Lenzing (Austria), Cordenka (Germany), Aditya Birla (India), and Daicel (Japan).

The form of the fibers used in the nonwoven web according to the present disclosure can vary, and include fibers having any type of cross-section, including, but not limited to, circular, rectangular, square, oval, triangular, and multilobal. In certain embodiments, the fibers can have one or more void spaces, wherein the void spaces can have, for example, circular, rectangular, square, oval, triangular, or multilobal cross-sections. As noted previously, the fibers can be selected from single-component (i.e., uniform in composition throughout the fiber) or multicomponent fiber types including, but not limited to, fibers having a sheath/core structure and fibers having an islands-in-the-sea structure, as well as fibers having a side-by-side, segmented pie, segmented cross, segmented ribbon, or tipped multilobal cross-sections.

The physical parameters of the fibers present in the nonwoven web can vary. For example, the fibers used in the nonwoven web can have varying size (e.g., length, denier per filament (dpf)) and crimp characteristics. In some embodiments, fibers used in the nonwoven web can be nano fibers, sub-micron fibers, and/or micron-sized fibers. In certain embodiments, fibers of the nonwoven webs useful herein can measure about 1.5 dpf to about 2.0 dpf, or about 1.6 dpf to about 1.90 dpf. In various embodiments, each fiber can measure about 4-10 crimps per cm, or about 5-8 crimps per cm. In some embodiments, each fiber can be a continuous filament fiber. In certain embodiments, each fiber can be a staple fiber. Each fiber length can measure about 35 mm to about 60 mm, or about 38 mm to about 55 mm, for example. It can be advantageous for all fibers in the nonwoven web to have similar fiber size and crimp attributes to ensure favorable blending and orientation of the fibers in the nonwoven web.

The fibrous webs can have varying thicknesses, porosities and other parameters. The nonwoven web can be formed such that the fiber orientation and porosity of the pouched product formed therefrom can retain the composition adapted for oral use that is enclosed within the outer water-permeable pouch, but can also allow the flavors of the composition to be enjoyed by the consumer. For example, in some embodiments, the fibrous webs can have a basis weight of about 20 gsm to about 35 gsm, or about 25 gsm to about 30 gsm. In a preferred embodiment, the fibrous web can have a basis weight of about 28 gsm. Basis weight of a fabric can be measured using ASTM D3776/D3776M-09a (2013) (Standard Test Methods for Mass Per Unit Area (Weight) of Fabric), for example. In various embodiments, the fibrous web can have a thickness of about 0.1 mm to about 0.15 mm (e.g., about 0.11 mm). The fibrous web can have an elongation of about 70% to about 80%, e.g., about 78%. In some embodiments, the fibrous web can have a peak load of about 4 lbs. to about 8 lbs., e.g., about 5.5 lbs. Elongation and breaking strength of textile fabrics can be measured using ASTM D5034-09(2013) (Standard Test Method for Breaking Strength and Elongation of Textile Fabrics (Grab Test)), for example. In various embodiments, the fibrous web can have a Tensile Energy Absorption (TEA) of about 35 to about 40, e.g., about 37. In certain embodiments, the fibrous web can have a porosity of greater than about 10,000 ml/min/cm². TEA can be measured, for example, as the work done to break the specimen under tensile loading per lateral area of the specimen. Porosity, or air permeability of textile fabrics can be measured using ASTM D737-04(2012) (Standard Test method for Air Permeability of Textile Fabrics), for example.

The orientation, size, and type of outer water-permeable pouch and the type and nature of the composition adapted for oral use that are illustrated herein are not construed as limiting thereof. The edible, orally dissolvable film associated with the fleece according to the present disclosure (not shown in FIG. 3) can be associated, e.g., with an inner and/or outer surface of the outer water-permeable container 20. For example, the orally dissolvable film can be provided as a full coating on one or more inner and/or outer surfaces of the container 20. In some embodiments, the edible, orally dissolvable film is provided in discrete sections on the inner and/or outer surface of the container 20. For example, the film can be provided in the form of one or more strips/stripes on the inner and/or outer surface, one or more circles/dots (e.g., raised dots) on the inner and/or outer surface, a lattice-type structure on the inner and/or outer surface, or in other patterns on the inner and/or outer surface.

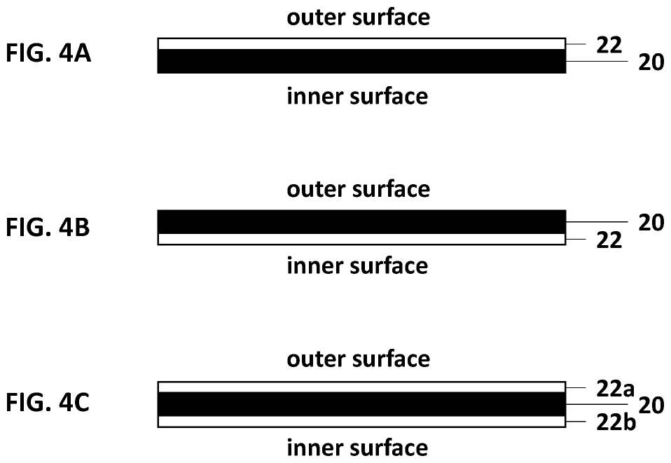

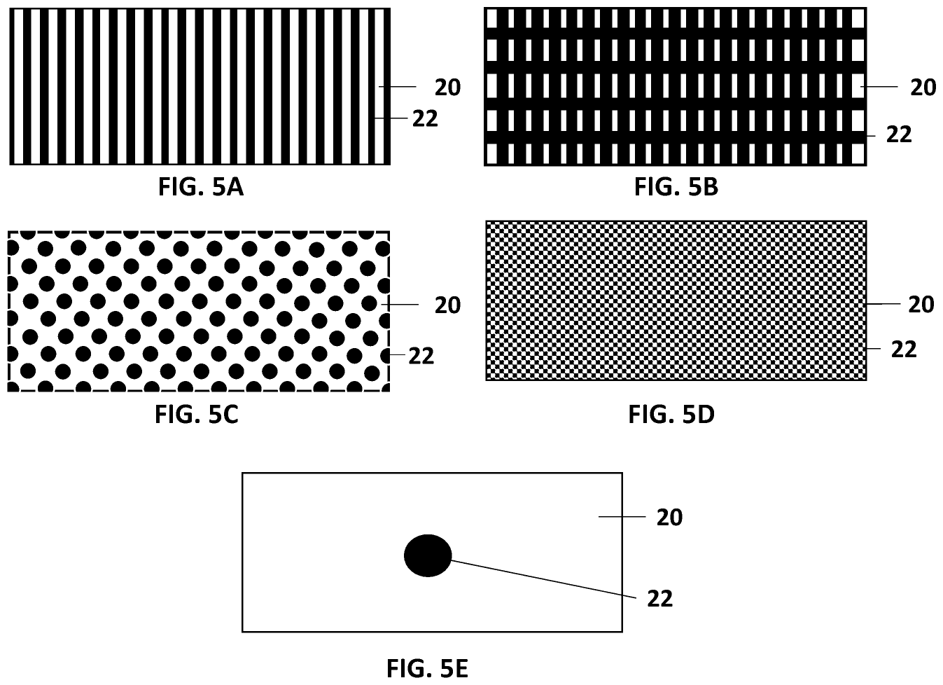

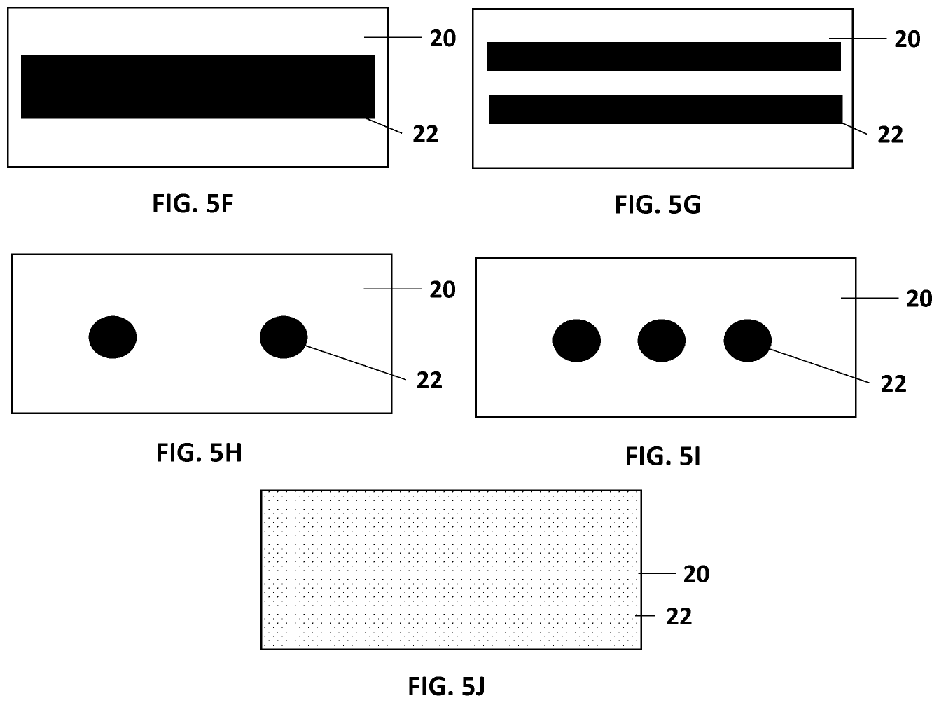

Certain examples of films that are associated with the inner and/or outer surface of a container 20 or within the container 20 are shown in cross-section in FIGS. 4A, 4B, and 4C, wherein 20 represents the outer water-permeable container (e.g., comprising a fleece material) and 22 represents the edible, orally dissolvable film (with 22a and 22b in FIG. 4C representing two different film components, which can be the same or different). The films can be associated with the inner and/or outer surface of container 20 on both a front and back side of the product 10 or only on one side of the product 10. Other non-limiting examples of patterned films associated with the inner and/or outer surface of a container 20 are shown in surface (e.g., top-down) view in FIGS. 5A, 5B, 5C, 5D, 5E, 5F, 5G, 5H, 5I, and 5J, where the patterns afforded by the film can be present on the inside and/or outside surface of the container 20, and can be present on one or both sides of the product (e.g., on one or both sides of Product 10, as illustrated in FIG. 3). Where more than one film component 22 is present on the surface, each such film component can be the same or different. In some specific embodiments, multiple film components can be present on a surface, each containing a different flavorant and/or active agent or each containing a different amount of flavorant and/or active agent. For example, the multiple strips in FIGS. 5A, 5B, and 5G and the multiple dots in FIGS. 5C, 5H, 5I, and 5J can, in some embodiments, include one or more different components (e.g., different flavorants) with respect to one another.

The effect of the film on the pouched product can vary. In some embodiments, it can provide a unique sensory effect, e.g., provided by raised film structures (e.g., dots or strips/stripes) on one or more outer surfaces of the oral product that come into contact with the oral cavity during use. In some embodiments, the film can provide for a unique visual effect. For example, the film can be colored and can endow the oral product with colored portions, e.g., throughout the surface (such as confetti distributed within the material).

In some embodiments, a heat sealable binder coating or a binder material (e.g., a coating or other additive) may be added to the fibers prior to, during, or after forming the fleece material. As used herein, “heat sealable binder coatings” refers to coating materials, such as acrylic polymer compositions, applied to a substrate (e.g., a nonwoven web or fleece material) and which are capable of sealing seams of individual pouches upon heating. In some embodiments, a binder material can be added to the web fibers before or during the laying of the fibrous web (i.e., before the fibrous web is bonded to form a fleece material). In certain embodiments, a binder material can be added to the fleece material after it has been formed. In various embodiments, the binder material is in the form of a liquid coating. In certain embodiments, a binding powder can be applied to the fleece material. For example, powdered polyethylene can be used as a binder material. The liquid or powder coating can be applied, for example, between layers of fibers when cross-laying, air laying, or as an after treatment. A short exposure in an oven is sufficient to melt and fuse the binder material.

The oral composition within the fleece-based pouch can vary and can, in some embodiments, comprise a film as provided herein (which can be simply placed within the cavity before or after production of the pouched product. In some embodiments, the oral composition within the fleece-based pouch is a mixture (e.g., a particulate mixture) as described above with respect to film-based pouches. The composition within the fleece-based pouch typically includes one or more active ingredients, as described herein above. In certain embodiments, the composition within the pouched product can comprise a lozenge- or pastille-type composition which has then been ground prior to insertion into the fleece material. The manners and methods used to formulate and manufacture the lozenge/pastille product can vary. For example, the compositions can be prepared via any method commonly used for the preparation of hard boiled confections. Exemplary methods for the preparation of hard confections can be found, for example, in LFRA Ingredients Handbook, Sweeteners, Janet M. Dalzell, Ed., Leatherhead Food RA (December 1996), pp. 21-44, which is incorporated herein by reference. See also, U.S. Pat. Pub. No. 2018/0228204 to Holton Jr. et al., which is herein incorporated by reference in its entirety.

The means of producing the fleece pouch material can vary. Web formation can be accomplished by any means known in the art. Nonwoven web formation will typically involve a carding step, which involves deposition of the fibers onto a surface followed by aligning/blending the fibers in a machine direction. Thereafter, the fibrous web is typically subjected to some type of bonding/entanglement including, but not limited to, thermal fusion or bonding, mechanical entanglement, chemical adhesive, or a combination thereof. In one embodiment, the fibrous web is bonded thermally using a calendar (which can provide flat or point bonding), steam jet bonding, or a thru-air oven. Additional bonding methods include ultrasonic bonding and crimping. In some embodiments, needle punching is utilized, wherein needles are used to provide physical entanglement between fibers. In one embodiment, the web is entangled using hydroentanglement, which is a process used to entangle and bond fibers using hydrodynamic forces. As noted above, a binder material can be applied to the fibers of the fibrous web before laying the fibrous web, during formation of the fibrous web, and/or after the fibrous web has been bonded to form a fleece material. After forming the fleece material, heat can be applied to the fleece material in order to activate/at least partially melt the binder material to further bond the fleece material and thereby further enhance the mechanical integrity of the fleece material.

Methods for forming a nonwoven web comprising natural and synthetic fibers may include drylaid, airlaid and wetlaid methods. In some embodiments, the nonwoven fabric can be formed using a spunlaid or spunmelt process, which includes both spunbond and meltblown processes, wherein such processes are understood to typically entail melting, extruding, collecting and bonding thermoplastic polymer materials to form a fibrous nonwoven web. The technique of meltblowing is known in the art and is discussed in various patents, for example, U.S. Pat. Nos. 3,849,241 to Butin, 3,987,185 to Buntin et al., 3,972,759 to Buntin, and 4,622,259 to McAmish et al., each of which is herein incorporated by reference in its entirety. General spunbonding processes are described, for example, in U.S. Pat. Nos. 4,340,563 to Appel et al., 3,692,618 to Dorschner et al., 3,802,817 to Matsuki et al., 3,338,992 and 3,341,394 to Kinney, 3,502,763 to Hartmann, and 30 3,542,615 to Dobo et al., which are all incorporated herein by reference.

In various embodiments, the nonwoven web is made by providing a drylaid or a spunlaid web of fibers, and then needle punching the web to bond the dry laid or spun laid web. The needle punched fleece material is produced when barbed needles are pushed through the fibrous web, forcing some fibers upwards or downwards through the web by the barbed needles. The fibers punched through the web remain at their new position once the needles are withdrawn. This needling action interlocks fibers and holds the structure together by inter fiber friction forces caused by compression of the web, thereby bonding the web. By displacing a sufficient number of fibers in the web, the web is converted into a nonwoven fabric.

In certain embodiments, the nonwoven web is made by a fleece carding process with point bonding. The point bonding (e.g., using a calendar) should be limited to a relatively small portion of the surface area of the nonwoven web to maintain good porosity in the web for migration of water-soluble components through the web during oral use. In certain embodiments, the point bonding is limited to less than about 60% of the surface area of the nonwoven web (or resulting pouch), such as less than about 50%, less than about 30%, or less than about 20% (e.g., about 1% to about 50%, about 5% to about 40%, or about 10% to about 30%). An advantage of point bonding is the ability to control the porosity, flexibility and fabric strength.

In other embodiments, the nonwoven web can be subjected to hydroentangling. The term “hydroentangled” or “spunlaced” as applied to a nonwoven fabric herein defines a web subjected to impingement by a curtain of high speed, fine water jets, typically emanating from a nozzle jet strip accommodated in a pressure vessel often referred to as a manifold or an injector. This hydroentangled fabric can be characterized by reoriented, twisted, turned and entangled fibers. For example, the fibers can be hydroentangled by exposing the nonwoven web to water pressure from one or more hydroentangling manifolds at a water pressure in the range of about 10 bar to about 1000 bar. As compared to point bonding, spunlace technology, in certain embodiments, will have less impact on porosity of the web and, thus, may enhance flavor transfer through the nonwoven pouch material.

In various embodiments, the nonwoven web can be subjected to a second bonding method in order to reduce elongation of the web during processing. In certain embodiments, nonwoven webs of the present disclosure can exhibit significant elongation during high speed processing on pouching equipment. Too much elongation of the nonwoven web can cause the web to shrink during processing, such that the final product is not sized appropriately. As such, it can be necessary to modify process equipment to fit a wider roll of fleece, for example, to compensate for any shrinkage in the final product due to elongation.

In order to avoid or at least reduce such an elongation problem, in various embodiments the nonwoven web can be point bonded after the first bonding (e.g., hydroentangling) is completed. A second bonding process can increase the tensile strength of the nonwoven web and reduce elongation characteristics. In particular, a point bonding process can bond a nonwoven web by partially or completely melting the web (e.g., the heat sealable binder material) at discrete points. For example, in some embodiments, the nonwoven web can be subjected to ultrasonic bonding after initial bonding of the web. Any ultrasonic bonding system for nonwoven materials known in the art can be used to ultrasonically bond the nonwoven web. See, for example, the apparatuses and devices disclosed in U.S. Pat. Nos. 8,096,339 to Aust and 8,557,071 to Weiler, incorporated by reference herein. In some embodiments, the nonwoven web can be subjected to point bonding via embossed and/or engraved calendar rolls, which are typically heated. See, e.g., the point bonding methods incorporating the use of very high calendar pressures and embossing techniques discussed in U.S. Pat. Publ. No. 2008/0249492 to Schmidt, herein incorporated by reference in its entirety. The point bonding process is typically limited to less than about 60% of the surface area of the nonwoven web as noted above.

In certain embodiments, the processing techniques used to blend, entangle and bond the nonwoven web can also impart a desired texture to the fibrous nonwoven web material. For instance, point bonding or hydroentangling can impart a desired texture (e.g. a desired pattern) to the nonwoven web. This textured pattern can include product identifying information. In some embodiments, the product identifying information is selected from the group consisting of product brand, a company name, a corporate logo, a corporate brand, a marketing message, product strength, active ingredient, product manufacture date, product expiration date, product flavor, product release profile, weight, product code (e.g., batch code), other product differentiating markings, and combinations thereof.

Various manufacturing apparatuses and methods can be used to create a pouched product described herein. For example, US Publication No. 2012/0055493 to Novak, III et al., previously incorporated by reference in its entirety, relates to an apparatus and process for providing pouch material formed into a tube for use in the manufacture of smokeless tobacco products. Similar apparatuses that incorporate equipment for supplying a continuous supply of a pouch material (e.g., a pouch processing unit adapted to supply a pouch material to a continuous tube forming unit for forming a continuous tubular member from the pouch material) can be used to create a pouched product described herein. Representative equipment for forming such a continuous tube of pouch material is disclosed, for example, in U.S. Patent Application Publication No. US 2010/0101588 to Boldrini et al., which is incorporated herein by reference in its entirety. The apparatus further includes equipment for supplying pouched material to the continuous tubular member such that, when the continuous tubular member is subdivided and sealed into discrete pouch portions, each pouch portion includes a charge of a composition adapted for oral use. Representative equipment for supplying the filler material is disclosed, for example, in U.S. Patent Application Publication No. US 2010/0018539 to Brinkley, which is incorporated herein by reference in its entirety. In some instances, the apparatus may include a subdividing unit for subdividing the continuous tubular member into individual pouch portions and, once subdivided into the individual pouch portions, may also include a sealing unit for sealing at least one of the ends of each pouch portion. In other instances, the continuous tubular member may be sealed into individual pouch portions with a sealing unit and then, once the individual pouch portions are sealed, the continuous tubular member may be subdivided into discrete individual pouch portions by a subdividing unit subdividing the continuous tubular member between the sealed ends of serially-disposed pouch portions. Still in other instances, sealing (closing) of the individual pouch portions of the continuous tubular member may occur substantially concurrently with the subdivision thereof, using a closing and dividing unit. It is noted that in certain embodiments of the present disclosure wherein a low melting point binder material is used, the temperature required for sealing the seams of the pouched product can be less than the temperature required in conventional processes associated with conventional binder materials. As a result, the pouch manufacturing process according to the present disclosure can require less energy and/or faster production of pouched products as compared to conventional processes. For at least these reasons, certain processes of the present disclosure can be more economical than conventional processes.

An example apparatus for manufacturing an oral pouch product is illustrated in FIGS. 1-5 of U.S. Publication No. 2012/0055493 to Novak, III et al.; however, this apparatus is used in a generic and descriptive sense only and not for purposes of limitation. It should also be appreciated that the following manufacturing process and related equipment is not limited to the process order described below. In various embodiments of the present disclosure, an apparatus similar to that described in U.S. Publication No. 2012/0055493 can be configured to removably receive a first bobbin on an unwind spindle assembly, the first bobbin having a continuous length of a material, such as a pouch material, wound thereon. When the first bobbin is engaged with the apparatus, the pouch material can be routed from the first bobbin to a forming unit configured to form a continuous supply of the pouch material into a continuous tubular member defining a longitudinal axis.

As such, as the pouch material is unwound from the first bobbin, the pouch material can be directed around an arrangement of roller members, otherwise referred to herein as a dancer assembly. A forming unit can be configured to cooperate with the first bobbin and the dancer assembly to take up slack in the pouch material and to maintain a certain amount of longitudinal tension on the pouch material as the pouch material is unwound from the first bobbin and fed to the forming unit, for example, by a drive system. One of ordinary skill in the art will appreciate that, between the first bobbin and the forming unit, the pouch material can be supported, routed, and/or guided by a suitably aligned series of any number of, for example, idler rollers, guideposts, air bars, turning bars, guides, tracks, tunnels, or the like, for directing the pouch material along the desired path. Typical bobbins used by conventional automated pouch making apparatuses often contain a continuous strip of pouch material of which the length may vary. As such, the apparatus described herein can be configured so as to handle bobbins of that type and size.

The forming unit can include one or more roller members configured to direct the pouch material about a hollow shaft such that the continuous supply of the pouch material can be formed into a continuous tubular member. The forming unit can include a sealing device configured to seal, fix, or otherwise engage lateral edges of the pouch material to form a longitudinally-extending seam, thereby forming a longitudinally-extending continuous tubular member. In various embodiments, an insertion unit can be configured to introduce charges of the composition adapted for oral use into the continuous tubular member through the hollow shaft. The insertion unit may be directly or indirectly engaged with the hollow shaft.

A leading edge or end (also referred to as a laterally-extending seam) of the continuous tubular member can be closed/sealed such that a charge of composition adapted for oral use inserted by the insertion unit, is contained within the continuous tubular member proximate to the leading end. The leading end can be closed/sealed via a closing and dividing unit configured to close/seal a first portion of the continuous tubular member to form the closed leading end of a pouch member portion. The closing and dividing unit can also be configured to form a closed trailing edge or end of a previous pouch member portion. In this regard, the closing and dividing unit can also be configured to close a second portion of the continuous tubular member to form the closed trailing end of the pouch member portion. In this regard, the closing and dividing unit can close the ends, by heat-sealing, or other suitable sealing mechanism.

As illustrated in FIGS. 20-22 of U.S. Publication No. 2012/0055493 to Novak, III et al., the closing and dividing unit can be configured to divide the continuous tubular member, between the closed trailing end and the closed leading end of serially-disposed pouch member portions, along the longitudinal axis of the continuous tubular member, and into a plurality of discrete pouch member portions such that each discrete pouch member portion includes a portion of the oral composition from the insertion unit. In this regard, the closing and dividing unit can include a blade, heated wire, or other cutting arrangement for severing the continuous tubular member into discrete pouch member portions. For example, the closing and dividing unit can include first and second arm members configured to interact to close and divide the continuous tubular member.

In operation, a charge of the composition adapted for oral use (i.e., an amount suitable for an individual pouch member portion) can be supplied to the pouch member portion by an insertion unit after a leading end has been closed, but prior to the closing of a trailing end. In various embodiments, after receiving the charge of the oral composition, the discrete individual pouch member portion can be formed by closing the trailing end and severing the closed pouch member portion from the continuous tubular member such that an individual pouched product is formed.

The amount of material contained within each pouch may vary. In various embodiments, the weight of the mixture within each pouch is at least about 50 mg, for example, from about 50 mg to about 2 grams, from about 100 mg to about 1.5 grams, or from about 200 mg to about 700 mg. In certain smaller embodiments, the weight of the material within each pouch is at least about 50 mg to about 150 mg. For some larger embodiments, the weight of the material within each pouch preferably does not exceed about 300 mg to about 500 mg. In some embodiments, each pouch/container may have disposed therein a flavor agent member, as described in greater detail in U.S. Pat. No. 7,861,728 to Holton, Jr. et al., which is incorporated herein by reference. In some embodiments, an edible, orally dissolvable film as provided herein can be disposed within the cavity. Such films may be folded, crumpled, or cut/shredded (e.g., to put it in the form of a confetti-like material) in order to be readily incorporated within the pouch. See, for example, the types of materials and technologies set forth in U.S. Pat. Nos. 6,887,307 to Scott et al. and 6,923,981 to Leung et al.; and The EFSA Journal (2004) 85, 1-32; which are incorporated herein by reference. General, non-limiting methods for producing nonwoven pouches are described, for example, in U.S. Patent Application Publication No. 2016/0073689 to Sebastian et al., which is incorporated herein by reference in its entirety.

In various embodiments, the nonwoven web can be sufficiently tacky so as to create issues with high-speed pouching equipment. Therefore, in certain embodiments, a Teflon coating, or similar material, can be applied to one or more surfaces of the pouching equipment that touch the nonwoven web such as, for example, rollers, cutting instruments, and heat sealing devices in order to reduce and/or alleviate any problems associated with the pouch material sticking to the pouching equipment during processing.

In one embodiment, a continuous supply of a pouch material in the form of a nonwoven web comprising at least a portion of fibers according to the present disclosure can be provided; the pouch material is formed into a continuous tubular member by sealing the lateral edges of the pouch material such that a longitudinally-extending seam is formed. As noted herein, the seam can be formed, for example, by applying conventional heat sealing techniques to the pouch material, resulting in softening and/or melting of the heat sealable binder material in the nonwoven web to form a seal. A charge of a composition adapted for oral use can be inserted into the continuous tubular member; the continuous tubular member can be subdivided at predetermined intervals so as to form a plurality of pouch member portions, wherein each pouch member portion includes a charge of the composition. Each discrete pouch portion can then be entirely sealed such that an outer water-permeable pouch is formed that encloses the composition. This second sealing step can involve applying conventional heat sealing techniques to the pouch material, resulting in softening and/or melting of the heat sealable binder material in the nonwoven web to form a seal. Accordingly, aspects of the present disclosure are particularly configured to provide discrete pouched products. The operations described and the order of the method steps illustrated herein are not construed as limiting thereof.

The pouched products can further include product identifying information printed or dyed on the outer water-permeable pouch or imprinted (e.g., embossed, debossed, or otherwise pressed) on the outer water-permeable pouch, such as described in U.S. Pat. Appl. Pub. No. 2014/0255452 to Reddick et al., filed Mar. 11, 2013, which is incorporated by reference herein. As noted above, flavorants can also be incorporated into the nonwoven web if desired, such as by coating or printing an edible flavorant ink onto the nonwoven web. See, e.g., U.S. Pat. Appl. Pub. Nos. 2012/0085360 to Kawata et al. and 2012/0103353 to Sebastian et al., each of which is herein incorporated by reference.

The edible, orally dissolvable film can be applied to the fleece at varying stages of the pouch production process and in varying manners. In some embodiments, the film can be laminated onto a surface of the fleece before or after production of the pouch. Certain, non-limiting methods by which such lamination can be conducted include, e.g., thermal bonding and thermal/pressure application that can be combined optionally with some moisturization. In some embodiments, this lamination is conducted prior to pouch making.

In other embodiments, the edible, orally dissolvable film can be applied to the fleece by spraying, printing, or otherwise applying a liquid composition to the fleece and drying the liquid to provide a film layer thereon. The liquid composition generally comprises, e.g., film components as provided herein above (e.g., an active agent, a binder, a plasticizer, a flavorant, a taste modifier, a filler, a pH modifier, or any combination thereof), as well as a solvent. Certain sprayable/printable liquid compositions suitable for applying films in this manner can comprise, for example, a binder selected from HPMC, other cellulosics, agar, alginates, starches, natural polymer mixtures (e.g., Agaroid RS500/Ingredion, which contains agar, locust bean gum, and carrageenan), and combinations thereof. Certain sprayable/printable liquid compositions suitable for applying films in this manner can comprise one or more plasticizers and/or solvents, including, e.g., water, glycerin, propylene glycol, and the like. In some such embodiments, methods of spraying can vary and can be selected from stream, fan spray, and atomization. In some such embodiments, methods of printing can vary and can be selected from contact printing, ink jet printing, and other methods of printing.

In some embodiments, an edible, orally dissolvable film can be prepared and then simply be placed within the cavity of a fleece-based pouch before it is sealed.

Products of the present disclosure configured for oral use may be packaged and stored in any suitable packaging in much the same manner that conventional types of smokeless tobacco products are packaged and stored. For example, a plurality of packets or pouches may be contained in a cylindrical container. The storage period of the product after preparation may vary. As used herein, “storage period” refers to the period of time after the preparation of the disclosed product. In some embodiments, one or more of the characteristics of the products disclosed herein (e.g., retention of whiteness, lack of color change, retention of volatile flavor components) is exhibited over some or all of the storage period. In some embodiments, the storage period (i.e., the time period after preparation) is at least one day. In some embodiments, the storage period is from about 1 day, about 2 days, or about 3 days, to about 1 week, or from about 1 week to about 2 weeks, from about 2 weeks to about 1 month, from about 1 month to about 2 months, from about 2 months to about 3 months, from about 3 months to about 4 months, or from about 4 months to about 5 months. In some embodiments, the storage period is any number of days between about 1 and about 150. In certain embodiments, the storage period may be longer than 5 months, for example, about 6 months, about 7 months, about 8 months, about 9 months, about 10 months, about 11 months, or about 12 months.

### EXAMPLES

In the following examples, “Hydroxypropyl Methyl Cellulose A” is HPMC with a viscosity of 4.0 cPs; “Hydroxypropyl Methyl Cellulose B” is HPMC with a viscosity of 79-119 cPs; and “Hydroxypropyl Methyl Cellulose C” is HPMC with a viscosity of 80-120 cPs. It is noted that, in the examples provided herein, polyoxy(40) stearate may be substituted for glyceryl stearate and Polysorbate (80) where present in the compositions.

### Example 1: Film A

TABLE 2
Composition of Film A

| Component | Percentage by Weight |
|---|---|
| Hydroxypropyl Methyl Cellulose A | 10-30 |
| Hydroxypropyl Methyl Cellulose B | 10-30 |
| L-Theanine | 20-25 |
| Glycerin | 10-20 |
| Water | 5-20 |
| Modified Corn Starch | 0-5 |
| Polysorbate 80 | 0-5 |
| Flavor | 0-5 |
| Glycerol Monostearate | 0-5 |
| Carboxymethyl Cellulose | 2-6 |
| Sucralose | 0-5 |
| Colorant | 0-1 |

A solution of all components of Film A (as provided in Table 2) in water was prepared at about 20% solids. The solution was cast onto a flat carrier and dried to about 7% moisture to give an edible, orally dissolvable film.

Additional film comparable to Film A are also envisioned, as follows: Film A-1, with 7.5% nicotine solution instead of L-theanine (prepared from film composition with pH adjusted to about 8.5 with sodium carbonate, and resulting in a film with about 3-5 mg nicotine per ˜220 mg strip); Film A-2, with 7.5% nicotine solution instead of L-theanine (prepared from film composition with pH adjusted to about 8.5 with sodium carbonate, and resulting in a film with about 5-7 mg nicotine per ˜220 mg strip); Film A-3, with a combination of theanine and gamma-aminobutyric acid in place of the L-theanine (present in amounts of about 30-40 mg theanine/about 40-50 mg gamma-aminobutyric acid per ˜220 mg strip); Film A-4, with a combination of theanine and caffeine in place of the L-theanine (present in amounts of about 35-45 mg caffeine/about 35-45 mg L-theanine per ˜220 mg strip); and Film A-5, with a combination of caffeine and taurine in place of the L-theanine (present in amounts of about 30-40 mg caffeine/about 40-50 mg taurine per ˜220 mg strip).

### Example 2: Film-based Pouches

TABLE 3
Composition of Film B

| Component | Percentage by Weight |
|---|---|
| Hydroxypropyl Methyl Cellulose A | 30-60 |
| Hydroxypropyl Methyl Cellulose B | 15-30 |
| Glycerin | 10-20 |
| Water | 2-10 |
| Modified Corn Starch | 5-10 |
| Glycerol Monostearate | 0-5 |
| Colorant | 0-1 |

A solution of all components of Film B (as provided in Table 3) in water was prepared at about 20% solids. The solution was cast onto a flat carrier and dried to about 3% moisture to give an edible, orally dissolvable film.

Pouch A: The components of Table 4 were combined in a V-shaped mixer to give Powder A. Film B was formed into a square pouch (about 25-50 mg) and Powder Material A (about 75-125 mg), was added to the interior of the pouch. The pouch was heat sealed in a square pouch shape to enclose Powder Material A.

TABLE 4
Composition of Powder A

| Component | Percentage by Weight |
|---|---|
| Isomalt | 35-45 |
| Emdex ® (binder/filler) | 40-50 |
| Sucralose | 0-2 |
| L-Theanine | 3-5 |
| Gamma-Aminobutyric acid | 4-6 |
| Lemon Balm Extract | 3-5 |
| Sodium Chloride | 0-2 |
| Sodium Stearyl Fumarate | 0-2 |

Pouch B: The components of Table 5 were combined in a V-shaped mixer to give Powder B. Film B was formed into a square pouch (about 25-50 mg) and Powder Material B (about 75-125 mg), was added to the interior of the pouch. The pouch was heat sealed in a square pouch shape to enclose Powder Material

B.

TABLE 5
Composition of Powder B

| Component | Percentage by Weight |
|---|---|
| Isomalt | 35-45 |
| Emdex ® (binder/filler) | 40-50 |
| Sucralose | 0-2 |
| Caffeine | 1-3 |
| Encapsulated caffeine (85% caffeine) | 2-4 |
| L-Theanine | 3-6 |
| Citicoline | 0-2 |
| Panax ginseng extract | 0-2 |
| Trisodium citrate dihydrate | 2-4 |
| Sodium chloride | 0-2 |
| Sodium stearyl fumarate | 0-2 |

Pouch C: The components of Table 6 were combined in a V-shaped mixer to give Powder C. Film B was formed into a square pouch (about 25-50 mg) and Powder Material C, provided below in Table 6 (about 75-125 mg), was added to the interior of the pouch. The pouch was heat sealed in a square pouch shape to enclose Powder Material C.

TABLE 6
Composition of Powder C

| Component | Percentage by Weight |
|---|---|
| Isomalt | 35-45 |
| Emdex ® (binder/filler) | 40-50 |
| Sucralose | 0-2 |
| Caffeine | 1-3 |
| Encapsulated caffeine (85% caffeine) | 2-4 |
| Taurine | 3-6 |
| Vitamin C | 2-8 |
| Trisodium citrate dihydrate | 2-4 |
| Sodium chloride | 0-2 |
| Sodium stearyl fumarate | 0-2 |

Pouch D: The components of Table 7 were combined in an overhead mixer to give Powder Material D. Film B was formed into a square pouch (about 25-50 mg) and Powder Material D, provided below in Table 7 (about 75-125 mg), was added to the interior of the pouch. The pouch was heat sealed in a square pouch shape to enclose Powder Material D.

TABLE 7
Composition of Powder D

| Component | Percentage by Weight |
|---|---|
| Microcrystalline cellulose | 40-60 |
| Sodium chloride | 0-5 |
| Sodium alginate | 0-2 |
| Water | 2-10 |
| 7.5% aqueous nicotine solution | 5-10 |
| Sodium bicarbonate | 0-2 |
| Xylitol | 1-5 |
| Propylene glycol | 0-3 |
| Acesulfame K | 0-2 |

### Example 3: Film-modified Fleece-based Pouches

TABLE 8
Composition of Mixture to form Film C

| Component | Percentage by Weight |
|---|---|
| Water | 80-95 |
| Lumulse GMS (emulsifier/surfactant) | 0.2-2 |
| Hydroxypropyl Methyl Cellulose A | 8-10 |
| Hydroxypropyl Methyl Cellulose B | 2-5 |
| CMC | 0.2-2 |
| Glycerin (plasticizer) | 2-10 |
| Polysorbate 80 | 1-5 |
| Pure-Cote (modified starch, bulking agent, | 0.5-3 |
| film-forming agent) | |
| Sucralose (sweetener) | 0.2-2 |
| Arbocel (powdered cellulose, bulking agent) | 1-5 |
| Aqueous 7.5% nicotine solution (active agent) | 5-10* |
| Flavor | 0.1-2 |

*as 7.5% wt/total weight percentage free nicotine

This example film C was made using the components above in Table 8, by the following process: 1) heat water, 2) add surfactant and mix for 20-30 seconds, 3) add HPMC and CMC components and mix, 4) add glycerin and polysorbate when solution is lump-free, 5) add remaining ingredients and continue mixing until smooth, 6) cast film composition on mylar film and dry at about 100° C., giving a “dry film,” with a final water content of about 5-10%, 7) cut dry film to desired size (e.g., in one embodiment, about 1 inch by 0.625 inch pieces at about 100 mg weight each piece). The final product components are provided below in Table 9.

TABLE 9
Final Composition of Dried Film C

| Component | Percentage by Weight |
|---|---|
| Water | 5-10 |
| Lumulse GMS (emulsifier/surfactant) | 2-4 |
| Hydroxypropyl Methyl Cellulose A | 25-40 |
| Hydroxypropyl Methyl Cellulose B | 10-20 |
| CMC | 1-4 |
| Glycerin (plasticizer) | 15-20 |
| Lumisorb ® (polysorbate, plasticizer/emulsifier) | 5-10 |
| Pure-Cote (modified starch, bulking agent, | 3-5 |
| film-forming agent) | |
| Sucralose (sweetener) | 1-3 |
| Arbocel (powdered cellulose, bulking agent) | 5-8 |
| Nicotine (active agent) | 1-3* |
| Flavor | 1-3 |

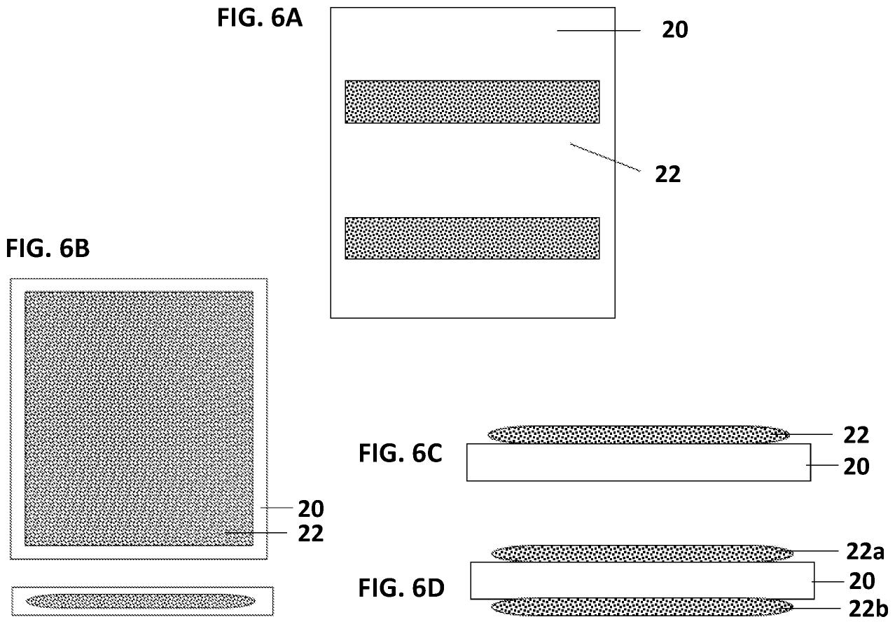

Fleeces modified with such a dried film material (C) were produced as follows, with examples of structures illustrated schematically in FIG. 6. In these figures, the fleece is depicted as component 20 and the edible, orally dissolvable film is depicted as 22 (or, in the case of FIG. 6D, 22a and 22b, where the composition and features of these two films can be the same or different). As shown, the films can be associated with the fleece in various ways, e.g., FIG. 6A, showing a laminated film on portions of a surface of the fleece (shown: strips of laminated film); FIG. 6B, showing a configuration with a film within a fleece-based pouch (i.e., on an interior surface of the fleece or positioned within the cavity of the pouch); FIG. 6C, showing a fleece piece with film positioned thereon (which film can be positioned on the inside or outside of a pouch constructed from two such fleece pieces); and FIG. 6D, showing a fleece piece with film positioned on both surfaces thereof (such that the film can be positioned on both the inside and outside of a pouch constructed from two such fleece pieces).

TABLE 10
Fleece-based pouched products produced from Film C

| Example | Structure |
|---|---|
| 3(A) | FIG. 6C (film C laminated onto fleece 1) |
| 3(B) | FIG. 6A, (strips of film C heat laminated on one |
| | side of Fleece 2), about 0.25 inch strip, about |
| | 0.75 inch center to center |
| 3(C) | FIG. 6B (pouch of Fleece 3 with internal film C, |
| | edges heat sealed, piece about 0.5 inch by 1 inch) |
| 3(D) | FIG. 6B (pouch of Fleece 2 with internal film C, |
| | edges heat sealed, piece about 0.5 inch by 1 inch) |
| 3(E) | FIG. 6B (pouch of Fleece 1 with internal film C, |
| | edges heat sealed, piece about 0.5 inch by 1 inch) |
| 3(F) | FIG. 6C (fleece latticed structure cf. FIG. 6C; |
| | may involve high gsm fleece |

Fleece 1: 34 gsm, viscose polyester blend fleece with acrylate binder
Fleece 2: 632 gsm, PBS and PLA blend fleece, with acrylate binder
Fleece 3, 34 gsm, viscose fleece with an acrylate binder

### Example 4: Film D

TABLE 11
Composition of Mixture to form Film D

| Component | Percentage by Weight |
|---|---|
| Water | 85-95 |
| Lumulse GMS (emulsifier/surfactant) | 0.5-1.5 |
| Hydroxypropyl Methyl Cellulose A | 8-10 |
| Hydroxypropyl Methyl Cellulose B | 2-5 |
| CMC | 0.5-1.5 |
| Glycerin (plasticizer) | 4-6 |
| Polysorbate 80 | 1-3 |
| Pure-Cote (modified starch, bulking agent, | 1-2 |
| film-forming agent) | |
| Sucralose (sweetener) | 0.4-1 |
| Arbocel (powdered cellulose, bulking agent) | 1-3 |
| Aqueous 12% nicotine benzoate solution | 4-6* |
| (active agent) | |
| Sodium benzoate | 4-6 |
| Flavor | 0.1-1 |

*as 12% wt/total weight percentage free nicotine

This example film D was made using the components above in Table 11, by the following process: 1) heat water, 2) add surfactant and mix for 20-30 seconds, 3) add HPMC and CMC components and mix, 4) add glycerin and polysorbate when solution is lump-free, 5) add remaining ingredients and continue mixing until smooth, 6) cast film composition on mylar film and dry at about 100° C., giving a “dry film,” with a final water content of about 5-10%, 7) cut dry film to desired size (e.g., in one embodiment, about 1 inch by 0.625 inch pieces at about 100 mg weight each piece). The final product components are provided below in Table 12.

TABLE 12
Final Composition of Dried Film D

| Component | Percentage by Weight |
|---|---|
| Water | 5-10 |
| Lumulse GMS (emulsifier/surfactant) | 2-5 |
| Hydroxypropyl Methyl Cellulose A | 25-30 |
| Hydroxypropyl Methyl Cellulose B | 8-12 |
| CMC | 1-5 |
| Glycerin (plasticizer) | 15-20 |
| Polysorbate 80 | 5-10 |
| Pure-Cote (modified starch, bulking agent, | 3-5 |
| film-forming agent) | |
| Sucralose (sweetener) | 1-4 |
| Arbocel (powdered cellulose, bulking agent) | 5-8 |
| Nicotine benzoate (active agent) | 1-3 |
| Sodium benzoate | 15-20 |
| Flavor | 0.5-3 |

### Example 5: Film E

TABLE 13
Composition of Mixture to form Film E

| Component | Percentage by Weight |
|---|---|
| Water | 60-70 |
| Lumulse GMS (emulsifier/surfactant) | 0.5-2 |
| Hydroxypropyl Methyl Cellulose A | 8-10 |
| Hydroxypropyl Methyl Cellulose B | 1-3 |
| Glycerin (plasticizer) | 5-7 |
| Polysorbate 80 | 2-4 |
| Arbocel (powdered cellulose, bulking agent) | 1-3 |
| Pure-Cote (modified starch, bulking agent, | 1-3 |
| film-forming agent) | |
| Sucralose (sweetener) | 0.5-2 |
| Aqueous nicotine solution (active agent) | 3-5* |
| Sodium benzoate | 3-5 |
| Flavor | 0.1-1 |

*as 7.5% wt/total weight percentage free nicotine

This example Film E was made using the components above in Table 13, with a total solids % of about 31.64% by the following process: 1) heat water, 2) add surfactant and mix for 20-30 seconds, 3) add HPMC(s), let solution thicken and cool (check for lumps), 4) add glycerin and polysorbate when mixture is smooth, 5) add remaining ingredients and continue mixing until smooth, 6) cast film composition on mylar film (casting thickness starting at 50 mil wet, temperature zones 100° C. top and 100° C. bottom).

Trials were conducted to achieve specification targets as follows: active content (1.5 mg nicotine), film weight (100 mg), moisture level (5-10%). Films were cast as described above, and cut into 1.25″×0.63″ strips with rounded edges, targeting 100 mg/piece weight (cut using die rolling cutter with rounded edge template). The first trial resulted in a weight of 114 mg, with moisture level 8.31%; the second trial resulted in a weight of 106 mg, with moisture level 6.92%. The final drying gave a strip with weight 0.108 g and moisture level 6.10%.

TABLE 14
Final Composition of Dried Film E

| Component | Percentage by Weight |
|---|---|
| Water | 4-6 |
| Lumulse GMS (emulsifier/surfactant) | 2-4 |
| Hydroxypropyl Methyl Cellulose A | 25-30 |
| Hydroxypropyl Methyl Cellulose B | 5-8 |
| Glycerin (plasticizer) | 18-20 |
| Polysorbate 80 | 6-8 |
| Arbocel (powdered cellulose, bulking agent) | 3-5 |
| Pure-Cote (modified starch, bulking agent, | 3-5 |
| film-forming agent) | |
| Sucralose (sweetener) | 6-8 |
| Nicotine (active agent) | 1-3 |
| Sodium benzoate | 10-15 |
| Flavor | 1-3 |

### Example 6: Film F

TABLE 15
Composition of Mixture to form Film F

| Component | Percentage by Weight |
|---|---|
| Water | 70-80 |
| Lumulse GMS (emulsifier/surfactant) | 0.5-2 |
| Hydroxypropyl Methyl Cellulose A | 8-10 |
| Hydroxypropyl Methyl Cellulose B | 1-3 |
| Glycerin (plasticizer) | 5-7 |
| Polysorbate 80 | 2-4 |
| Vivapur (powdered cellulose, bulking agent) | 1-3 |
| Pure-Cote (modified starch, bulking agent, | 1-3 |
| film-forming agent) | |
| Sucralose (sweetener) | 0.1-1 |
| Xylitol | 1-3 |
| Sodium chloride | 0.1-0.5 |
| Nicotine benzoate solution (active agent) | 3-5* |
| Sodium benzoate | 3-5 |
| Flavor | 0.5-1 |
| Colorant | 0.01-0.1 |

*as 12% wt/total weight percentage free nicotine

This example Film F was made using the components above in Table 15, with a total solids % of about 35.53% by the following process: 1) heat water, 2) add surfactant and mix for 20-30 seconds, 3) add HPMC(s), let solution thicken and cool (check for lumps), 4) add glycerin and polysorbate when mixture is smooth, 5) add remaining ingredients and continue mixing until smooth, 6) cast film composition on mylar film (casting thickness starting at 50 mil wet, temperature zones 100° C. top and 100° C. bottom).

This example was designed to achieve specification targets as follows: active content (2.0 mg nicotine), film weight (140 mg). Films were cast as described above, and cut into 1.25″×0.43″ strips with rounded edges, targeting 140 mg/piece weight (cut using die rolling cutter with rounded edge template).

TABLE 16
Final Composition of Dried Film F

| Component | Percentage by Weight |
|---|---|
| Water | 4-6 |
| Lumulse GMS (emulsifier/surfactant) | 2-4 |
| Hydroxypropyl Methyl Cellulose A | 25-30 |
| Hydroxypropyl Methyl Cellulose B | 5-8 |
| Glycerin (plasticizer) | 18-20 |
| Polysorbate 80 | 6-8 |
| Vivapur (powdered cellulose, bulking agent) | 3-5 |
| Pure-Cote (modified starch, bulking agent, | 3-5 |
| film-forming agent) | |
| Sucralose (sweetener) | 6-8 |
| Nicotine (active agent) | 1-3 |
| Sodium chloride | 0.1-0.5 |
| Sodium benzoate | 10-15 |
| Flavor | 1-3 |
| Colorant | 0.01-0.1 |

### Example 7: Film G

TABLE 17
Composition of Mixture to form Film G

| Component | Percentage by Weight |
|---|---|
| Water | 70-80 |
| Lumulse GMS (emulsifier/surfactant) | 0.5-2 |
| Hydroxypropyl Methyl Cellulose A | 8-10 |
| Hydroxypropyl Methyl Cellulose C | 1-3 |
| Glycerin (plasticizer) | 5-7 |
| Polysorbate 80 | 2-4 |
| Vivapur (powdered cellulose, bulking agent) | 1-3 |
| Pure-Cote (modified starch, bulking agent, | 1-3 |
| film-forming agent) | |
| Sucralose (sweetener) | 0.1-1.0 |
| Xylitol | 2-4 |
| Sodium chloride | 0.1-0.5 |
| Nicotine monomenthyl succinate solution | 2-8* |
| (active agent) | |
| Flavor | 0.1-1 |
| Color | 0.01-0.1 |

*as 10.1% wt/total weight percentage free nicotine with 15.1% monomenthyl succinate, 74% water

This example Film G was made using the components above in Table 17, with a total solids % of about 32.04% by the following process: 1) heat water, 2) add surfactant and mix for 20-30 seconds, 3) add HPMC(s), let solution thicken and cool (check for lumps), 4) add glycerin and polysorbate when mixture is smooth, 5) add remaining ingredients and continue mixing until smooth, 6) cast film composition on mylar film (casting thickness starting at 50 mil wet, temperature zones 100° C. top and 100° C. bottom).

This example was designed to achieve specification targets as follows: active content (2.0 mg nicotine), film weight (140 mg). Films were cast as described above, and cut into 1.25″×0.43″ strips with rounded edges, targeting 140 mg/piece weight (cut using die rolling cutter with rounded edge template).

TABLE 18
Final Composition of Dried Film G

| Component | Percentage by Weight |
|---|---|
| Water | 4-6 |
| Lumulse GMS (emulsifier/surfactant) | 2-4 |
| Hydroxypropyl Methyl Cellulose A | 30-35 |
| Hydroxypropyl Methyl Cellulose B | 5-8 |
| Glycerin (plasticizer) | 20-22 |
| Polysorbate 80 | 7-9 |
| Vivapur (powdered cellulose, bulking agent) | 3-5 |
| Pure-Cote (modified starch, bulking agent, | 3-5 |
| film-forming agent) | |
| Sucralose (sweetener) | 1-2 |
| Xylitol | 8-10 |
| Sodium Chloride | 0.1-1.0 |
| Nicotine monomenthyl succinate (active agent) | 1-3 |
| Flavor | 1-3 |
| Color | 0.01-0.1 |

### Example 8: Film H

TABLE 19
Composition of Mixture to form Film H

| Component | Percentage by Weight |
|---|---|
| Water | 70-80 |
| Lumulse GMS (emulsifier/surfactant) | 0.5-2 |
| Hydroxypropyl Methyl Cellulose A | 8-10 |
| Hydroxypropyl Methyl Cellulose B | 1-3 |
| Glycerin (plasticizer) | 5-7 |
| Polysorbate 80 | 2-4 |
| Vivapur (powdered cellulose, bulking agent) | 1-3 |
| Pure-Cote (modified starch, bulking agent, | 1-3 |
| film-forming agent) | |
| Sucralose (sweetener) | 0.1-1.0 |
| Xylitol | 2-4 |
| Sodium chloride | 0.1-0.5 |
| Nicotine monomenthyl succinate solution | 5-7* |
| (active agent) | |
| Flavor | 0.1-1 |
| Color | 0.01-0.1 |

*as 6.7% wt/total weight percentage free nicotine in 10.6% monomenthyl succinate/83% water

This example Film H was made using the components above in Table 19, with a total solids % of about 31.57% by the following process: 1) heat water, 2) add surfactant and mix for 20-30 seconds, 3) add HPMC(s), let solution thicken and cool (check for lumps), 4) add glycerin and polysorbate when mixture is smooth, 5) add remaining ingredients and continue mixing until smooth, 6) cast film composition on mylar film (casting thickness starting at 50 mil wet, temperature zones 100 ° C. top and 100° C. bottom).

This example was designed to achieve specification targets as follows: active content (2.0 mg nicotine), film weight (140 mg). Films were cast as described above, and cut into 1.25″×0.43″ strips with rounded edges, targeting 140 mg/piece weight (cut using die rolling cutter with rounded edge template).

TABLE 20
Final Composition of Dried Film H

| Component | Percentage by Weight |
|---|---|
| Water | 4-6 |
| Lumulse GMS (emulsifier) | 2-4 |
| Hydroxypropyl Methyl Cellulose A | 30-35 |
| Hydroxypropyl Methyl Cellulose B | 5-8 |
| Glycerin (plasticizer) | 20-22 |
| Polysorbate 80 | 7-9 |
| Vivapur (powdered cellulose, bulking agent) | 3-5 |
| Pure-Cote (modified starch, bulking agent, | 3-5 |
| film-forming agent) | |
| Sucralose (sweetener) | 1-2 |
| Xylitol | 8-10 |
| Sodium Chloride | 0.1-1.0 |
| Nicotine monomenthyl succinate (active agent) | 1-3 |
| Flavor | 1-3 |
| Color | 0.01-0.1 |

### Example 9: Film-Modified Fleece-Based Pouch (Active Agent Inside Pouch)

A pouch was prepared using a fleece and powder fill material, and the pouch was then treated with a film-forming solution and dried to give a film-modified fleece-based pouch. The fleece was prepared, and pouches were prepared therefrom including about 350 mg of powder fill material (Powder E, the composition of which is provided in Table 21, below).

TABLE 21
Composition of Powder E

| Component | Percentage by Weight |
|---|---|
| Cellulose, 700 μm | 70-80 |
| Sodium chloride | 0-5 |
| Aqueous 12% nicotine solution | 10-20* |
| Propylene glycol | 0-3 |
| Sodium bicarbonate | 0-2 |
| Xylitol | 0-3 |
| Sucralose | 0-2 |

*as 12% wt/total weight percentage free nicotine solution

Separately, a print solution was prepared (Print Solution A, the compositions of which is provided in Table 22 below). Print Solution A was prepared by dispersing starch and Agaroid RS500 in cold water. Polyoxy(40) stearate, salt, and sucralose were added to the mixture, and the mixture was heated to ˜193° F. with stirring for about 5 minutes. The mixture was cooled to ˜115° F. and flavor was added with stirring;

the mixture was then filtered through a 600 mm stainless steel mesh.

TABLE 22
Composition of Print Solution A

| Component | Percentage by Weight |
|---|---|
| Water | 85-95 |
| Flavor | 2-8 |
| Polyoxy(40) stearate | 0.5-3 |
| Starch | 2-6 |
| Agaroid RS500 | 0.1-1 |
| Sodium chloride | 0-3 |
| Sucralose | 0-2 |

Individual pouches were printed with Print Solution A using a BioDot AD1500 printer using about 2 psi pressure with valve and syringe temperatures set to about 70° C. A rectangular shape was printed on one side of the pouch (e.g., as shown in FIG. 5F), giving about 120 mg wet add-on of Print Solution A per pouch, which was then dried to give a film-modified fleece-based pouch.

In a further example, Powder E was used to make an oral pouched produced (comprising about 342 g of Powder E and about 30 mg of fleece material). Print Solution A was sprayed on the outside surface of the pouch to give a film-modified fleece-based pouch, which had a final weight of about 420 mg (including pouch fill, fleece, and coating).

### Example 10: Film-Modified Fleece-Based Pouch (Active Agent on Fleece)

A pouch was prepared using a fleece and powder fill material, and the pouch was then treated with a film-forming solution and dried to give a film-modified fleece-based pouch. The fleece was prepared, and pouches were prepared therefrom including about 350 mg of non-nicotine powder fill material.

Separately, a print solution was prepared (Print Solution B, the composition of which is provided in Table 23 below). Print Solution B was prepared by dispersing sodium alginate in cold water using a blender; the solution was deaerated by standing overnight. Nicotine tocopherol succinate was made by mixing at about 75° C. about 260 g nicotine in propylene glycol, about 210 g tocopherol succinate, and water, giving a homogeneous liquid. The other components of Print Solution B were mixed in by hand stirring.

TABLE 23
Composition of Print Solution A

| Component | Percentage by Weight |
|---|---|
| Sodium alginate | 1-3 |
| Glycerin | 1-5 |
| Water | 70-80 |
| Microcrystalline Cellulose | 2-6 |
| Color | 0-1 |
| Aqueous nicotine tocopherol succinate solution | 15-20* |

*as 12% wt/total weight percentage free nicotine solution

Pouches were printed with Print Solution B using about 20 psi pressure with valve and syringe temperatures set to about 60° C. A circular shape was printed on one side of the pouch (e.g., as shown in FIG. 5E), giving about 100 mg wet add-on of Print Solution B per pouch, which was then dried to give a film-modified fleece-based pouch containing nicotine tocopherol succinate with about 2 mg nicotine.

### Example 11: Film I

TABLE 24
Composition of Film I

| Component | Percentage by Weight |
|---|---|
| Hydroxypropyl Methyl Cellulose A | 10-30 |
| Hydroxypropyl Methyl Cellulose C | 2-8 |
| Agaroid 300 | 2-15 |
| Glycerin | 15-20 |
| Water | 2-8 |
| Glyceryl Stearate | 0-5 |
| Polysorbate 80 | 5-10 |
| MCC | 2-8 |
| Sodium chloride | 1-3 |
| Starch | 2-8 |
| Xylitol | 5-10 |
| Sucralose | 0-3 |
| Nicotine benzoate | 1-3 |
| Sodium benzoate | 5-15 |
| Color | 0-1 |
| Flavor | 0-3 |

Water was heated and glyceryl stearate was added; the mixture was stirred for about 20-30 seconds. HPMC-A and HPMC-C were added and with continued stirring, the mixture began to thicken and cool. When the mixture was free of lumps/smooth, glycerin and polysorbate 80 were added; remaining ingredients were then added and stirring was continued until the mixture was smooth. The mixture was cast onto a flat glass or mylar carrier and dried; the dried material was cut into individual edible, orally dissolvable film strips of about 1.25″×0.43″ and about 140 mg. The presence of Agaroid in this sample beneficially reduced strip oral dissolution time and gave a clean finish.

Many modifications and other embodiments of the invention will come to mind to one skilled in the art to which this invention pertains having the benefit of the teachings presented in the foregoing description. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

1. An oral product comprising an edible film,
wherein the edible film comprises:
a binder in an amount of at least about 30 percent by weight;
a plasticizer in an amount of at least about 5 percent by weight; and
an active agent;
wherein the edible film is orally dissolvable.

2. The oral product of claim 1, wherein the binder is selected from the group consisting of film-forming polysaccharides, starch, modified starch, methyl cellulose, modified cellulose, pullulan, pectin, carrageenan, alginate, gums, agar, and combinations thereof.

3. The oral product of claim 1, wherein the binder comprises hydroxypropylmethylcellulose, carboxymethylcellulose, modified corn starch, or any combination thereof.

4. The oral product of claim 1, wherein the binder is present in an amount of at least about 35 percent by weight.

5. The oral product of claim 1, wherein the plasticizer is selected from the group consisting of glycerin, propylene glycol, and combinations thereof.

6. The oral product of claim 1, wherein the edible film further comprises a filler selected from the group consisting of carbohydrates, cellulose powder, fiber, starch, maltodextrin, polyglycitols, polysaccharides, minerals, and combinations thereof.

7. The oral product of claim 1, wherein the edible film further comprises a processing aid selected from the group consisting of surfactants and emulsifiers.

8. The oral product of claim 7, wherein the processing aid is selected from lecithin, polysorbate, polyoxyl stearate, glycerol monostearate, and combinations thereof.

9. The oral product of claim 1, wherein the edible film further comprises a flavorant, a colorant, a sweetener, or any combination thereof.

10. The oral product of claim 1, wherein the active agent is selected from the group consisting of a nicotine component, botanicals, stimulants, nutraceuticals, amino acids, vitamins, cannabinoids, cannabimimetics, terpenes, and combinations thereof.

11. The oral product of claim 10, wherein the active agent is a nicotine component selected from nicotine benzoate and nicotine polacrilex.

12. The oral product of claim 1, comprising a basic amine and an organic acid, an alkali metal salt of an organic acid, or a combination thereof, wherein the organic acid has a logP value of from about 1.0 to about 12.0 and at least a portion of the basic amine is associated with at least a portion of the organic acid or the alkali metal salt thereof, the association in the form of a basic amine-organic acid salt, an ion pair between the basic amine and a conjugate base of the organic acid, or both.

13. The oral product of claim 12, wherein the organic acid has a logP value of from about 1.4 to about 4.5.

14. The oral product of claim 12, wherein the organic acid has a logP value of from about 4.5 to about 8.0, and wherein the composition further comprises a solubility enhancer.

15. The oral product of claim 12, wherein the organic acid is an alkyl carboxylic acid, an aryl carboxylic acid, an alkyl sulfonic acid, an aryl sulfonic acid, a menthyl or tocopherol monoester of a dicarboxylic acid, or a combination of any thereof.

16. The oral product of claim 12, wherein the organic acid is octanoic acid, decanoic acid, benzoic acid, heptanesulfonic acid, tocopherol succinate, monomenthyl succinate, monomenthyl fumarate, monomenthyl glutarate, or a combination of any thereof.

17. The oral product of claim 12, wherein the basic amine is nicotine.

18. The oral product of claim 10, wherein the active agent comprises a stimulant.

19. The oral product of claim 18, wherein the stimulant is selected from the group consisting of caffeine, theanine, or a combination thereof.

20. The oral product of claim 18 wherein the active agent comprises one or more of theanine, gamma-aminobutyric acid, caffeine, and taurine.

21. The oral product of claim 1, consisting essentially of the edible film.

22. The oral product of claim 1, in the form of a pouched product comprising the edible film, the oral product comprising an outer pouch defining a cavity,
wherein the outer pouch comprises a fleece material.

23. The oral product of claim 22, wherein the edible film is a coating on at least a portion of an interior or exterior surface of the fleece material.

24. The oral product of claim 22, wherein the edible film is a coating on substantially all of the interior or exterior surface of the fleece material.

25. The oral product of claim 22, wherein the edible film is a coating on specific areas of the interior or exterior surface of the fleece material.

26. The oral product of claim 22, wherein the edible film is within the cavity of the pouch.
